(19) United States
(12) Patent Application Publication
(10) Pub. No.: US 2024/0217985 A1
(43) Pub. Date: Jul. 4, 2024

(54) CRYSTALLINE FORM OF AN MDM2-p53 INHIBITOR AND PHARMACEUTICAL COMPOSITIONS

(71) Applicant: Boehringer Ingelheim International GmbH, Ingelheim am Rhein (DE)

(72) Inventors: Fredrik Lars NORDSTROM, Ridgefield, CT (US); Norbert BIRK, Rot an der Rot (DE); Manuel HENRY, Schemmerhofen (DE); Brian J. LINEHAN, Cheshire, CT (US); Peter SCHNEIDER, Ulm-Einsingen (DE)

(21) Appl. No.: 18/391,748

(22) Filed: Dec. 21, 2023

Related U.S. Application Data
(60) Provisional application No. 63/476,661, filed on Dec. 22, 2022.

(30) Foreign Application Priority Data
Mar. 13, 2023 (EP) 23161577.4

(51) Int. Cl. C07D 487/22

(52) U.S. Cl. CPC C07D 487/22

(57) ABSTRACT

The present invention relates to a crystalline form of an MDM2-p53 inhibitor and methods for its preparation. Furthermore, the invention relates to pharmaceutical compositions comprising an MDM2-p53 inhibitor, preferably the crystalline form of the present invention, and at least one pharmaceutically acceptable excipient as well as to methods for their preparation. The crystalline form of the MDM2-p53 inhibitor of the present invention and the pharmaceutical compositions of the present invention can be used as a medicament, in particular for the treatment of cancer in patients bearing tumors with TP53 wild-type status.

### RELATED APPLICATION DISCLOSURE

This application claims the benefit of U.S. Provisional Application Ser. No. 63/476,661, filed Dec. 22, 2022, and European Patent Application No. EP23161577.4, filed Mar. 13, 2023, each of which is hereby incorporated by reference in its entirety.

### FIELD OF THE INVENTION

The present invention relates to a crystalline form of an MDM2-p53 inhibitor and methods for its preparation. Furthermore, the invention relates to pharmaceutical compositions comprising an MDM2-p53 inhibitor, preferably the crystalline form of the present invention, and at least one pharmaceutically acceptable excipient as well as to methods for their preparation. The crystalline form of the MDM2-p53 inhibitor of the present invention and the pharmaceutical compositions of the present invention can be used as a medicament, in particular for the treatment of cancer in patients bearing tumors with TP53 wild-type status.

### BACKGROUND OF THE INVENTION

(2′S,3′S,3a′S,10a′S)-6-chloro-3′-(3-chloro-2-fluorophenyl)-1′-(cyclopropylmethyl)-6′-methyl-2-oxo-1,2,3′,3a′,10′,10a′-hexahydro-1′H-spiro[indole-3,2′-pyrrolo[2′,3′:4,5]pyrrolo[1,2-b]indazole]-7′-carboxylic acid (IUPAC name) is an MDM2-p53 inhibitor intended for the treatment of cancer in patients bearing tumors with TP53 wild-type status. This MDM2-p53 inhibitor is now also known under its INN brigimadlin. It can be represented by the following chemical structure according to Formula (I) (=Compound A)

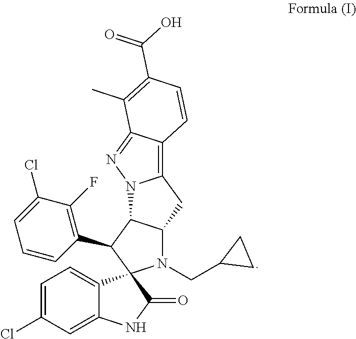 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

WO 2017/060431 A1 discloses Compound A and its synthesis, wherein the last step comprises ester saponification yielding Compound A after reversed phase HPLC (WO 2017/060431 A1, page 147, Compound Ja-34).

Different solid-state forms of an active pharmaceutical ingredient often possess different properties. Differences in physicochemical properties of solid-state forms can play a crucial role for the improvement of pharmaceutical compositions, for example, pharmaceutical formulations with improved dissolution profile and bioavailability or with improved stability or shelf-life can become accessible due to an improved solid-state form of an active pharmaceutical ingredient. Also processing or handling of the active pharmaceutical ingredient during the formulation process may be improved. New solid-state forms of an active pharmaceutical ingredient can thus have desirable processing properties. They can be easier to handle, better suited for storage, and/or allow for better purification, compared to previously known solid-state forms.

The thermodynamically most stable polymorph of an active pharmaceutical ingredient is often used for the preparation of a drug product, since phase transitions during pharmaceutical standard processes like sieving, milling, granulation, blending and compaction or during storage can be reduced to the extent possible. However, a huge drawback connected with the thermodynamically most stable form of a drug substance is the fact, that it is the least soluble form, which usually translates into decreased bioavailability. This is especially critical for low solubility compounds, such as Compound A, which shows low aqueous solubility across the physiological pH range and therefore belongs to class II drugs (low solubility, high permeability) of the biopharmaceutical classification system.

It is thus one objective of the present invention to provide a solid-state form of Compound A, which possesses improved physicochemical properties. A particular objective of the present invention is to provide a solid-state form of Compound A which is chemically and physically stable, e.g. against moisture and temperature stress, possesses an acceptable degree of hygroscopicity, and at the same time is sufficiently bioavailable when administered orally.

Furthermore, Compound A is lipophilic at physiological pH which renders the manufacture of an oral solid dosage form comprising Compound A challenging.

Hence, another objective of the present invention is to provide an oral solid dosage form comprising Compound A, in particular a suitable solid-state form of Compound A, in which the active pharmaceutical ingredient is released and dissolved quickly in order to ensure sufficient oral bioavailability.

### BRIEF SUMMARY OF THE INVENTION

The present invention solves one or more of the above-mentioned problems by providing a crystalline form of Compound A. The crystalline materials of the present invention were found to be physically and chemically stable against temperature and humidity stress. E.g. the crystalline form of Compound A obtained according to the procedure of Example 1 was stable when subjected to accelerated stress conditions of 40° C./75% relative humidity for 6 months (see Example 8.1, Table 7 and FIG. 6 hereinafter), 25° C./60% relative humidity for 6 months (see Example 8.2) and 60° C./80% relative humidity for 3 days (see Example 8.3) In addition, it was physically and chemically stable when exposed to temperature stress conditions of 70° C. for 3 weeks and 105° C. for 24 hours (see Example 8.4). Also, the crystalline form of Compound A obtained according to the procedure of Example 3 was physically and chemically stable when exposed to stress conditions of 60° C./80% relative humidity for 3 days (see Example 8.3) and 105° C. for 24 hours (see Example 8.4), respectively.

Furthermore, the crystalline form of the present invention possesses an acceptable degree of hygroscopicity even when exposed to high relative humidity. For example, it takes up only about 3.1 to 3.6% (w/w) of water when subjected to a relative humidity as high as 90% (see Example 9, FIG. 14 and FIG. 15 hereinafter). In addition, despite being a thermodynamically sink modification, it was surprisingly found that the crystalline form of the present invention exhibits a bioaccessibility profile comparable to the one of amorphous Compound A (see Comparative Example 1, FIG. 16 and FIG. 17 hereinafter). Since the oral bioavailability of a BCS class II compound like Compound A is mainly limited by its low solubility and not by its permeability the crystalline form of Compound A of the present invention and amorphous Compound A, both having comparable bioaccessibility profiles under biorelevant conditions, will also exhibit comparable bioavailability after oral administration.

Hence, the crystalline form of Compound A of the present invention is not only the most stable solid-state form of Compound A but also combines the advantageous properties of having high chemical and physical stability against moisture and temperature stress, and possessing an acceptable degree of hygroscopicity even at high relative humidity, with good oral bioavailability and is therefore particularly suited for the preparation of a safe and efficacious oral solid dosage form comprising Compound A.

The crystalline form of the present invention also possesses one or more additional improved properties selected from the group consisting of melting point, residual solvent content, hygroscopicity, solubility, dissolution, morphology, crystallinity, flowability, bulk density, compactibility and wettability. A particular advantageous property of the crystalline form of the present invention concerns its low residual solvent content, which is in accordance with regulatory guidelines for residual solvents (e.g. ICH guideline Q3C(R6) on impurities: guideline for residual solvents). Finally, the crystalline form of Compound A of the present invention can be prepared in reliable quality, good yield (see e.g. Example 1) and is therefore economically manufacturable also on industrial scale.

The present invention also provides oral solid dosage forms, in particular granules and tablets such as film-coated tablets, containing Compound A, in particular the crystalline form of Compound A of the present invention, which ensure sufficient release and dissolution of the active pharmaceutical ingredient. In this regard, the inventors of the present invention found that a certain amount of an anionic surfactant such as sodium lauryl sulfate in combination with other excipients contribute to ensure good dissolution rates of the drug substance (see Comparative Examples 2 and 3 as well as FIGS. 18 and 19, respectively).

In addition, the oral solid dosage forms of the present invention containing Compound A have no special requirements for production equipment, have a simple preparation process, a stable product and low production costs.

### Abbreviations

XRPD X-ray powder diffractogram or X-ray powder diffraction
FTIR Fourier transform infrared spectroscopy
DSC differential scanning calorimetry
TGA thermogravimetric analysis
GMS gravimetric moisture sorption
ssNMR solid-state nuclear magnetic resonance
RH relative humidity
RT room temperature
PSD particle size distribution
IPA isopropyl alcohol=isopropanol=2-propanol
EtOH ethanol
MeOH methanol
n-PrOH n-propanol=1-propanol
ACT acetone
ACN acetonitrile
cps counts per second
rpm rotations per minute
ppm parts per million
MCC microcrystalline cellulose
FCTs film-coated tablets
FaSSGF fasted state simulated gastric fluid
FaSSIF fasted state simulated intestinal fluid
BCS biopharmaceutical classification system
SLS sodium lauryl sulfate
(w/w) weight by weight
wt % weight percent
DIPE diisopropyl ether

### Definitions

In the context of the present invention the following definitions have the indicated meaning, unless explicitly stated otherwise:

The term “Compound A” as used herein refers to (2′S,3′S,3a′S,10a′S)-6-chloro-3′-(3-chloro-2-fluorophenyl)-1′-(cyclopropylmethyl)-6′-methyl-2-oxo-1,2,3′,3a′,10′,10a′-hexahydro-1′H-spiro[indole-3,2′-pyrrolo[2′,3′:4,5]pyrrolo[1,2-b]indazole]-7′carboxylic acid (also spiro[3H-indole-3,2′(1′H)-pyrrolo[2′,3′:4,5]pyrrolo[1,2-b]indazole]-7′-carboxylic acid, 6-chloro-3′-(3-chloro-2-fluorophenyl)-1′-(cyclopropylmethyl)-1,2,3′,3a′,10′,10a′-hexahydro-6′-methyl-2-oxo, (2′S,3′S, 3a′S,10a′S)) according to Formula (I) disclosed herein. In case Compound A of the present invention is depicted in the form of a chemical name and as a formula, in case of any discrepancy the formula shall prevail. Compound A is now also known under its INN brigimadlin. A process for the preparation of Compound A is disclosed in WO 2017/060431 A1.

The term “free compound” refers to a compound in neutral, non-salt form. For example, the term “free Compound A” as used herein, refers to Compound A in neutral, non-salt form.

“A pharmaceutically acceptable salt” of Compound A as used herein refers to those salts which are appropriate for use in a pharmaceutical composition and that are compatible with the pharmaceutical composition of the present invention. Such salts may be obtained, for example, by reaction of Compound A with a suitable base in a suitable solvent. For example such salts include salts from ammonia, L-arginine, betaine, benethamine, benzathine, calcium hydroxide, choline, deanol, diethanolamine, diethylamine, 2-(diethylamino)ethanol, 2-(dimethylamino)ethanol, 2-aminoethanol, ethylenediamine, N-ethylglucamine, hydrabamine, 1H-imidazole, lysine (L-lysine), proline (L-proline), magnesium hydroxide, 4-2-(hydroxyethyl)morpholine, morpholine, piperazine, potassium hydroxide, 1-(2-hydroxyethyl)pyrrolidine, 1-(2-hydroxyethyl)pyrrolidone, sodium hydroxide, triethanolamine, tromethamine, zinc hydroxide, and the like.

As used herein, the term “measured at a temperature in the range of from 20 to 30° C.” refers to a measurement under standard conditions. Typically, standard conditions mean a temperature in the range of from 20 to 30° C., i.e., at room temperature. Standard conditions can mean a temperature of about 22° C. Standard conditions can also mean a temperature of about 25° C. Typically, standard conditions can additionally mean a measurement under about 30-70% relative humidity, preferably about 30-50% relative humidity and most preferably about 40% relative humidity.

As used herein the term “room temperature” refers to a temperature in the range of from 20 to 30° C.

The term “reflection” with regard to powder X-ray diffraction as used herein, means peaks in an X-ray diffractogram, which are caused at certain diffraction angles (Bragg angles) by constructive interference from X-rays scattered by parallel planes of atoms in solid material, which are distributed in an ordered and repetitive pattern in a long-range positional order. Such a solid material is classified as crystalline material, whereas amorphous material is defined as solid material, which lacks long-range order and only displays short-range order, thus resulting in broad scattering. According to literature, long-range order e.g. extends over approximately 100 to 1000 atoms, whereas short-range order is over a few atoms only (see “Fundamentals of Powder Diffraction and Structural Characterization of Materials” by Vitalij K. Pecharsky and Peter Y. Zavalij, Kluwer Academic Publishers, 2003, page 3).

The term “essentially the same” with reference to X-ray powder diffraction means that variabilities in reflection positions and relative intensities of the reflections are to be taken into account. For example, a typical precision of the 2-theta values is in the range of ±0.2° 2-theta, preferably in the range of ±0.1° 2-theta. Thus, a reflection that usually appears at 9.3° 2-theta for example can appear between 9.1 and 9.5° 2-theta, preferably between 9.2 and 9.4° 2-theta on most X-ray diffractometers under standard conditions. Furthermore, one skilled in the art will appreciate that relative reflection intensities will show inter-apparatus variability as well as variability due to degree of crystallinity, preferred orientation, sample preparation and other factors known to those skilled in the art and should be taken as qualitative measure only.

The term “solid-state form” as used herein refers to any crystalline and/or amorphous phase of a compound.

As used herein, the term “amorphous” refers to a solid-state form of a compound that is not crystalline. An amorphous compound possesses no long-range order and does not display a definitive X-ray diffraction pattern with reflections.

A solid-state form of Compound A may be referred to herein as being characterized by graphical data “as shown in” a figure. Such data include, for example, XRPDs, ssNMR spectra, FTIR spectra, Raman spectra, DSCs, TGAs and GMS isotherms. The person skilled in the art understands that factors such as variations in instrument type, response and variations in sample directionality, sample concentration, sample purity, sample history and sample preparation may lead to variations for such data when presented in graphical form, for example variations relating to the exact peak positions and intensities. However, a comparison of the graphical data in the figures herein with the graphical data generated for an unknown solid-state form and the confirmation that two sets of graphical data relate to the same crystal form is well within the knowledge of a person skilled in the art.

As used herein, the term “mother liquor” refers to the solution remaining after crystallization of a solid.

A “predetermined amount” as used herein with regard to Compound A, or a pharmaceutically acceptable salt thereof, or a crystalline form thereof, refers to the initial amount of Compound A, a pharmaceutically acceptable salt thereof, or a crystalline form thereof, used for the preparation of a pharmaceutical composition having a desired dosage strength of Compound A.

The term “effective amount” as used herein with regard to Compound A, a pharmaceutically acceptable salt thereof, or a crystalline form thereof, encompasses an amount of Compound A, or a pharmaceutically acceptable salt thereof, or a crystalline form thereof, which causes the desired therapeutic and/or prophylactic effect.

The term “particle size distribution” as used herein refers to a list of values or a mathematical function that defines the relative amount, typically in mass or volume, of particles present in a sample according to size. Particle size distribution can be characterized by one or more values, such as D90, D50 or D10. The particle size distribution may be determined by means well known to the skilled artisan e.g. by laser diffraction.

“D90”, as used herein, describes the value of particle size at which 90% of the total volume of particles is comprised of particles no larger than the indicated size.

“D50”, as used herein, describes the value of particle size at which 50% of the total volume of particles is comprised of particles no larger than the indicated size.

“D10”, as used herein, describes the value of particle size at which 10% of the total volume of particles is comprised of particles no larger than the indicated size.

The term “oral solid dosage form” as used herein refers to a solid formulation suitable for oral administration to a human.

The term “pharmaceutically acceptable excipient” as used herein refers to substances, which do not show a significant pharmacological activity at the given dose and that are added to a pharmaceutical composition in addition to the active pharmaceutical ingredient. Excipients may take the function of vehicle, diluent, release agent, disintegrating agent, dissolution modifying agent, absorption enhancer, stabilizer or a manufacturing aid among others. Excipients may include fillers (diluents), binders, disintegrants, lubricants and surfactants.

The terms “filler” or “diluent” as used herein refer to substances that are used to dilute the active pharmaceutical ingredient prior to delivery. Diluents and fillers can also serve as stabilizers.

As used herein the term “binder” refers to substances which bind the active pharmaceutical ingredient and pharmaceutically acceptable excipient together to maintain cohesive and discrete portions.

The terms “disintegrant” or “disintegrating agent” as used herein refers to substances which, upon addition to a solid pharmaceutical composition, facilitate its break-up or disintegration after administration and permits the release of the active pharmaceutical ingredient as efficiently as possible to allow for its rapid dissolution.

The term “lubricant” as used herein refers to substances which are added to a powder blend to prevent the compacted powder mass from sticking to the equipment during tableting or encapsulation process. They aid the ejection of the tablet from the dies and can improve powder flow.

The term “surfactant” as used herein refers to substances which reduce the surface tension or interfacial tension.

The term “hypromellose” as used herein refers to hydroxypropyl methylcellulose.

As used herein, the term “about” means within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range.

The terms “subject” or “patient” which may be used interchangeably herein refer to a human, e.g. a human suffering from, at risk of suffering from, or potentially capable of suffering from cancer.

The term “treating” or “treatment” as used herein denotes to arrest, delay the onset (i.e. the period prior to clinical manifestation of a disease) and/or reduce the risk of developing or worsening a disease, or comprises relieving, reducing or alleviating at least one symptom in a subject or effecting a delay of progression of a disease. For example, treatment can be the diminishment of one or several symptoms of a disorder or complete eradication of a disorder, such as cancer.

As used herein, the term “intermittent” or “intermittently” in the context of administration of Compound A means that the compound is not administered continuously, i.e. not daily, but that there are administration breaks of defined length (days, weeks) between days of administration.

### BRIEF DESCRIPTION OF THE SEVERAL VIEWS OF THE DRAWINGS

FIG. 1: illustrates the XRPD of the crystalline form of Compound A of the present invention obtained from the solvent system IPA/H₂O according to Example 1 herein. The x-axis shows the scattering angle in °2-theta, the y-axis shows the intensity of the scattered X-ray beam in counts of detected photons.

FIG. 2: illustrates the XRPD of the crystalline form of Compound A of the present invention obtained from the solvent system EtOH/H₂O according to Example 2-1 herein. The x-axis shows the scattering angle in °2-theta, the y-axis shows the intensity of the scattered X-ray beam in counts of detected photons.

FIG. 3: illustrates the XRPD of the crystalline form of Compound A of the present invention obtained from the solvent system n-PrOH/H₂O according to Example 2-2 herein. The x-axis shows the scattering angle in °2-theta, the y-axis shows the intensity of the scattered X-ray beam in counts of detected photons.

FIG. 4: illustrates the XRPD of the crystalline form of Compound A of the present invention obtained from ACN/H₂O according to Example 3 herein. The x-axis shows the scattering angle in °2-theta, the y-axis shows the intensity of the scattered X-ray beam in counts of detected photons.

FIG. 5: illustrates the XRPD of the crystalline form of Compound A of the present invention obtained from ACT/H₂O according to Example 4 herein. The x-axis shows the scattering angle in °2-theta, the y-axis shows the intensity of the scattered X-ray beam in counts of detected photons.

FIG. 6: illustrates the XRPDs of the crystalline form of Compound A of the present invention obtained according to the procedure of Example 1 before (top) and after (bottom) 6 months storage at accelerated humidity and temperature stress conditions. The x-axes show the scattering angles in °2-theta. The y-axes show the intensities of the scattered X-ray beam in counts of detected photons.

FIG. 7: illustrates XRPDs of tablet cores pressed from the final blend of formulation F5 of Example 11: placebo (top), tablet containing the crystalline form of Compound A of the present invention obtained according to the procedure of Example 1 with standard run (middle) and low angle slow run (bottom). The x-axes show the scattering angles in °2-theta. The y-axes show the intensities of the scattered X-ray beam in counts of detected photons.

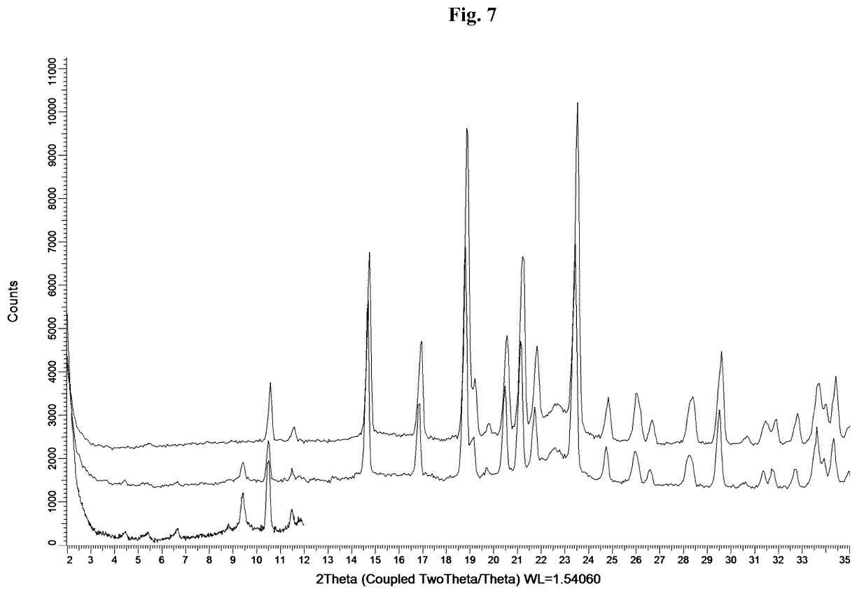

FIG. 8: illustrates the Raman spectrum of the crystalline form of Compound A of the present invention obtained from the solvent system IPA/H₂O according to Example 1 herein. The x-axis shows the wavenumbers in cm⁻¹, the y axis shows the Raman intensities in counts.

FIG. 9: illustrates the Raman spectrum of the crystalline form of Compound A of the present invention obtained from the solvent system ACN/H₂O according to Example 3 herein. The x-axis shows the wavenumbers in cm⁻¹, the y axis shows the Raman intensities in counts.

FIG. 10: illustrates the ¹³C-ssNMR spectrum of the crystalline form of Compound A of the present invention obtained from the solvent system IPA/H₂O according to Example 1 herein. The asterisk denotes a solvent peak originating from IPA. The x-axis shows the chemical shifts in parts per million (ppm).

FIG. 11: illustrates the ¹³C-ssNMR spectrum of the crystalline form of Compound A of the present invention obtained from the solvent system ACN/H₂O according to Example 3 herein. The x-axis shows the chemical shifts in parts per million (ppm).

FIG. 12: illustrates the ¹⁹F-ssNMR spectrum of the crystalline form of Compound A of the present invention obtained from the solvent system IPA/H₂O according to Example 1 herein. The x-axis shows the chemical shifts in parts per million (ppm).

FIG. 13: illustrates the ¹⁹F-ssNMR spectrum of the crystalline form of Compound A of the present invention obtained from the solvent system ACN/H₂O according to Example 3 herein. The x-axis shows the chemical shifts in parts per million (ppm).

FIG. 14: illustrates a gravimetric moisture sorption curve of the crystalline form of Compound A of the present invention obtained from the solvent system IPA/H₂O in the range of from 0 to 90% relative humidity. The x-axis displays the relative humidity in percent (%) measured at a temperature of (25.0±0.1) ° C., the y-axis displays the equilibrium mass change in percent (%) by weight. The sample weight at 0% relative humidity was used as reference weight.

FIG. 15: illustrates a gravimetric moisture sorption curve of the crystalline form of Compound A of the present invention obtained from the solvent system ACN/H₂O in the range of from 0 to 90% relative humidity. The x-axis displays the relative humidity in percent (%) measured at a temperature of (25.0±0.1) ° C., the y-axis displays the equilibrium mass change in percent (%) by weight. The sample weight at 0% relative humidity was used as reference weight.

FIG. 16: illustrates the bioaccessibility profiles of drug products containing the crystalline Compound A of the present invention having different PSDs and amorphous contents. The y-axis shows the bioaccessibility in percent, the x-axis the time in minutes.

FIG. 17: illustrates the concentration profiles of drug products containing the crystalline Compound A of the present invention having different PSDs and amorphous contents. The y-axis shows the concentration in μg/mL, the x-axis the time in minutes.

FIG. 18: illustrates comparative dissolution profiles in 0.05 M sodium phosphate buffer pH 6.8 of granules containing Compound A with 1% (w/w) SLS (top curve, triangles) or without SLS (bottom curve, squares). The y-axis shows the amount of dissolved Compound A in % (w/w), the x-axis shows the time in minutes.

FIG. 19: illustrates comparative dissolution profiles in FaSSGF media pH 1.6 (0-30 min) and FaSSIF media pH 6.5 (30-60 min) of tablet cores containing Compound A with 1% (w/w) SLS (top curve, triangles) or without SLS (bottom curve, squares). The y-axis shows the amount of dissolved Compound A in % (w/w), the x-axis shows the time in minutes.

FIG. 20: illustrates the XRPD of Compound A DIPE solvate obtained from Reference Example 1 herein. The x-axis shows the scattering angle in °2-theta, the y-axis shows the intensity of the scattered X-ray beam in counts of detected photons.

FIG. 21: illustrates the XRPD of Compound A DIPE solvate after open storage under vacuum at 125° C. for 1 day. The x-axis shows the scattering angle in °2-theta, the y-axis shows the intensity of the scattered X-ray beam in counts of detected photons.

FIG. 22: illustrates a comparison of the XRPDs of the crystalline form of Compound A of the present invention obtained from Example 1 (top diffractogram), from Example 2.1 (middle diffractogram) and from Example 2.2 (bottom diffractogram). The x-axis shows the scattering angles in °2-theta. The y-axis shows the intensities of the scattered X-ray beam in counts of detected photons.

FIG. 23: illustrates a comparison of the XRPDs of the crystalline form of Compound A of the present invention obtained from Example 3 (top diffractogram), and from Example 4 (bottom diffractogram). The x-axis shows the scattering angles in °2-theta. The y-axis show the intensities of the scattered X-ray beam in counts of detected photons.

FIG. 24: illustrates a comparison of the XRPDs of the crystalline form of Compound A of the present invention obtained from Example 1 (top diffractogram), and of Compound A DIPE solvate obtained from Reference Example 1 herein (bottom diffractogram). The x-axis shows the scattering angles in °2-theta. The y-axis shows the intensities of the scattered X-ray beam in counts of detected photons.

FIG. 25: illustrates a comparison of the XRPDs of the crystalline form of Compound A of the present invention obtained from Example 3 (top diffractogram), and of Compound A DIPE solvate obtained from Reference Example 1 herein (bottom diffractogram). The x-axis shows the scattering angles in °2-theta. The y-axis shows the intensities of the scattered X-ray beam in counts of detected photons.

### DETAILED DESCRIPTION OF THE INVENTION

In a first aspect, the present invention relates to (2′S,3′S,3a′S,10a′S)-6-chloro-3′-(3-chloro-2-fluorophenyl)-1′-(cyclopropylmethyl)-6′-methyl-2-oxo-1,2,3′,3a′,10′,10a′-hexahydro-1′H-spiro[indole-3,2′-pyrrolo[2′,3′:4,5]pyrrolo[1,2-b]indazole]-7′-carboxylic acid, herein also designated as “Compound A”, of Formula (I)

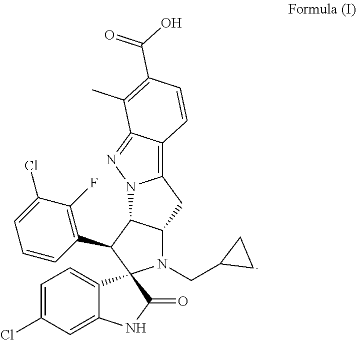 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

in crystalline form.

The crystalline form of Compound A of the present invention may be characterized by analytical methods well known in the field of the pharmaceutical industry for characterizing solids. Such methods comprise but are not limited to XRPD, ssNMR, FTIR spectroscopy, Raman spectroscopy, DSC, TGA and GMS. The crystalline form of Compound A of the present invention may be characterized by one of the aforementioned analytical methods or by combining two or more of them. In particular, the crystalline form of Compound A of the present invention may be characterized by any one of the following embodiments or by combining two or more of the following embodiments.

In one embodiment, the present invention relates to a crystalline form of Compound A characterized by having an XRPD comprising a reflection at 2-theta angles in the range of from 9.2° to 9.6°, when measured at a temperature in the range of from 20 to 30° C. with Cu-Kalpha radiation having a wavelength of 0.15406 nm.

In another embodiment, the present invention relates to a crystalline form of Compound A characterized by having an XRPD comprising a reflection at 2-theta angles in the range of from 9.3° to 9.6°, when measured at a temperature in the range of from 20 to 30° C. with Cu-Kalpha radiation having a wavelength of 0.15406 nm.

In still another embodiment, the present invention relates to a crystalline form of Compound A characterized by having an XRPD comprising a reflection at 2-theta angles in the range of from 9.3° to 9.5°, when measured at a temperature in the range of from 20 to 30° C. with Cu-Kalpha radiation having a wavelength of 0.15406 nm.

In a particular embodiment, the present invention relates to a crystalline form of Compound A characterized by having an XRPD comprising a reflection at 2-theta angles of (9.3±0.2°), (9.4±0.2°) or (9.5°±0.2°), when measured at a temperature in the range of from 20 to 30° C. with Cu-Kalpha radiation having a wavelength of 0.15406 nm.

In yet another embodiment, the present invention relates to a crystalline form of Compound A characterized by having an XRPD comprising a reflection at 2-theta angles in the range of from 9.4° to 9.6°, when measured at a temperature in the range of from 20 to 30° C. with Cu-Kalpha radiation having a wavelength of 0.15406 nm.

In another particular embodiment, the present invention relates to a crystalline form of Compound A characterized by having an XRPD comprising a reflection at 2-theta angles of (9.4±0.2°) or (9.6°±0.2°), when measured at a temperature in the range of from 20 to 30° C. with Cu-Kalpha radiation having a wavelength of 0.15406 nm.

In a specific embodiment, the present invention relates to a crystalline form of Compound A characterized by having an XRPD comprising a reflection at 2-theta angles in the range of from 9.2° to 9.6°, or 9.3° to 9.6°, or 9.3° to 9.5°, or 9.4° to 9.6° or at 2-theta angles of (9.3±0.2°), (9.4±0.2°), (9.5±0.2°) or (9.6±0.2°), when measured at a temperature in the range of from 20 to 30° C. with Cu-Kalpha radiation having a wavelength of 0.15406 nm, wherein said reflection has a relative intensity in height of 100%.

In yet another embodiment, the present invention relates to a crystalline form of Compound A, characterized by having an XRPD comprising a reflection at 2-theta angles in the range of from 4.0° to 4.6°, when measured at a temperature in the range of from 20 to 30° C. with Cu-Kalpha radiation having a wavelength of 0.15406 nm.

In a further embodiment, the present invention relates to a crystalline form of Compound A, characterized by having an XRPD comprising a reflection at 2-theta angles in the range of from 4.1° to 4.5°, when measured at a temperature in the range of from 20 to 30° C. with Cu-Kalpha radiation having a wavelength of 0.15406 nm.

In yet another embodiment, the present invention relates to a crystalline form of Compound A, characterized by having an XRPD comprising a reflection at 2-theta angles in the range of from 4.4° to 4.6°, when measured at a temperature in the range of from 20 to 30° C. with Cu-Kalpha radiation having a wavelength of 0.15406 nm.

In still another embodiment, the present invention relates to a crystalline form of Compound A, characterized by having an XRPD comprising a reflection at 2-theta angles in the range of from 4.4° to 4.5°, when measured at a temperature in the range of from 20 to 30° C. with Cu-Kalpha radiation having a wavelength of 0.15406 nm.

In another embodiment, the present invention relates to a crystalline form of Compound A, characterized by having an XRPD comprising a reflection at 2-theta angles in the range of from 4.1° to 4.2°, when measured at a temperature in the range of from 20 to 30° C. with Cu-Kalpha radiation having a wavelength of 0.15406 nm.

In a particular embodiment, the present invention relates to a crystalline form of Compound A characterized by having an XRPD comprising a reflection at 2-theta angles of (4.1±0.2°), (4.2±0.2°), (4.4±0.2°), or (4.5±0.2), when measured at a temperature in the range of from 20 to 30° C. with Cu-Kalpha radiation having a wavelength of 0.15406 nm.

In one embodiment, the present invention relates to a crystalline form of Compound A characterized by having an XRPD comprising a reflection at 2-theta angles in the range of from 4.0° to 4.6° and a reflection at 2-theta angles in the range of from 9.2° to 9.6°, when measured at a temperature in the range of from 20 to 30° C. with Cu-Kalpha radiation having a wavelength of 0.15406 nm.

In another embodiment, the present invention relates to a crystalline form of Compound A characterized by having an XRPD comprising a reflection at 2-theta angles in the range of from 4.1° to 4.5° and a reflection at 2-theta angles in the range of from 9.30 to 9.6°, when measured at a temperature in the range of from 20 to 30° C. with Cu-Kalpha radiation having a wavelength of 0.15406 nm.

In another embodiment, the present invention relates to a crystalline form of Compound A characterized by having an XRPD comprising a reflection at 2-theta angles in the range of from 4.4° to 4.6° and a reflection at 2-theta angles in the range of from 9.3° to 9.6°, when measured at a temperature in the range of from 20 to 30° C. with Cu-Kalpha radiation having a wavelength of 0.15406 nm.

In a further embodiment, the present invention relates to a crystalline form of Compound A characterized by having an XRPD comprising a reflection at 2-theta angles in the range of from 4.4° to 4.5° and a reflection at 2-theta angles in the range of from 9.30 to 9.5°, when measured at a temperature in the range of from 20 to 30° C. with Cu-Kalpha radiation having a wavelength of 0.15406 nm.

In another embodiment, the present invention relates to a crystalline form of Compound A characterized by having an XRPD comprising a reflection at 2-theta angles in the range of from 4.1° to 4.2° and a reflection at 2-theta angles in the range of from 9.4° to 9.6°, when measured at a temperature in the range of from 20 to 30° C. with Cu-Kalpha radiation having a wavelength of 0.15406 nm.

In still another embodiment, the present invention relates to a crystalline form of Compound A characterized by having an XRPD comprising a reflection at 2-theta angles of (4.1±0.2°) and (9.4±0.2°), when measured at a temperature in the range of from 20 to 30° C. with Cu-Kalpha radiation having a wavelength of 0.15406 nm.

In still another embodiment, the present invention relates to a crystalline form of Compound A characterized by having an XRPD comprising a reflection at 2-theta angles of (4.2±0.2°) and (9.6±0.2°), when measured at a temperature in the range of from 20 to 30° C. with Cu-Kalpha radiation having a wavelength of 0.15406 nm.

In yet another embodiment, the present invention relates to a crystalline form of Compound A characterized by having an XRPD comprising a reflection at 2-theta angles of (4.4±0.2°) or (4.5±0.2°) and a reflection at 2-theta angles of (9.3±0.2°), (9.4±0.2°) or (9.5±0.2°), when measured at a temperature in the range of from 20 to 30° C. with Cu-Kalpha radiation having a wavelength of 0.15406 nm.

In another preferred embodiment, the present invention relates to a crystalline form of Compound A characterized by having an XRPD comprising reflections at 2-theta angles of (4.4±0.2°) and (9.3±0.2°), when measured at a temperature in the range of from 20 to 30° C. with Cu-Kalpha radiation having a wavelength of 0.15406 nm.

In another embodiment, the present invention relates to a crystalline form of Compound A characterized by having an XRPD comprising reflections at 2-theta angles of (4.5±0.2°) and (9.4±0.2°), when measured at a temperature in the range of from 20 to 30° C. with Cu-Kalpha radiation having a wavelength of 0.15406 nm.

In still another embodiment, the present invention relates to a crystalline form of Compound A characterized by having an XRPD comprising reflections at 2-theta angles of (4.5±0.2°) and (9.5±0.2°), when measured at a temperature in the range of from 20 to 30° C. with Cu-Kalpha radiation having a wavelength of 0.15406 nm.

In a further embodiment, the present invention relates to a crystalline form of Compound A characterized by having an XRPD comprising a reflection at 2-theta angles in the range of from 6.5° to 6.8°, when measured at a temperature in the range of from 20 to 30° C. with Cu-Kalpha radiation having a wavelength of 0.15406 nm.

In another embodiment, the present invention relates to a crystalline form of Compound A characterized by having an XRPD comprising a reflection at 2-theta angles in the range of from 6.6° to 6.8°, when measured at a temperature in the range of from 20 to 30° C. with Cu-Kalpha radiation having a wavelength of 0.15406 nm.

In still another embodiment, the present invention relates to a crystalline form of Compound A characterized by having an XRPD comprising a reflection at 2-theta angles in the range of from 6.6° to 6.7°, when measured at a temperature in the range of from 20 to 30° C. with Cu-Kalpha radiation having a wavelength of 0.15406 nm.

In a particular embodiment, the present invention relates to a crystalline form of Compound A characterized by having an XRPD comprising a reflection at 2-theta angles of (6.5±0.2°), (6.6±0.2°) or (6.7±0.2°), when measured at a temperature in the range of from 20 to 30° C. with Cu-Kalpha radiation having a wavelength of 0.15406 nm.

In one embodiment, the present invention relates to a crystalline form of Compound A characterized by having an XRPD comprising a reflection at 2-theta angles in the range of from 4.0° to 4.6°, of from 6.5° to 6.8°, and of from 9.2° to 9.6°, when measured at a temperature in the range of from 20 to 30° C. with Cu-Kalpha radiation having a wavelength of 0.15406 nm.

In a further embodiment, the present invention relates to a crystalline form of Compound A characterized by having an XRPD comprising a reflection at 2-theta angles in the range of from 4.1° to 4.5°, of from 6.6° to 6.7°, and of from 9.3° to 9.6°, when measured at a temperature in the range of from 20 to 30° C. with Cu-Kalpha radiation having a wavelength of 0.15406 nm.

In another embodiment, the present invention relates to a crystalline form of Compound A characterized by having an XRPD comprising a reflection at 2-theta angles in the range of from 4.4° to 4.6°, of from 6.6° to 6.8°, and of from 9.3° to 9.6°, when measured at a temperature in the range of from 20 to 30° C. with Cu-Kalpha radiation having a wavelength of 0.15406 nm.

In a further embodiment, the present invention relates to a crystalline form of Compound A characterized by having an XRPD comprising a reflection at 2-theta angles in the range of from 4.4° to 4.5°, of from 6.6° to 6.7°, and of from 9.3° to 9.6°, when measured at a temperature in the range of from 20 to 30° C. with Cu-Kalpha radiation having a wavelength of 0.15406 nm.

In still a further embodiment, the present invention relates to a crystalline form of Compound A characterized by having an XRPD comprising a reflection at 2-theta angles in the range of from 4.1° to 4.2°, of from 6.6° to 6.7°, and of from 9.4° to 9.6°, when measured at a temperature in the range of from 20 to 30° C. with Cu-Kalpha radiation having a wavelength of 0.15406 nm.

In yet another embodiment, the present invention relates to a crystalline form of Compound A characterized by having an XRPD comprising a reflection at 2-theta angles of (4.4±0.2°) or (4.5±0.2°), a reflection at 2-theta angles of (6.6±0.2°) or (6.7±0.2°), and a reflection at 2-theta angles of (9.3±0.2°), (9.4±0.2°) or (9.5±0.2°), when measured at a temperature in the range of from 20 to 30° C. with Cu-Kalpha radiation having a wavelength of 0.15406 nm.

In still another embodiment, the present invention relates to a crystalline form of Compound A characterized by having an XRPD comprising reflections at 2-theta angles of:
(4.4±0.2°), (6.6±0.2°) and (9.3±0.2°); or
(4.4±0.2°), (6.6±0.2°), (8.7±0.2°) and (9.3±0.2°); or
(4.4±0.2)°, (6.6±0.2)°, (8.7±0.2°), (9.3±0.2°) and (11.9±0.2°); or
(4.4±0.2)°, (5.1±0.2)°, (6.6±0.2)°, (8.7±0.2)°, (9.3±0.2°) and (11.9±0.2°); or
(4.4±0.2)°, (5.1±0.2)°, (6.6±0.2)°, (8.7±0.2)°, (9.3±0.2°), (11.9±0.2°) and (13.2±0.2°); or
(4.4±0.2)°, (5.1±0.2)°, (6.6±0.2°), (8.7±0.2°), (9.3±0.2°), (11.9±0.2°), (13.2±0.2°) and (14.2±0.2°); or
(4.4±0.2°), (5.1±0.2°), (6.6±0.2°), (8.7±0.2°), (9.3±0.2°), (11.9±0.2°), (13.2±0.2°), (14.2±0.2°) and (19.9±0.2°); or
(4.4±0.2°), (5.1±0.2°), (6.6±0.2°), (8.7±0.2°), (9.3±0.2°), (11.9±0.2°), (13.2±0.2°), (14.2±0.2°), (19.9±0.2°) and (23.0±0.2°);
when measured at a temperature in the range of from 20 to 30° C. with Cu-Kalpha radiation having a wavelength of 0.15406 nm.

In another embodiment, the invention relates to a crystalline form of Compound A characterized by having an XRPD comprising reflections at 2-Theta angles of (4.4±0.2°), (8.7±0.2°), (9.3±0.2°), (11.9±0.2°), (13.2±0.2°), (13.8±0.2°), (14.2±0.2°), (18.6±0.2°), (19.9±0.2°) and (23.0±0.2°), when measured at a temperature in the range of from 20 to 30° C. with Cu-Kalpha radiation having a wavelength of 0.15406 nm.

In a further embodiment, the present invention relates to a crystalline form of Compound A characterized by having an XRPD comprising reflections at 2-Theta angles of:
(4.4±0.1)°, (6.6±0.1°) and (9.3±0.1°); or
(4.4±0.1)°, (6.6±0.1)°, (8.7±0.1°) and (9.3±0.1°); or
(4.4±0.1)°, (6.6±0.1)°, (8.7±0.1°), (9.3±0.1°) and (11.9±0.1°); or
(4.4±0.1)°, (5.1±0.1)°, (6.6±0.1)°, (8.7±0.1)°, (9.3±0.1°) and (11.9±0.1°); or
(4.4±0.1)°, (5.1±0.1)°, (6.6±0.1)°, (8.7±0.1)°, (9.3±0.1°), (11.9±0.1°) and (13.2±0.1°); or
(4.4±0.1)°, (5.1±0.1)°, (6.6±0.1°), (8.7±0.1°), (9.3±0.1°), (11.9±0.1°), (13.2±0.1°) and (14.2±0.1°); or
(4.4±0.1°), (5.1±0.1°), (6.6±0.1°), (8.7±0.1°), (9.3±0.1°), (11.9±0.1°), (13.2±0.1°), (14.2±0.1°) and (19.9±0.1°); or
(4.4±0.1°), (5.1±0.1°), (6.6±0.1°), (8.7±0.1°), (9.3±0.1°), (11.9±0.1°), (13.2±0.1°), (14.2±0.1°), (19.9±0.1°) and (23.0±0.1°);
when measured at a temperature in the range of from 20 to 30° C. with Cu-Kalpha radiation having a wavelength of 0.15406 nm.

In yet another embodiment, the invention relates to a crystalline form of Compound A characterized by having an XRPD comprising reflections at 2-Theta angles of (4.4±0.1°), (8.7±0.1°), (9.3±0.1°), (11.9±0.1°), (13.2±0.1°), (13.8±0.1°), (14.2±0.1°), (18.6±0.1°), (19.9±0.1°) and (23.0±0.1°), when measured at a temperature in the range of from 20 to 30° C. with Cu-Kalpha radiation having a wavelength of 0.15406 nm.

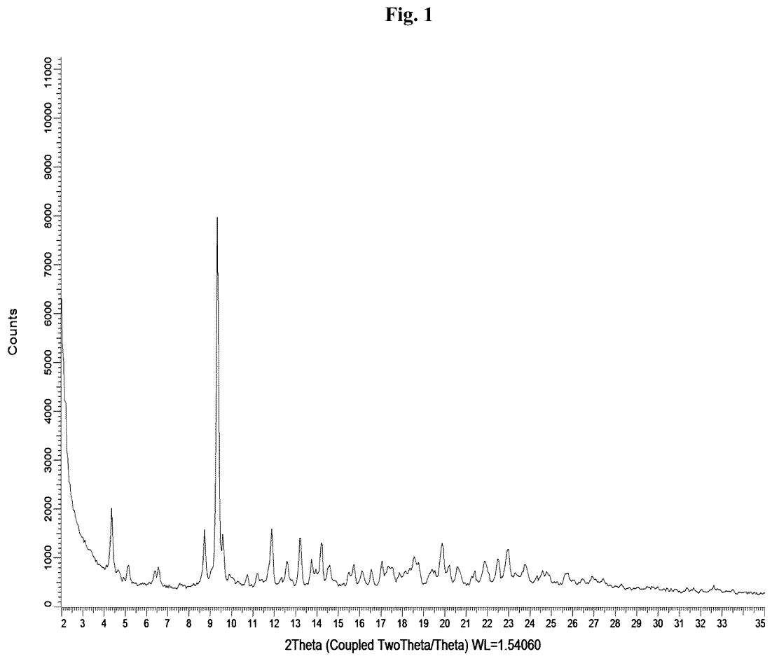

In a further embodiment, the invention relates to a crystalline form of Compound A characterized by having an XRPD essentially the same as shown in FIG. 1, when measured at a temperature in the range of from 20 to 30° C. with Cu-Kalpharadiation having a wavelength of 0.15406 nm.

In another embodiment, the invention relates to a crystalline form of Compound A characterized by having a Raman spectrum comprising a peak at wavenumbers of (1680±2) cm⁻¹, when measured at a temperature in the range of from 20 to 30° C. and a wavelength of 830 nm.

In yet another embodiment, the invention relates to a crystalline form of Compound A characterized by having a Raman spectrum comprising peaks at wavenumbers of:
(1680±2) cm⁻¹ and (1251±2) cm⁻¹; or
(1163±2) cm⁻¹, (1251±2) cm⁻¹ and (1680±2) cm⁻¹; or
(1163±2) cm⁻¹, (1251±2) cm⁻¹, (1621±2) cm⁻¹, and (1680±2) cm⁻¹; or
(1163±2) cm⁻¹, (1251±2) cm⁻¹, (1430±2) cm⁻¹, (1621±2) cm−¹ and (1680±2) cm⁻¹; or
(1163±2) cm⁻¹, (1251±2) cm⁻¹, (1385±2) cm−¹, (1430±2) cm⁻¹ (1621±2) cm⁻¹ and (1680±2) cm⁻¹;
when measured at a temperature in the range of from 20 to 30° C. and a wavelength of 830 nm.

In a further embodiment, the invention relates to a crystalline form of Compound A characterized by having a ¹³C-ssNMR spectrum comprising peaks at (18.6±0.3) ppm and/or (176.4±0.3) ppm.

In a further embodiment, the invention relates to a crystalline form of Compound A characterized by having an ¹⁹F-ssNMR spectrum comprising peaks at:
(−117.5±0.5) ppm and/or (−120.3±0.5) ppm; or
(−114.6±0.5) ppm, (−117.5±0.5) ppm and (−120.3±0.5) ppm; or
(−112.2±0.5) ppm, (−114.6±0.5) ppm, (−117.5±0.5) ppm and (−120.3±0.5) ppm;

In still another embodiment, the present invention relates to a crystalline form of Compound A characterized by having an XRPD comprising reflections at 2-theta angles of:
(4.2±0.2°), (6.3±0.2°) and (9.6±0.2°); or
(4.2±0.2°), (6.3±0.2)°, (9.6±0.2°) and (13.4±0.2°); or
(4.2±0.2)°, (6.3±0.2)°, (9.6±0.2°), (10.3±0.2°) and (13.4±0.2°); or
(4.2±0.2)°, (5.3±0.2)°, (6.3±0.2)°, (9.6±0.2)°, (10.3±0.2°) and (13.4±0.2°); or
(4.2±0.2)°, (5.3±0.2)°, (6.3±0.2)°, (9.6±0.2)°, (10.3±0.2°), (12.0±0.2°) and (13.4±0.2°); or
(4.2±0.2)°, (5.3±0.2°), (6.3±0.2°), (9.6±0.2°), (10.3±0.2°), (11.2±0.2°), (12.0±0.2°) and (13.4±0.2°); or
(4.2±0.2°), (5.3±0.2°), (6.3±0.2°), (7.6±0.2°), (9.6±0.2°), (10.3±0.2°), (11.2±0.2°), (12.0±0.2°) and (13.4±0.2°); or
(4.2±0.2°), (5.3±0.2°), (6.3±0.2°), (7.6±0.2°), (9.6±0.2°), (10.3±0.2°), (11.2±0.2°), (12.0±0.2°), (13.4±0.2°) and (15.5±0.2°);
when measured at a temperature in the range of from 20 to 30° C. with Cu-Kalpha radiation having a wavelength of 0.15406 nm.

In another embodiment, the invention relates to a crystalline form of Compound A characterized by having an XRPD comprising reflections at 2-Theta angles of (4.2±0.2°), (6.3±0.2°), (9.6±0.2°), (10.3±0.2°), (12.0±0.2°), (13.4±0.2°), (13.8±0.2°), (15.5±0.2°), (19.0±0.2°) and (19.4±0.2°), when measured at a temperature in the range of from 20 to 30° C. with Cu-Kalpha radiation having a wavelength of 0.15406 nm.

In a further embodiment, the present invention relates to a crystalline form of Compound A characterized by having an XRPD comprising reflections at 2-Theta angles of:
(4.2±0.1°), (6.3±0.1°) and (9.6±0.1°); or
(4.2±0.1°), (6.3±0.1°), (9.6±0.1°) and (13.4±0.1°); or
(4.2±0.1°), (6.3±0.1°), (9.6±0.1°), (10.3±0.1°) and (13.4±0.1°); or (4.2±0.1°), (5.3±0.1°), (6.3±0.1°), (9.6±0.1°), (10.3±0.1°) and (13.4±0.1°); or
(4.2±0.1°), (5.3±0.1°), (6.3±0.1°), (9.6±0.1°), (10.3±0.1°), (12.0±0.1°) and (13.4±0.1°); or
(4.2±0.1°), (5.3±0.1°), (6.3±0.1°), (9.6±0.1°), (10.3±0.1°), (11.2±0.1°), (12.0±0.1°) and (13.4±0.1°); or
(4.2±0.1°), (5.3±0.1°), (6.3±0.1°), (7.6±0.1°), (9.6±0.1°), (10.3±0.1°), (11.2±0.1°), (12.0±0.1°) and (13.4±0.1°); or
(4.2±0.1°), (5.3±0.1°), (6.3±0.1°), (7.6±0.1°), (9.6±0.1°), (10.3±0.1°), (11.2±0.1°), (12.0±0.1°), (13.4±0.1°) and (15.5±0.1°);
when measured at a temperature in the range of from 20 to 30° C. with Cu-Kalpha radiation having a wavelength of 0.15406 nm.

In yet another embodiment, the invention relates to a crystalline form of Compound A characterized by having an XRPD comprising reflections at 2-Theta angles of (4.2±0.1°), (6.3±0.1°), (9.6±0.1°), (10.3±0.1°), (12.0±0.1°), (13.4±0.1°), (13.8±0.1°), (15.5±0.1°), (19.0±0.1°) and (19.4±0.1°), when measured at a temperature in the range of from 20 to 30° C. with Cu-Kalpha radiation having a wavelength of 0.15406 nm.

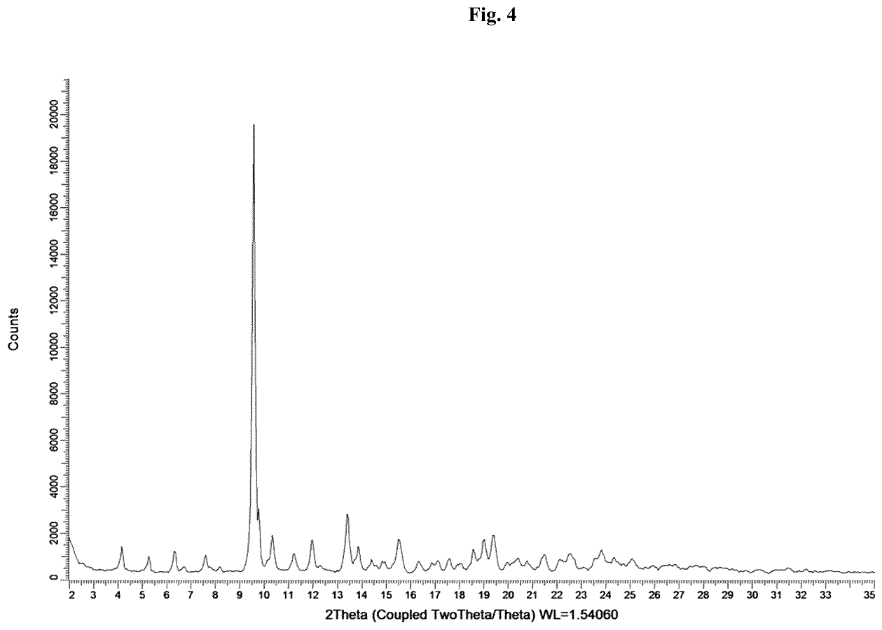

In a further embodiment, the invention relates to a crystalline form of Compound A characterized by having an XRPD essentially the same as shown in FIG. 4, when measured at a temperature in the range of from 20 to 30° C. with Cu-Kalpharadiation having a wavelength of 0.15406 nm.

In a further embodiment, the invention relates to a crystalline form of Compound A characterized by having a Raman spectrum comprising a peak at wavenumbers of (1732±2) cm⁻¹, when measured at a temperature in the range of from 20 to 30° C. and a wavelength of 830 nm.

In yet another embodiment, the invention relates to a crystalline form of Compound A characterized by having a Raman spectrum comprising peaks at wavenumbers of:
(1675±2) cm⁻¹ and (1732±2) cm⁻¹; or
(1675±2) cm⁻¹, (1699±2) cm⁻¹ and (1732±2) cm⁻¹; or
(1251±2) cm−¹, (1675±2) cm−¹ (1699±2) cm−¹ and (1732±2) cm⁻¹; or
(1164±2) cm−¹, (1251±2) cm−¹, (1675±2) cm−¹, (1699±2) cm−¹ and (1732±2) cm⁻¹; or
(1164±2) cm⁻¹, (1251±2) cm⁻¹, (1621±2) cm−¹, (1675±2) cm⁻¹, (1699±2) cm−¹ and (1732±2) cm−¹; or
(1164±2) cm−¹, (1251±2) cm−¹, (1430±2) cm−¹, (1621±2) cm−¹, (1675±2) cm−¹, (1699±2) cm−¹ and (1732±2) cm−¹; or
(1164±2) cm−¹, (1251±2) cm−¹, (1385±2) cm−¹, (1430±2) cm−¹, (1621±2) cm−¹, (1675±2) cm−¹, (1699±2) cm−¹ and (1732±2) cm⁻¹;
when measured at a temperature in the range of from 20 to 30° C. and a wavelength of 830 nm.

In another embodiment, the invention relates to a crystalline form of Compound A characterized by having a ¹³C-ssNMR spectrum comprising peaks at
(16.4±0.3) ppm and/or (112.4±0.3) ppm; or
(16.4±0.3) ppm, (112.4±0.3) ppm and (174.7±0.3) ppm; or
(16.4±0.3) ppm, (112.4±0.3) ppm, (117.1±0.3) ppm and (174.7±0.3) ppm; or
(16.4±0.3) ppm, (112.4±0.3) ppm, (117.1±0.3) ppm, (126.1±0.3) ppm and (174.7±0.3) ppm; or
(16.4±0.3) ppm, (112.4±0.3) ppm, (117.1±0.3) ppm, (121.3±0.3) ppm, (126.1±0.3) ppm, and (174.7±0.3) ppm;

In a further embodiment, the invention relates to a crystalline form of Compound A characterized by having an ¹⁹F-ssNMR spectrum comprising peaks at:
(−116.2±0.5) ppm and/or (−118.6±0.5) ppm; or
(−116.2±0.5) ppm, (−118.6±0.5) ppm and (−121.0±0.5) ppm; or
(−116.2±0.5) ppm, (−118.6±0.5) ppm, (−120.2±0.5) ppm and (−121.0±0.5) ppm; or
(−116.2±0.5) ppm, (−117.9±0.5) ppm, (−118.6±0.5) ppm, (−120.2±0.5) ppm and (−121.0±0.5) ppm; or
(−116.2±0.5) ppm, (−117.9±0.5) ppm, (−118.6±0.5) ppm, (−120.2±0.5) ppm, (−121.0±0.5) ppm and (−122.1±0.5 ppm);

In a further embodiment, the invention relates to a crystalline form of Compound A characterized by a particle size distribution having a D90 value of not more than 100 μm, preferably of not more than 80 μm, even more preferably of not more than 65 μm and most preferably of not more than 45 μm, as determined by laser diffraction (He—Ne-laser) with a wet disperser.

In one embodiment, the invention relates to a crystalline form of Compound A characterized by a particle size distribution having a D90 value in the range of from 40 to 100 μm, preferably of from 40 to 80 μm, even more preferably of from 40 to 65 μm, as determined by laser diffraction (He—Ne-laser) with a wet disperser.

In another embodiment, the invention relates to a crystalline form of Compound A characterized by a particle size distribution having a D50 value of not more than 30 μm, preferably of not more than 25 μm, even more preferably of not more than 20 μm and most preferably of not more than 15 μm, as determined by laser diffraction (He—Ne-laser) with a wet disperser.

In one embodiment, the invention relates to a crystalline form of Compound A characterized by a particle size distribution having a D50 value in the range of from 10 to 30 μm, preferably of from 10 to 25 μm, more preferably of from 10 to 20 μm and most preferably of from 10 to 15 μm, as determined by laser diffraction (He—Ne-laser) with a wet disperser.

In still another embodiment, the invention relates to a crystalline form of Compound A characterized by a particle size distribution having a D10 value of not more than 10 μm, preferably of not more than 5 μm, as determined by laser diffraction (He—Ne-laser) with a wet disperser.

In yet another embodiment, the invention relates to a crystalline form of Compound A characterized by a particle size distribution having a D10 value in the range of from 1 to 10 μm, preferably of from 1 to 5 μm, as determined by laser diffraction (He—Ne-laser) with a wet disperser.

In still another embodiment, the invention relates to a crystalline form of Compound A characterized by having an organic solvent content of at most 5000 ppm, preferably of at most 3000 ppm, more preferably at most 410 ppm.

In a specific embodiment, the invention relates to a crystalline form of Compound A having an acetone content, an ethanol content, and/or a 2-propanol content of at most 5000 ppm. In another specific embodiment, the invention relates to a crystalline form of Compound A having an acetonitrile content of not more than 410 ppm.

The organic solvent content may be determined by means well known to the skilled artisan e.g. by gas chromatography or by NMR-spectroscopy in solution or in solid-state.

### Compositions of Compound a Comprising the Crystalline Form of Compound a of the Invention

In another aspect, the present invention relates to a composition of Compound A comprising the crystalline form of Compound A of the present invention as defined in the above-described aspect and corresponding embodiments.

In one embodiment, the present invention relates to a composition comprising the crystalline form of Compound A of the present invention, wherein said composition comprises at most about 50% (w/w), 40% (w/w), 35% (w/w), 33% (w/w), 30% (w/w), 25% (w/w), 20% (w/w) or 18% (w/w), preferably at most about 15% (w/w) or 12% (w/w), more preferably at most about 10% (w/w) and most preferably at most about 9, 8, 7, 6, 5, 4, 3, 2 or 1% (w/w) of any solid-state form other than the crystalline form of Compound A of the present invention, based on the weight of the composition. In a preferred embodiment, said composition is essentially free of any solid-state form other than the crystalline form of Compound A of the present invention. Preferably, another solid-state form of Compound A is amorphous Compound A.

In another embodiment, the present invention relates to a composition comprising the crystalline form of Compound A of the present invention, wherein said composition comprises about 5 to 30% (w/w), preferably about 10 to 30% (w/w), even more preferably about 10-25% w/w of any other solid-state form other than the crystalline form of Compound A of the present invention, based on the weight of the composition. Preferably, another solid-state form of Compound A is amorphous.

In an alternative embodiment, the invention relates to a composition comprising the crystalline form of Compound A of the present invention, wherein the crystalline form of Compound A is present in an amount of at least about 50% (w/w), 60% (w/w), 65% (w/w), 67% (w/w), 70% (w/w), 75% (w/w), 80% (w/w) or 82% (w/w), preferably at least about 85% (w/w) or 88% (w/w), more preferably at least about 90% (w/w), including at least about 90, 91, 92, 93, 94, 95, 96, 97, 98 or 99% (w/w), and also including equal to about 100% (w/w), based on the weight of the composition. The remaining material may comprise other solid-state form(s) of Compound A such as amorphous Compound A, and/or reaction impurities and/or processing impurities arising from the preparation of the composition but excluding any pharmaceutically acceptable excipients. Preferably, the remaining material is amorphous Compound A.

In another embodiment, the present invention relates to a composition comprising the crystalline form of Compound A of the present invention, wherein said composition is characterized by having an XRPD comprising a reflection at 2-theta angles in the range of from 9.2° to 9.6°, when measured at a temperature in the range of from 20 to 30° C. with Cu-Kalpha radiation having a wavelength of 0.15406 nm.

In another embodiment, the present invention relates to a composition comprising the crystalline form of Compound A of the present invention, wherein said composition is characterized by having an XRPD comprising a reflection at 2-theta angles in the range of from 9.3° to 9.6°, when measured at a temperature in the range of from 20 to 30° C. with Cu-Kalpha radiation having a wavelength of 0.15406 nm.

In another embodiment, the present invention relates to a composition comprising the crystalline form of Compound A of the present invention, wherein said composition is characterized by having an XRPD comprising a reflection at 2-theta angles in the range of from 9.3° to 9.5°, when measured at a temperature in the range of from 20 to 30° C. with Cu-Kalpha radiation having a wavelength of 0.15406 nm.

In a particular embodiment, the present invention relates to a composition comprising the crystalline form of Compound A of the present invention, wherein said composition is characterized by having an XRPD comprising a reflection at 2-theta angles of (9.3±0.2°), (9.4±0.2°), or (9.5°±0.2°), when measured at a temperature in the range of from 20 to 30° C. with Cu-Kalpha radiation having a wavelength of 0.15406 nm.

In yet another embodiment, the present invention relates to a composition comprising the crystalline form of Compound A of the present invention, wherein said composition is characterized by having an XRPD comprising a reflection at 2-theta angles in the range of from 9.4° to 9.6°, when measured at a temperature in the range of from 20 to 30° C. with Cu-Kalpha radiation having a wavelength of 0.15406 nm.

In another particular embodiment, the present invention relates to a composition comprising the crystalline form of Compound A of the present invention, wherein said composition is characterized by having an XRPD comprising a reflection at 2-theta angles of (9.4±0.2°) or (9.6°±0.2°), when measured at a temperature in the range of from 20 to 30° C. with Cu-Kalpha radiation having a wavelength of 0.15406 nm.

In yet another embodiment, the present invention relates to a composition comprising the crystalline form of Compound A of the present invention, wherein said composition is characterized by having an XRPD comprising a reflection at 2-theta angles in the range of from 4.0° to 4.6°, when measured at a temperature in the range of from 20 to 30° C. with Cu-Kalpha radiation having a wavelength of 0.15419 nm.

In still another embodiment, the present invention relates to a composition comprising the crystalline form of Compound A of the present invention, wherein said composition is characterized by having an XRPD comprising a reflection at 2-theta angles in the range of from 4.1° to 4.5°, when measured at a temperature in the range of from 20 to 30° C. with Cu-Kalpha radiation having a wavelength of 0.15406 nm.

In yet another embodiment, the present invention relates to a composition comprising the crystalline form of Compound A of the present invention, wherein said composition is characterized by having an XRPD comprising a reflection at 2-theta angles in the range of from 4.4° to 4.6°, when measured at a temperature in the range of from 20 to 30° C. with Cu-Kalpha radiation having a wavelength of 0.15406 nm.

In still another embodiment, the present invention relates to a composition comprising the crystalline form of Compound A of the present invention, wherein said composition is characterized by having an XRPD comprising a reflection at 2-theta angles in the range of from 4.4° to 4.5°, when measured at a temperature in the range of from 20 to 30° C. with Cu-Kalpha radiation having a wavelength of 0.15406 nm.

In another embodiment, the present invention relates to a composition comprising the crystalline form of Compound A of the present invention, wherein said composition is characterized by having an XRPD comprising a reflection at 2-theta angles in the range of from 4.1° to 4.2°, when measured at a temperature in the range of from 20 to 30° C. with Cu-Kalpha radiation having a wavelength of 0.15406 nm.

In a particular embodiment, the present invention relates to a composition comprising the crystalline form of Compound A of the present invention, wherein said composition is characterized by having an XRPD comprising a reflection at 2-theta angles of (4.1±0.2°), (4.2±0.2°), (4.4±0.2°), or (4.5±0.2), when measured at a temperature in the range of from 20 to 30° C. with Cu-Kalpha radiation having a wavelength of 0.15406 nm.

In one embodiment, the present invention relates to a composition comprising the crystalline form of Compound A of the present invention, wherein said composition is characterized by having an XRPD comprising a reflection at 2-theta angles in the range of from 4.0° to 4.6° and in the range of from 9.2° to 9.6°, when measured at a temperature in the range of from 20 to 30° C. with Cu-Kalpha radiation having a wavelength of 0.15406 nm.

In another embodiment, the present invention relates to a composition comprising the crystalline form of Compound A of the present invention, wherein said composition is characterized by having an XRPD comprising a reflection at 2-theta angles in the range of from 4.1° to 4.5° and a reflection at 2-theta angles in the range of from 9.30 to 9.6°, when measured at a temperature in the range of from 20 to 30° C. with Cu-Kalpha radiation having a wavelength of 0.15406 nm.

In another embodiment, the present invention relates to a composition comprising the crystalline form of Compound A of the present invention, wherein said composition is characterized by having an XRPD comprising a reflection at 2-theta angles in the range of from 4.4° to 4.6° and in the range of from 9.3° to 9.6°, when measured at a temperature in the range of from 20 to 30° C. with Cu-Kalpha radiation having a wavelength of 0.15406 nm.

In a further embodiment, the present invention relates to a composition comprising the crystalline form of Compound A of the present invention, wherein said composition is characterized by having an XRPD comprising a reflection at 2-theta angles in the range of from 4.4° to 4.5° and a reflection at 2-theta angles in the range of from 9.30 to 9.50, when measured at a temperature in the range of from 20 to 30° C. with Cu-Kalpha radiation having a wavelength of 0.15406 nm.

In another embodiment, the present invention relates to a composition comprising the crystalline form of Compound A of the present invention, wherein said composition is characterized by having an XRPD comprising a reflection at 2-theta angles in the range of from 4.1° to 4.2° and a reflection at 2-theta angles in the range of from 9.4° to 9.6°, when measured at a temperature in the range of from 20 to 30° C. with Cu-Kalpha radiation having a wavelength of 0.15406 nm.

In still another embodiment, the present invention relates to a composition comprising the crystalline form of Compound A of the present invention, wherein said composition is characterized by having an XRPD comprising a reflection at 2-theta angles of (4.1±0.2°) and (9.4±0.2°), when measured at a temperature in the range of from 20 to 30° C. with Cu-Kalpha radiation having a wavelength of 0.15406 nm.

In still another embodiment, the present invention relates to a composition comprising the crystalline form of Compound A of the present invention, wherein said composition is characterized by having an XRPD comprising a reflection at 2-theta angles of (4.2±0.2°) and (9.6±0.2°), when measured at a temperature in the range of from 20 to 30° C. with Cu-Kalpha radiation having a wavelength of 0.15406 nm.

In yet another embodiment, the present invention relates to a composition comprising the crystalline form of Compound A of the present invention, wherein said composition is characterized by having an XRPD comprising a reflection at 2-theta angles of (4.4±0.2°) or (4.5±0.2°) and a reflection at 2-theta angles of (9.3±0.2°), (9.4±0.2°) or (9.5±0.2°), when measured at a temperature in the range of from 20 to 30° C. with Cu-Kalpha radiation having a wavelength of 0.15406 nm.

In another preferred embodiment, the present invention relates to a composition comprising the crystalline form of Compound A of the present invention, wherein said composition is characterized by having an XRPD comprising reflections at 2-theta angles of (4.4±0.2°) and (9.3±0.2°), when measured at a temperature in the range of from 20 to 30° C. with Cu-Kalpha radiation having a wavelength of 0.15406 nm.

In another preferred embodiment, the present invention relates to a composition comprising the crystalline form of Compound A of the present invention, wherein said composition is characterized by having an XRPD comprising reflections at 2-theta angles of (4.5±0.2°) and (9.4±0.2°), when measured at a temperature in the range of from 20 to 30° C. with Cu-Kalpha radiation having a wavelength of 0.15406 nm.

In still another preferred embodiment, the present invention relates to a composition comprising the crystalline form of Compound A of the present invention, wherein said composition is characterized by having an XRPD comprising reflections at 2-theta angles of (4.5±0.2°) and (9.5±0.2°), when measured at a temperature in the range of from 20 to 30° C. with Cu-Kalpha radiation having a wavelength of 0.15406 nm.

In a further embodiment, the present invention relates to a composition comprising the crystalline form of Compound A of the present invention, wherein said composition is characterized by having an XRPD comprising a reflection at 2-theta angles in the range of from 6.5° to 6.8°, when measured at a temperature in the range of from 20 to 30° C. with Cu-Kalpha radiation having a wavelength of 0.15406 nm.

In another embodiment, the present invention relates to a composition comprising the crystalline form of Compound A of the present invention, wherein said composition is characterized by having an XRPD comprising a reflection at 2-theta angles in the range of from 6.6° to 6.8°, when measured at a temperature in the range of from 20 to 30° C. with Cu-Kalpha radiation having a wavelength of 0.15406 nm.

In another embodiment, the present invention relates to a composition comprising the crystalline form of Compound A of the present invention, wherein said composition is characterized by having an XRPD comprising a reflection at 2-theta angles in the range of from 6.6° to 6.7°, when measured at a temperature in the range of from 20 to 30° C. with Cu-Kalpha radiation having a wavelength of 0.15406 nm.

In a particular embodiment, the present invention relates to a composition comprising the crystalline form of Compound A of the present invention, wherein said composition is characterized by having an XRPD comprising a reflection at 2-theta angles of (6.5±0.2°), (6.6±0.2°), or (6.7±0.2°), when measured at a temperature in the range of from 20 to 30° C. with Cu-Kalpha radiation having a wavelength of 0.15406 nm.

In one embodiment, the present invention relates to a composition comprising the crystalline form of Compound A of the present invention, wherein said composition is characterized by having an XRPD comprising a reflection at 2-theta angles in the range of from 4.0° to 4.6°, in the range of from 6.5° to 6.8°, and in the range of from 9.2° to 9.6°, when measured at a temperature in the range of from 20 to 30° C. with Cu-Kalpha radiation having a wavelength of 0.15406 nm.

In a further embodiment, the present invention relates to a composition comprising the crystalline form of Compound A of the present invention, wherein said composition is characterized by having an XRPD comprising a reflection at 2-theta angles in the range of from 4.1° to 4.5°, in the range of from 6.6° to 6.7°, and in the range of from 9.3° to 9.6°, when measured at a temperature in the range of from 20 to 30° C. with Cu-Kalpha radiation having a wavelength of 0.15406 nm.

In another embodiment, the present invention relates to a composition comprising the crystalline form of Compound A of the present invention, wherein said composition is characterized by having an XRPD comprising a reflection at 2-theta angles in the range of from 4.4° to 4.6°, in the range of from 6.6° to 6.8°, and in the range of from 9.3° to 9.6°, when measured at a temperature in the range of from 20 to 30° C. with Cu-Kalpha radiation having a wavelength of 0.15406 nm.

In a further embodiment, the present invention relates to a composition comprising the crystalline form of Compound A of the present invention, wherein said composition is characterized by having an XRPD comprising a reflection at 2-theta angles in the range of from 4.4° to 4.5°, in the range of from 6.6° to 6.7°, and in the range of from 9.3° to 9.6°, when measured at a temperature in the range of from 20 to 30° C. with Cu-Kalpha radiation having a wavelength of 0.15406 nm.

In still a further embodiment, the present invention relates to a composition comprising the crystalline form of Compound A of the present invention, wherein said composition is characterized by having an XRPD comprising a reflection at 2-theta angles in the range of from 4.1° to 4.2°, in the range of from 6.6° to 6.7°, and in the range of from 9.4° to 9.6°, when measured at a temperature in the range of from 20 to 30° C. with Cu-Kalpha radiation having a wavelength of 0.15406 nm.

In a further embodiment, the present invention relates to a composition comprising the crystalline form of Compound A of the present invention, wherein said composition is characterized by having an XRPD comprising a reflection at 2-theta angles in the range of from 4.4° to 4.5°, in the range of from 6.6° to 6.7°, and in the range of from 9.3° to 9.5°, when measured at a temperature in the range of from 20 to 30° C. with Cu-Kalpha radiation having a wavelength of 0.15406 nm.

In yet another embodiment, the present invention relates to a composition comprising the crystalline form of Compound A of the present invention, wherein said composition is characterized by having an XRPD comprising a reflection at 2-theta angles of (4.4±0.2°) or (4.5±0.2°), a reflection at 2-theta angles of (6.6±0.2°) or (6.7±0.2°), and a reflection at 2-theta angles of (9.3±0.2°), (9.4±0.2°) or (9.5±0.2°), when measured at a temperature in the range of from 20 to 30° C. with Cu-Kalpha radiation having a wavelength of 0.15406 nm.

In still another embodiment, the present invention relates to a composition comprising the crystalline form of Compound A of the present invention, wherein said composition is characterized by having an XRPD comprising reflections at 2-theta angles of (4.4±0.2°), (6.6±0.2°) and (9.3±0.2°), when measured at a temperature in the range of from 20 to 30° C. with Cu-Kalpha radiation having a wavelength of 0.15406 nm.

In still another embodiment, the present invention relates to a composition comprising the crystalline form of Compound A of the present invention, wherein said composition is characterized by having an XRPD comprising reflections at 2-theta angles of (4.1±0.2°), (6.6±0.2°) and (9.4±0.2°), when measured at a temperature in the range of from 20 to 30° C. with Cu-Kalpha radiation having a wavelength of 0.15406 nm.

### Process for the Preparation of the Crystalline Form of Compound a of the Invention or of Compositions of Compound a Comprising the Same

In another aspect, the present invention relates to a process for the preparation of the crystalline form of Compound A of the present invention, or the composition of Compound A comprising the crystalline form of Compound A of the present invention, as defined in any one of the above-described aspects and corresponding embodiments, said process comprising:
(i) providing a solution of Compound A in a solvent mixture comprising water and at least one water-miscible organic solvent;
(ii) optionally, seeding the solution provided in (i) with the crystalline form of Compound A of the present invention, or with the composition comprising the crystalline form of Compound A of the present invention;
(iii) crystallizing Compound A of the present invention from the solution provided in (i), or optionally from the mixture obtained in (ii);
(iv) separating at least a part of the crystals or composition obtained in (iii) from the mother liquor;
(v) optionally, washing the crystals or composition obtained in (iv); and
(vi) drying the crystals or composition obtained in any one of steps (iii), (iv) or optionally (v).

The Compound A starting material in step (i) of the above-described process may be obtained as disclosed in WO 2017/060431 A1.

In one embodiment, the water-miscible organic solvent employed in step (i) of the above-described process is selected from the group consisting of water-miscible alcohols, ketones, nitriles, and any mixtures thereof. In a specific embodiment, the alcohol is selected from the group consisting of ethanol, n-propanol, isopropanol, and any mixtures thereof. In a preferred embodiment, the alcohol is isopropanol. In another embodiment, the ketone is acetone and/or methylethyl ketone. In still another embodiment, the nitrile is acetonitrile. In a particular preferred embodiment, the water-miscible organic solvent is isopropanol.

In one embodiment, the solvent mixture employed in step (i) of the above-described process comprises water in the range of from about 20 to 90% (w/w), preferably of from about 25 to 60% (w/w), more preferably of from about 30 to 40% (w/w), based on the weight of the solvent mixture. Most preferably, the solvent mixture comprises about 35% (w/w) water, based on the weight of the solvent mixture. In a particularly preferred embodiment, the solvent mixture in step (i) of the above-described process comprises water in the range of from about 30 to 50% (w/w), more preferably of from about 35 to 45% (w/w), for example of from about 40 to 45% (w/w), e.g. of about 43% (w/w), based on the weight of the solvent mixture.

In another embodiment, the solvent mixture employed in step (i) of the above-described process comprises at least one water-miscible organic solvent in the range of from about 10 to 80% (w/w), preferably of from about 40 to 75% (w/w), more preferably of from about 60 to 70% (w/w), based on the weight of the solvent mixture. Most preferably, the solvent mixture comprises about 65% (w/w) water-miscible organic solvent, based on the weight of the solvent mixture. In a particularly preferred embodiment, the solvent mixture in step (i) of the above-described process comprises at least one water-miscible organic solvent in the range of from about 50 to 70% (w/w), more preferably of from about 55 to 65% (w/w), for example of from about 55 to 60% (w/w), e.g. of about 57% (w/w), based on the weight of the solvent mixture.

In one embodiment, the concentration of Compound A employed per liter solvent mixture in step (i) of the above-described process is in the range of from about 80 to 250 g/L, preferably of from about 90 to 200 g/L, more preferably of from about 100 to 150 g/L and most preferably of from about 110 to 120 g/L, such as about 115 g/L. In a particularly preferred embodiment, the concentration of Compound A employed per liter solvent mixture in step (i) of the above-described process is in the range of from about 110 to 130 g/L, more preferably of from about 115 to 125 g/L, such as about 120 g/L.

The solution in step (i) may be prepared by first mixing Compound A with the water-miscible organic solvent and then adding water or vice-versa. The solution in step (i) of the above-described process may be prepared at room temperature or at elevated temperature, e.g. at a temperature in the range of from about 40° C. to about reflux temperature of the solvent or solvent mixture employed. In a particularly preferred embodiment, the solution in step (i) of the above-described process may be prepared at a temperature in the range of from about 70 to 90° C., more preferably of from about 75 to 85° C., such as about 80° C. Preferably, Compound A is first dissolved in the water-miscible organic solvent at an elevated temperature followed by the addition of water. The obtained solution may optionally be filtered in order to remove any potentially undissolved particles and/or foreign particles from the solution.

Seed crystals may optionally be employed to initiate crystallization and/or to control crystal growth. The amount of seed crystals employed in the optional seeding step (ii) of the above-described process is in the range of from about 1 to 20% (w/w), preferably of from about 2 to 10% (w/w) and most preferably of from about 3 to 5% (w/w), based on the amount of Compound A employed in step (i). In a particular preferred embodiment, about 1% (w/w) of seed crystals are employed in the optional seeding step (ii) of the above-described process. The seed crystals or composition may be prepared according to, or analogous to the procedures disclosed in Examples 2-1, 2-2 or 4 hereinafter.

Crystallization of Compound A in step (iii) of the above-described process usually appears spontaneously within a certain period of time and may depend on various factors such as type of water-miscible organic solvent, organic solvent/water ratio, crystallization temperature, Compound A concentration, mechanical agitation, vessel geometry etc. Crystallization may also be initiated by addition of an antisolvent, e.g. by adding additional water to the solution and/or by decreasing the temperature of the solution.

Once, the crystalline form of Compound A of the present invention or the composition comprising the crystalline form of Compound A of the present invention is obtained in sufficient amount, at least a part of the crystals or composition, preferably most of the crystals or composition, most preferably substantially all obtained crystals or composition are separated from the mother liquor in step (iv) of the above-described process. Thereby, the crystals or composition may be separated from the mother liquor by any conventional method known to the skilled person. In one embodiment, the crystals or composition are separated from the mother liquor by filtration, centrifugation, solvent evaporation and/or decantation. In a preferred embodiment, the crystals or composition are separated from the mother liquor by filtration and/or centrifugation. In a most preferred embodiment, the crystals or composition are separated from the mother liquor by filtration.

The isolated crystals or composition may be washed with a suitable solvent or solvent mixture in an optional step (v) of the above-described process. In a preferred embodiment, the crystals or composition are washed with water. In an alternative embodiment, the crystals or composition are washed with a mixture of water and a water-miscible organic solvent. In one embodiment, the water-miscible organic solvent is selected from the group consisting of alcohols, ketones, nitriles, and any mixtures thereof. In a specific embodiment, the alcohol is selected from the group consisting of methanol, ethanol, n-propanol, isopropanol, and any mixtures thereof. In a preferred embodiment, the alcohol is methanol or isopropanol. In another specific embodiment, the ketone is acetone and/or methyl ethyl ketone. In still another specific embodiment, the nitrile is acetonitrile. In a particular preferred embodiment, the water-miscible organic solvent is methanol or isopropanol. In one embodiment, the solvent mixture applied in optional step (v) of the above-described process comprises water in the range of from about 20 to 90% (w/w), preferably of from about 25 to 60% (w/w), more preferably of from about 30 to 40% (w/w), such as about 35% (w/w), based on the weight of the solvent mixture. In another embodiment, the solvent mixture employed in optional step (v) of the above-described process comprises at least one water-miscible organic solvent in the range of from about 10 to 80% (w/w), preferably of from about 40 to 75% (w/w), more preferably of from about 60 to 70% (w/w), based on the weight of the solvent mixture. Most preferably, the solvent mixture comprises about 65% (w/w) water-miscible organic solvent, based on the weight of the solvent mixture.

The obtained crystals or composition are finally dried in step (vi) of the above-described process. In one embodiment, the drying may be performed at a temperature in the range of from about room temperature to 100° C., preferably of from about 40 to 90° C., more preferably of from about 70 to 90° C., such as at about 80° C. In another embodiment, the drying may be performed at ambient pressure and/or under reduced pressure. In a preferred embodiment, the drying is performed under reduced pressure. For example, the drying is performed at a pressure of about 900 mbar or less, more preferably of about 100 mbar or less and most preferably of about 50 mbar or less, such as about 20 mbar or less. In still another embodiment, the drying may be performed for a period in the range of from about 1 to 72 hours, preferably of from about 6 to 48 hours. In one embodiment, drying may be performed under an inert gas stream, e.g. under a nitrogen stream.

In a specific embodiment, the present invention relates to a process for the preparation of the crystalline form of Compound A of the present invention, or the composition comprising the crystalline form of Compound A of the present invention, as defined in any one of the above-described aspects and corresponding embodiments, the process comprising:
(i) providing a solution of Compound A in a solvent mixture comprising about 35% (w/w) water and about 65% (w/w) isopropanol based on the weight of the solvent mixture, wherein Compound A is employed at a concentration of about 115 g/L solvent mixture;
(ii) optionally, seeding the solution provided in (i) with the crystalline form of Compound A of the present invention, or with the composition comprising the crystalline form of Compound A of the present invention;
(iii) crystallizing Compound A of the present invention from the solution provided in (i), or optionally from the mixture obtained in (ii);
(iv) separating at least a part of the crystals or composition obtained in (iii) from the mother liquor;
(v) optionally, washing the crystals or composition obtained in (iv); and
(vi) drying the crystals or composition obtained in any one of steps (iii), (iv) or optionally (v).

In another specific embodiment, the present invention relates to a process for the preparation of the crystalline form of Compound A of the present invention, or the composition comprising the crystalline form of Compound A of the present invention, as defined in any one of the above-described aspects and corresponding embodiments, the process comprising:
(i) providing a solution of Compound A in a solvent mixture comprising about 43% (w/w) water and about 57% (w/w) isopropanol based on the weight of the solvent mixture, wherein Compound A is employed at a concentration of about 120 g/L solvent mixture;
(ii) optionally, seeding the solution provided in (i) with the crystalline form of Compound A of the present invention, or with the composition comprising the crystalline form of Compound A of the present invention;
(iii) crystallizing Compound A of the present invention from the solution provided in (i), or optionally from the mixture obtained in (ii);
(iv) separating at least a part of the crystals or composition obtained in (iii) from the mother liquor;
(v) optionally, washing the crystals or composition obtained in (iv); and
(vi) drying the crystals or composition obtained in any one of steps (iii), (iv) or optionally (v).

In another aspect, the present invention relates to a process for the preparation of the crystalline form of Compound A of the present invention, or the composition of Compound A comprising the crystalline form of Compound A of the present invention, as defined in any one of the above-described aspects and corresponding embodiments, said process comprising:
(i) preparing a solution of Compound A in a water-miscible organic solvent, wherein the water-miscible organic solvent is not methanol;
(ii) crystallizing Compound A from the solution prepared in (i), comprising combining the solution prepared in (i) with water;
(iii) separating at least a part of the crystals or composition obtained in (ii) from the mother liquor;
(iv) optionally, washing the crystals or composition obtained in (iii); and
(v) drying the crystals or composition obtained in (ii) or (iii).

The Compound A starting material in step (i) of the above-described process may be obtained as disclosed in WO 2017/060431 A1.

In one embodiment, the water-miscible organic solvent employed in step (i) of the above-described process is selected from the group consisting of water-miscible alcohols, ketones, nitriles, and any mixtures thereof. In a specific embodiment, the alcohol is selected from the group consisting of ethanol, n-propanol, isopropanol, and any mixtures thereof. In a preferred embodiment, the alcohol is isopropanol. In another embodiment, the ketone is acetone and/or methylethyl ketone. In still another embodiment, the nitrile is acetonitrile. In a particular preferred embodiment, the water-miscible organic solvent is isopropanol.

In one embodiment, the concentration of Compound A employed per liter solvent mixture in step (i) of the above-described process is in the range of from about 300 to 400 g/L, preferably of from about 320 to 370 g/L, for example about 360 g/L.

The solution in step (i) of the above-described process may be prepared at room temperature or at elevated temperature, e.g. at a temperature in the range of from about 40° C. to about reflux temperature of the employed solvent. The obtained solution may optionally be filtered in order to remove any potentially undissolved particles and/or foreign particles from the solution.

Crystallization of Compound A in step (ii) of the above-described process is initiated by combining the solution provided in (i) with water, wherein water may be added to the solution obtained in (i) or vice versa, preferably water is added to the solution obtained in (i). Thereby, the solution provided in (i) may be combined with water at room temperature or at elevated temperature, e.g. at a temperature in the range of from about 40° C. to about reflux temperature. In one embodiment the final organic solvent/water ratio may be in the range of from about 1:0.5 to 1:5 (v:v), preferably from about 1:0.5 to 1:3 (v:v). Crystallization usually appears spontaneously within a certain period of time in step (ii) and may depend on various factors such as type of water-miscible organic solvent, organic solvent/water ratio, crystallization temperature, Compound A concentration, mechanical agitation, vessel geometry etc. Crystallization may also be initiated by adding additional water to the mixture and/or by decreasing the temperature of the mixture.

Once, the crystalline form of Compound A of the present invention or the composition comprising the crystalline form of Compound A of the present invention is obtained in sufficient amount, at least a part of the crystals or composition, preferably most of the crystals or composition, most preferably substantially all obtained crystals or composition are separated from the mother liquor in step (iv) of the above-described process. Thereby, the crystals or composition may be separated from the mother liquor by any conventional method known to the skilled person. In one embodiment, the crystals or composition are separated from the mother liquor by filtration, centrifugation, solvent evaporation and/or decantation. In a preferred embodiment, the crystals or composition are separated from the mother liquor by filtration and/or centrifugation. In a most preferred embodiment, the crystals or composition are separated from the mother liquor by filtration.

The isolated crystals or composition may be washed with a suitable solvent or solvent mixture in an optional step (v) of the above-described process. In a preferred embodiment, the crystals or composition are washed with water. In an alternative embodiment, the crystals are washed with a mixture of water and a water-miscible organic solvent. In one embodiment, the water-miscible organic solvent is selected from the group consisting of alcohols, ketones, nitriles, and any mixtures thereof. In a specific embodiment, the alcohol is selected from the group consisting of methanol, ethanol, n-propanol, isopropanol, and any mixtures thereof. In a preferred embodiment, the alcohol is isopropanol. In another specific embodiment, the ketone is acetone and/or methyl ethyl ketone. In still another specific embodiment, the nitrile is acetonitrile. In a particular preferred embodiment, the water-miscible organic solvent is methanol or isopropanol. In one embodiment, the solvent mixture applied in optional step (v) of the above-described process comprises water in the range of from about 20 to 90% (w/w), preferably of from about 25 to 60% (w/w), more preferably of from about 30 to 40% (w/w), such as about 35% (w/w), based on the weight of the solvent mixture. In another embodiment, the solvent mixture employed in optional step (v) of the above-described process comprises at least one water-miscible organic solvent in the range of from about 10 to 80% (w/w), preferably of from about 40 to 75% (w/w), more preferably of from about 60 to 70% (w/w), based on the weight of the solvent mixture. Most preferably, the solvent mixture comprises about 65% (w/w) water-miscible organic solvent, based on the weight of the solvent mixture.

The obtained crystals or composition are finally dried in step (vi) of the above-described process. In one embodiment, the drying may be performed at a temperature in the range of from about room temperature to 100° C., preferably of from about 40 to 90° C., more preferably of from about 70 to 90° C., such as at about 80° C. In another embodiment, the drying may be performed at ambient pressure and/or under reduced pressure. In a preferred embodiment, the drying is performed under reduced pressure. For example, the drying is performed at a pressure of about 900 mbar or less, more preferably of about 100 mbar or less and most preferably of about 50 mbar or less, such as about 20 mbar or less. In still another embodiment, the drying may be performed for a period in the range of from about 1 to 72 hours, preferably of from about 6 to 48 hours. In one embodiment, drying may be performed under an inert gas stream, e.g. under a nitrogen stream.

In another aspect, the present invention relates to a crystalline form of Compound A or a composition comprising a crystalline form of Compound A prepared, obtainable, or obtained by a process comprising:
(i) providing a solution of Compound A in a solvent mixture comprising water and at least one water-miscible organic solvent;
(ii) optionally, seeding the solution provided in (i) with the crystalline form of Compound A of the present invention, or with the composition comprising the crystalline form of Compound A of the present invention;
(iii) crystallizing Compound A of the present invention from the solution provided in (i), or optionally from the mixture obtained in (ii);
(iv) separating at least a part of the crystals or composition obtained in (iii) from the mother liquor;
(v) optionally, washing the crystals or composition obtained in (iv); and
(vi) drying the crystals or composition obtained in any one of steps (iii), (iv) or optionally (v).

In still another aspect, the present invention relates to a crystalline form of Compound A or a composition comprising a crystalline form of Compound A prepared, obtainable, or obtained by a process comprising:
(i) providing a solution of Compound A in a solvent mixture comprising about 35% (w/w) water and about 65% (w/w) isopropanol, based on the weight of the solvent mixture, wherein Compound A is employed at a concentration of about 115 g/L solvent mixture;
(ii) optionally, seeding the solution provided in (i) with the crystalline form of Compound A of the present invention, or with the composition comprising the crystalline form of Compound A of the present invention;
(iii) crystallizing Compound A of the present invention from the solution provided in (i), or optionally from the mixture obtained in (ii);
(iv) separating at least a part of the crystals or composition obtained in (iii) from the mother liquor;
(v) optionally, washing the crystals or composition obtained in (iv); and
(vi) drying the crystals or composition obtained in any one of steps (iii), (iv) or optionally (v).

In yet another aspect, the present invention relates to a crystalline form of Compound A or a composition comprising a crystalline form of Compound A prepared, obtainable, or obtained by a process comprising:
(i) providing a solution of Compound A in a solvent mixture comprising about 43% (w/w) water and about 57% (w/w) isopropanol, based on the weight of the solvent mixture, wherein Compound A is employed at a concentration of about 120 g/L solvent mixture;
(ii) optionally, seeding the solution provided in (i) with the crystalline form of Compound A of the present invention, or with the composition comprising the crystalline form of Compound A of the present invention;
(iii) crystallizing Compound A of the present invention from the solution provided in (i), or optionally from the mixture obtained in (ii);
(iv) separating at least a part of the crystals or composition obtained in (iii) from the mother liquor;
(v) optionally, washing the crystals or composition obtained in (iv); and
(vi) drying the crystals or composition obtained in any one of steps (iii), (iv) or optionally (v).

In yet another aspect, the present invention relates to a crystalline form of Compound A or a composition comprising a crystalline form of Compound A prepared, obtainable, or obtained by a process comprising:
(i) preparing a solution of Compound A in a water-miscible organic solvent, wherein the water-miscible organic solvent is not methanol;
(ii) crystallizing Compound A from the solution prepared in (i), comprising combining the solution prepared in (i) with water;
(iii) separating at least a part of the crystals or composition obtained in (ii) from the mother liquor;
(iv) optionally, washing the crystals or composition obtained in (iii); and
(v) drying the crystals or composition obtained in (ii) or (iii).

In another aspect, the present invention relates to a crystalline form of Compound A or a composition comprising a crystalline form of Compound A prepared, obtainable, or obtained by any process described herein.

### Pharmaceutical Compositions and Process for the Preparation Thereof

In a further aspect, the present invention relates to the use of Compound A or a pharmaceutically acceptable salt thereof, the crystalline form of Compound A of the present invention, or the composition of Compound A comprising the crystalline form of Compound A of the present invention, all as defined in the above-described aspects and corresponding embodiments, for the preparation of a pharmaceutical composition.

In a particular aspect, the present invention relates to a pharmaceutical composition comprising Compound A or a pharmaceutically acceptable salt thereof, the crystalline form of Compound A of the present invention, or the composition comprising the crystalline form of Compound A of the present invention, and at least one pharmaceutically acceptable excipient.

The pharmaceutical composition of the present invention may be characterized by any one of the following embodiments or by combining two or more of the following embodiments.

In one embodiment, the at least one pharmaceutically acceptable excipient comprised in the pharmaceutically composition of the present invention is selected from the group consisting of fillers (diluents), binders, surfactants, disintegrants, lubricants, and any combinations thereof.

In another embodiment, the at least one pharmaceutically acceptable excipient comprised in the pharmaceutical composition of the present invention comprises at least one surfactant. In a particular embodiment, the at least one surfactant comprises an anionic surfactant. In a specific embodiment, the at least one surfactant is selected from the group consisting of sodium lauryl sulfate, ammonium lauryl sulfate, sodium lauryl ether sulfate, sodium myristyl ether sulfate, sodium stearate, sodium lauroyl sarcosinate, and any combination thereof. In a preferred embodiment the at least one surfactant comprises or is sodium lauryl sulfate. In one embodiment, the at least one surfactant is present in the pharmaceutical composition in an amount of from about 0.1 to 5% (w/w), preferably of from about 0.2 to 2% (w/w), more preferably of from about 0.5 to 1% (w/w), based on the weight of the pharmaceutical composition. Most preferably, the at least one surfactant is present in the pharmaceutical composition in an amount of about 0.5% (w/w), based on the weight of the pharmaceutical composition. For example, the at least one surfactant is sodium lauryl sulfate present in the pharmaceutical composition in an amount of about 0.5% (w/w), based on the weight of the pharmaceutical composition.

In a further embodiment, the at least one pharmaceutically acceptable excipient comprised in the pharmaceutical composition of the present invention comprises at least one surfactant, and at least one binder. In one embodiment, the at least one binder is selected from the group consisting of starch, hypromellose, polyvinylpyrrolidone, sodium carboxymethyl cellulose, hydroxypropyl cellulose, methyl cellulose, ethyl cellulose, gelatin, sucrose, and any combination thereof. Preferably, the at least one binder comprises or is hypromellose. In a particular embodiment, the at least one binder is present in the pharmaceutical composition in an amount of from about 1 to 5% (w/w), preferably of from about 2 to 3% (w/w), based on the weight of the pharmaceutical composition. For example, the at least one binder is hypromellose present in the pharmaceutical composition in an amount of about 3% (w/w), based on the weight of the pharmaceutical composition.

In a specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate, and hypromellose.

In still another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate in an amount of from about 0.1 to 5% (w/w), preferably of from about 0.2 to 2% (w/w), more preferably of from about 0.5 to 1% (w/w), such as of about 0.5% (w/w), and hypromellose in an amount of from about 1 to 5% (w/w), preferably of from about 2 to 3% (w/w), such as of about 3% (w/w), based on the weight of the pharmaceutical composition.

In yet another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, 0.5% (w/w) sodium lauryl sulfate, and 3% (w/w) hypromellose, based on the weight of the pharmaceutical composition.

In one embodiment, the at least one pharmaceutically acceptable excipient comprised in the pharmaceutical composition of the present invention comprises at least one surfactant, at least one binder, and at least one filler. In a further embodiment, the at least one filler is selected from the group consisting of starch (e.g. potato starch, corn starch, Starch 1500®), pre-gelatinized starch, sorbitol, mannitol, xylitol, sucrose, dextrose, isomalt, calcium carbonate, magnesium carbonate, dibasic calcium phosphate, tribasic calcium phosphate, trehalose, lactose, glucose, maltodextrin, cyclodextrin, cellulose, microcrystalline cellulose, silicified microcrystalline cellulose, and any combination thereof. Preferably, the at least one filler comprises microcrystalline cellulose and/or mannitol, most preferably the at least one filler comprises or is a combination of microcrystalline cellulose and mannitol. In a particular embodiment, the at least one filler is present in the pharmaceutical composition in an amount of from about 70 to 90% (w/w), preferably of from about 75 to 85% (w/w) and most preferably of about 80% (w/w), based on the weight of the pharmaceutical composition.

In a specific embodiment, the at least one filler comprises a first filler and a second filler, wherein the total amount of the first filler and the second filler present in the pharmaceutical composition is in the range of from about 70 to 90% (w/w), preferably of from about 75 to 85% (w/w) and most preferably the total amount of the first filler and the second filler present in the pharmaceutical composition is about 80% (w/w), based on the weight of the pharmaceutical composition. In a specific embodiment, the first filler comprises microcrystalline cellulose and the second filler comprises mannitol. In a preferred embodiment the first filler is microcrystalline cellulose and the second filler is mannitol.

In another embodiment, the first filler is mannitol present in the pharmaceutical composition in an amount of about 60% (w/w) e.g. of about 59% (w/w) and the second filler is microcrystalline cellulose present in the pharmaceutical composition in an amount of about 20% (w/w) e.g. of about 21% (w/w), based on the weight of the pharmaceutical composition.

In still another embodiment, the at least one filler comprises a first filler and a second filler, wherein the ratio by weight of the first filler to the second filler is in the range of from about 2.5:1 to 5:1 (e.g. 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1). In one embodiment, the at least one filler comprises a first filler and a second filler, wherein the ratio by weight of the first filler to the second filler is in the range of from about 2.9:1 to 3.6:1 (e.g. 2.9:1, 3:1, 3.1:1, 3.2:1, 3.3:1, 3.4:1, 3.5:1, 3.6:1). In a specific embodiment the at least one filler comprises a first filler and a second filler, wherein the ratio by weight of the first filler to the second filler is about 2.9:1 or 3.6:1. In a specific embodiment, the first filler comprises mannitol and the second filler comprises microcrystalline cellulose. In a preferred embodiment the first filler is mannitol and the second filler is microcrystalline cellulose.

In a specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate, hypromellose, mannitol, and microcrystalline cellulose.

In still another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate in an amount of from about 0.2 to 2% (w/w), preferably of from about 0.5 to 1% (w/w), such as of about 0.5% (w/w), hypromellose in an amount of from about 1 to 5% (w/w), preferably of from about 2 to 3% (w/w), such as of about 3% (w/w), microcrystalline cellulose and mannitol, wherein the total amount of microcrystalline cellulose and mannitol is in the range of from about 70 to 90% (w/w), preferably of from about 75 to 85% (w/w) and most preferably the total amount of microcrystalline cellulose and mannitol in the pharmaceutical composition is about 80% (w/w), based on the weight of the pharmaceutical composition.

In another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate in an amount of from about 0.2 to 2% (w/w), preferably of from about 0.5 to 1% (w/w), such as of about 0.5% (w/w), hypromellose in an amount of from about 1 to 5% (w/w), preferably of from about 2 to 3% (w/w), such as of about 3% (w/w), microcrystalline cellulose in an amount of about 20% (w/w) e.g. of about 21% (w/w) and mannitol in an amount of about 60% (w/w) e.g. of about 59% (w/w), based on the weight of the pharmaceutical composition.

In yet another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, 0.5% (w/w) sodium lauryl sulfate, 3% (w/w) hypromellose, 21% (w/w) microcrystalline cellulose, and 59% (w/w) mannitol, based on the weight of the pharmaceutical composition.

In another embodiment, the at least one pharmaceutically acceptable excipient comprised in the pharmaceutical composition of the present invention comprises at least one surfactant, at least one binder, at least one filler and at least one disintegrant. In one embodiment the at least one disintegrant is selected from the group consisting of sodium carboxymethyl starch, low-substituted hydroxypropyl cellulose, crospovidone, croscarmellose sodium, croscarmellose, methyl cellulose, pregelatinized starch, sodium alginate, and any combination thereof. Preferably, the at least one disintegrant comprises or is crospovidone. In a particular embodiment, the at least one disintegrant is present in the pharmaceutical composition in an amount of from about 1 to 5% (w/w), preferably of from about 3 to 4% (w/w), such as of about 3% (w/w), based on the weight of the pharmaceutical composition. For example, the at least one disintegrant is crospovidone present in the pharmaceutical composition in an amount of about 3% (w/w), based on the weight of the pharmaceutical composition.

In a specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate, hypromellose, mannitol, microcrystalline cellulose, and crospovidone.

In still another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate in an amount of from about 0.2 to 2% (w/w), preferably of from about 0.5 to 1% (w/w), such as of about 0.5% (w/w), hypromellose in an amount of from about 1 to 5% (w/w), preferably of from about 2 to 3% (w/w), such as of about 3% (w/w), microcrystalline cellulose and mannitol, wherein the total amount of microcrystalline cellulose and mannitol is in the range of from about 70 to 90% (w/w), preferably of from about 75 to 85% (w/w) and most preferably the total amount microcrystalline cellulose and mannitol in the pharmaceutical composition is about 80% (w/w), and crospovidone in an amount of from about 1 to 5% (w/w), preferably of from about 3 to 4% (w/w), such as of about 3% (w/w), based on the weight of the pharmaceutical composition.

In still another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate in an amount of from about 0.2 to 2% (w/w), preferably of from about 0.5 to 1% (w/w), such as of about 0.5% (w/w), hypromellose in an amount of from about 1 to 5% (w/w), preferably of from about 2 to 3% (w/w), such as of about 3% (w/w), microcrystalline cellulose in an amount of about 20% (w/w) e.g. of about 21% (w/w), mannitol in an amount of about 60% (w/w) e.g. of about 59% (w/w) and crospovidone in an amount of from about 1 to 5% (w/w), preferably of from about 3 to 4% (w/w), such as of about 3% (w/w), based on the weight of the pharmaceutical composition.

In yet another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, 0.5% (w/w) sodium lauryl sulfate, 3% (w/w) hypromellose, 21% (w/w) microcrystalline cellulose, 59% (w/w) mannitol, and 3% (w/w) crospovidone, based on the weight of the pharmaceutical composition.

In still another embodiment, the at least one pharmaceutically acceptable excipient comprised in the pharmaceutical composition of the present invention comprises at least one surfactant, at least one binder, at least one filler, at least one disintegrant, and at least one lubricant. In one embodiment, the at least one lubricant is selected from the group consisting of zinc stearate, glyceryl monostearate, glyceryl palmitate stearate, magnesium stearate, sodium fumarate and any combination thereof. Preferably, the at least one lubricant comprises or is magnesium stearate. In a particular embodiment, the at least one lubricant is present in the pharmaceutical composition in an amount of from about 0.5 to 2% (w/w), preferably in an amount of about 1% (w/w), based on the weight of the pharmaceutical composition. For example, the at least one lubricant is magnesium stearate present in the pharmaceutical composition in an amount of about 1% (w/w), based on the weight of the pharmaceutical composition.

In a specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate, hypromellose, mannitol, microcrystalline cellulose, crospovidone, and magnesium stearate.

In still another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate in an amount of from about 0.2 to 2% (w/w), preferably of from about 0.5 to 1% (w/w), such as of about 0.5% (w/w), hypromellose in an amount of from about 1 to 5% (w/w), preferably of from about 2 to 3% (w/w), such as of about 3% (w/w), microcrystalline cellulose and mannitol, wherein the total amount of microcrystalline cellulose and mannitol is in the range of from about 70 to 90% (w/w), preferably of from about 75 to 85% (w/w) and most preferably the total amount microcrystalline cellulose and mannitol in the pharmaceutical composition is about 80% (w/w), crospovidone in an amount of from about 1 to 5% (w/w), preferably of from about 3 to 4% (w/w), such as of about 3% (w/w), and magnesium stearate in an amount of from about 0.5 to 2% (w/w), preferably in an amount of about 1% (w/w), based on the weight of the pharmaceutical composition.

In still another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, sodium lauryl sulfate in an amount of from about 0.2 to 2% (w/w), preferably of from about 0.5 to 1% (w/w), such as of about 0.5% (w/w), hypromellose in an amount of from about 1 to 5% (w/w), preferably of from about 2 to 3% (w/w), such as of about 3% (w/w), microcrystalline cellulose in an amount of about 20% (w/w) e.g. of about 21% (w/w), mannitol in an amount of about 60% (w/w) e.g. of about 59% (w/w), crospovidone in an amount of from about 1 to 5% (w/w), preferably of from about 3 to 4% (w/w), such as of about 3% (w/w), and magnesium stearate in an amount of from about 0.5 to 2% (w/w), preferably in an amount of about 1% (w/w), based on the weight of the pharmaceutical composition.

In yet another specific embodiment, the present invention relates to a pharmaceutical composition comprising Compound A or a pharmaceutically acceptable salt thereof, the crystalline form of Compound A of the present invention, or the composition comprising the crystalline form of Compound A of the present invention, 0.5% (w/w) sodium lauryl sulfate, 3% (w/w) hypromellose, 21% (w/w) microcrystalline cellulose, 59% (w/w) mannitol, 3% (w/w) crospovidone, and 1% (w/w) magnesium stearate, based on the weight of the pharmaceutical composition.

In a further embodiment, the present invention relates to a pharmaceutical composition comprising a predetermined and/or effective amount of Compound A or a pharmaceutically acceptable salt thereof, the crystalline form of Compound A of the present invention, or the composition comprising the crystalline form of Compound A of the present invention. In one embodiment, the predetermined and/or effective amount may be in the range of from about 1 to 200 mg, preferably of from about 5 to 100 mg, and most preferably of from about 10 to 60 mg, such as from about 10 to 45 mg, calculated as free Compound A. For example, the predetermined and/or effective amount is selected from the group consisting of 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg and 200 mg, calculated as free Compound A. In a preferred embodiment, the predetermined and/or effective amount is selected from the group consisting of 1 mg, 5 mg, 10 mg, 30 mg, 45 mg, 50 mg and 60 mg, calculated as free Compound A. In a particularly preferred embodiment, the predetermined and/or effective amount is selected from the group consisting of 10 mg, 30 mg and 45 mg, calculated as free Compound A. Most preferably, the predetermined and/or effective amount is 30 mg or 45 mg, calculated as free Compound A.

In another embodiment, the present invention relates to a pharmaceutical composition comprising an amount of Compound A or a pharmaceutically acceptable salt thereof, the crystalline form of Compound A of the present invention, or the composition comprising the crystalline form of Compound A of the present invention, sufficient to deliver between about 1 to 200 mg, preferably between about 5 to 100 mg and most preferably between about 10 to 60 mg, such as between about 10 to 45 mg of Compound A per unit dosage form (e.g. per tablet), calculated as free Compound A. In a specific embodiment, the invention relates to a pharmaceutical composition comprising an amount of Compound A or a pharmaceutically acceptable salt thereof, the crystalline form of Compound A of the present invention, or the composition comprising the crystalline form of Compound A of the present invention, sufficient to deliver about 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg or 200 mg of Compound A per unit dosage form (e.g. per tablet), calculated as free Compound A. In a preferred embodiment, the present invention relates to a pharmaceutical composition comprising an amount of Compound A or a pharmaceutically acceptable salt thereof, the crystalline form of Compound A of the present invention, or the composition comprising the crystalline form of Compound A of the present invention, sufficient to deliver about 1 mg, 5 mg, 10 mg, 30 mg, 45 mg, 50 mg or 60 mg of Compound A per unit dosage form (e.g. per tablet), calculated as free Compound A. In a particularly preferred embodiment, the present invention relates to a pharmaceutical composition comprising an amount of Compound A or a pharmaceutically acceptable salt thereof, the crystalline form of Compound A of the present invention, or the composition comprising the crystalline form of Compound A of the present invention, sufficient to deliver about 10 mg, 30 mg or 45 mg of Compound A per unit dosage form (e.g. per tablet), calculated as free Compound A. Most preferably, the present invention relates to a pharmaceutical composition comprising an amount of Compound A or a pharmaceutically acceptable salt thereof, the crystalline form of Compound A of the present invention, or the composition comprising the crystalline form of Compound A of the present invention, sufficient to deliver about 30 mg or 45 mg of Compound A per unit dosage form (e.g. per tablet), calculated as free Compound A.

In an additional embodiment, the invention relates to a pharmaceutical composition comprising Compound A or a pharmaceutically acceptable salt thereof, the crystalline form of Compound A of the present invention, or the composition comprising the crystalline form of Compound A of the present invention, in an amount of from about 5 to 20% (w/w), preferably of from about 7.5 to 15% (w/w), such as of about 7.5, 12.5 or 15% (w/w), calculated as free Compound A and based on the weight of the pharmaceutical composition.

In a specific embodiment, the present invention relates to a pharmaceutical composition comprising, substantially comprising, or consisting of 12.5% (w/w) Compound A or a pharmaceutically acceptable salt thereof, the crystalline form of Compound A of the present invention, or the composition comprising the crystalline form of Compound A of the present invention, calculated as free Compound A, 0.5% (w/w) sodium lauryl sulfate, 3% (w/w) hypromellose, 21% (w/w) microcrystalline cellulose, 59% (w/w) mannitol, 3% (w/w) crospovidone, and 1% (w/w) magnesium stearate.

In another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from about 9.2° to 9.6°, when measured at a temperature in the range of from 20 to 30° C. with Cu-Kalpha radiation having a wavelength of 0.15406 nm.

In another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from about 9.3° to 9.6°, when measured at a temperature in the range of from 20 to 30° C. with Cu-Kalpha radiation having a wavelength of 0.15406 nm.

In a further embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from about 9.3° to 9.5°, when measured at a temperature in the range of from 20 to 30° C. with Cu-Kalpha radiation having a wavelength of 0.15406 nm.

In a particular embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles of (9.3±0.2°), (9.4±0.2°), or (9.5±0.2)° 2-theta, when measured at a temperature in the range of from 20 to 30° C. with Cu-Kalpha radiation having a wavelength of 0.15406 nm.

In yet another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from about 9.4° to 9.6°, when measured at a temperature in the range of from 20 to 30° C. with Cu-Kalpha radiation having a wavelength of 0.15406 nm.

In another particular embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from about (9.4±0.2°) or (9.5±0.2°) when measured at a temperature in the range of from 20 to 30° C. with Cu-Kalpha radiation having a wavelength of 0.15406 nm.

In yet another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.0° to 4.6°, when measured at a temperature in the range of from 20 to 30° C. with Cu-Kalpha radiation having a wavelength of 0.15406 nm.

In still another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.1° to 4.5°, when measured at a temperature in the range of from 20 to 30° C. with Cu-Kalpha radiation having a wavelength of 0.15406 nm.

In another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.4° to 4.6°, when measured at a temperature in the range of from 20 to 30° C. with Cu-Kalpha radiation having a wavelength of 0.15406 nm.

In still another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.4° to 4.5°, when measured at a temperature in the range of from 20 to 30° C. with Cu-Kalpha radiation having a wavelength of 0.15406 nm.

In another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.10 to 4.2°, when measured at a temperature in the range of from 20 to 30° C. with Cu-Kalpha radiation having a wavelength of 0.15406 nm.

In a particular embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising reflections at 2-theta angles of (4.1±0.2°), (4.2±0.2°), (4.4±0.2°), or (4.5±0.2°), when measured at a temperature in the range of from 20 to 30° C. with Cu-Kalpha radiation having a wavelength of 0.15406 nm.

In one embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.0° to 4.6° and in the range of from 9.2° to 9.6°, when measured at a temperature in the range of from 20 to 30° C. with Cu-Kalpha radiation having a wavelength of 0.15406 nm.

In another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.1° to 4.5° and in the range of from 9.3° to 9.6°, when measured at a temperature in the range of from 20 to 30° C. with Cu-Kalpha radiation having a wavelength of 0.15406 nm.

In another embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.4° to 4.6° and in the range of from 9.3° to 9.6°, when measured at a temperature in the range of from 20 to 30° C. with Cu-Kalpha radiation having a wavelength of 0.15406 nm.

In a further embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.4° to 4.5° and in the range of from 9.30 to 9.5°, when measured at a temperature in the range of from 20 to 30° C. with Cu-Kalpha radiation having a wavelength of 0.15406 nm.

In a further embodiment, the present invention relates to a pharmaceutical composition comprising the crystalline form of Compound A of the present invention, or the composition comprising the crystalline form of Compound A of the present invention and at least one pharmaceutically acceptable excipient, wherein said pharmaceutical composition is characterized by having an XRPD comprising a reflection at 2-theta angles in the range of from 4.1° to 4.2° and in the range of from 9.4° to 9.6°, when measured at a temperature in the range of from 20 to 30° C. with Cu-Kalpha radiation having a wavelength of 0.15406 nm.

In still another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles of (4.1±0.2°) and (9.4±0.2°), when measured at a temperature in the range of from 20 to 30° C. with Cu-Kalpha radiation having a wavelength of 0.15406 nm.

In still another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles of (4.2±0.2°) and (9.6±0.2°), when measured at a temperature in the range of from 20 to 30° C. with Cu-Kalpha radiation having a wavelength of 0.15406 nm.

In yet another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles of (4.4±0.2°) or (4.5±0.2°) and a reflection at 2-theta angles of (9.3±0.2°), (9.4±0.2°) or (9.5±0.2°), when measured at a temperature in the range of from 20 to 30° C. with Cu-Kalpha radiation having a wavelength of 0.15406 nm.

In another preferred embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising reflections at 2-theta angles of (4.4±0.2°) and (9.3±0.2°), when measured at a temperature in the range of from 20 to 30° C. with Cu-Kalpha radiation having a wavelength of 0.15406 nm.

In another preferred embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising reflections at 2-theta angles of (4.5±0.2°) and (9.4±0.2°), when measured at a temperature in the range of from 20 to 30° C. with Cu-Kalpha radiation having a wavelength of 0.15406 nm.

In another preferred embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising reflections at 2-theta angles of (4.5±0.2°) and (9.5±0.2°), when measured at a temperature in the range of from 20 to 30° C. with Cu-Kalpha radiation having a wavelength of 0.15406 nm.

In a further embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles in the range of from 6.5° to 6.8°, when measured at a temperature in the range of from 20 to 30° C. with Cu-Kalpha radiation having a wavelength of 0.15406 nm.

In another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles in the range of from 6.6° to 6.8°, when measured at a temperature in the range of from 20 to 30° C. with Cu-Kalpha radiation having a wavelength of 0.15406 nm.

In another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles in the range of from 6.6° to 6.7°, when measured at a temperature in the range of from 20 to 30° C. with Cu-Kalpha radiation having a wavelength of 0.15406 nm.

In a particular embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles of (6.5±0.2°), (6.6±0.2°), or (6.7±0.2°), when measured at a temperature in the range of from 20 to 30° C. with Cu-Kalpha radiation having a wavelength of 0.15406 nm.

In one embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles in the range of from 4.0° to 4.6°, in the range of from 6.5° to 6.8°, and in the range of from 9.2° to 9.6°, when measured at a temperature in the range of from 20 to 30° C. with Cu-Kalpha radiation having a wavelength of 0.15406 nm.

In a further embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles in the range of from 4.1° to 4.5°, in the range of from 6.6° to 6.7°, and in the range of from 9.3° to 9.6°, when measured at a temperature in the range of from 20 to 30° C. with Cu-Kalpha radiation having a wavelength of 0.15406 nm.

In another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles in the range of from 4.4° to 4.6°, in the range of from 6.6° to 6.8°, and in the range of from 9.3° to 9.6°, when measured at a temperature in the range of from 20 to 30° C. with Cu-Kalpha radiation having a wavelength of 0.15406 nm.

In a further embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles in the range of from 4.4° to 4.5°, in the range of from 6.6° to 6.7°, and in the range of from 9.3° to 9.6°, when measured at a temperature in the range of from 20 to 30° C. with Cu-Kalpha radiation having a wavelength of 0.15406 nm.

In a further embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles in the range of from 4.4° to 4.5°, in the range of from 6.6° to 6.7°, and in the range of from 9.3° to 9.5°, when measured at a temperature in the range of from 20 to 30° C. with Cu-Kalpha radiation having a wavelength of 0.15406 nm.

In still another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles in the range of from 4.1° to 4.2°, in the range of from 6.6° to 6.7°, and in the range of from 9.4° to 9.6°, when measured at a temperature in the range of from 20 to 30° C. with Cu-Kalpha radiation having a wavelength of 0.15406 nm.

In yet another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising a reflection at 2-theta angles of (4.4±0.2°) or (4.5±0.2°), a reflection at 2-theta angles of (6.6±0.2°) or (6.7±0.2°), and a reflection at 2-theta angles of (9.3±0.2°), (9.4±0.2°) or (9.5±0.2°), when measured at a temperature in the range of from 20 to 30° C. with Cu-Kalpha radiation having a wavelength of 0.15406 nm.

In still another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising reflections at 2-theta angles of (4.4±0.2°), (6.6±0.2°) and (9.3±0.2°), when measured at a temperature in the range of from 20 to 30° C. with Cu-Kalpha radiation having a wavelength of 0.15406 nm.

In still another embodiment, the present invention relates to a pharmaceutical composition comprising the crystalline form of Compound A of the present invention, or the composition comprising the crystalline form of Compound A of the present invention and at least one pharmaceutical acceptable excipient, wherein said pharmaceutical composition is characterized by having an XRPD comprising reflections at 2-theta angles of (4.1±0.2°), (6.6±0.2°) and (9.4±0.2°), when measured at a temperature in the range of from 20 to 30° C. with Cu-Kalpha radiation having a wavelength of 0.15406 nm.

In one embodiment, the above-mentioned reflections of the pharmaceutical composition are due to reflections of the crystalline form of Compound A of the present invention. In a particular embodiment, the above-mentioned reflections of the pharmaceutical composition are not due to reflections of pharmaceutically acceptable excipients present in the pharmaceutical composition.

In a further embodiment, the pharmaceutical composition of the present invention is an oral solid dosage form. In a particular embodiment, the pharmaceutical composition of the present invention is a capsule, preferably a hard gelatin capsule or a tablet, preferably a film-coated tablet.

In a specific embodiment, the pharmaceutical composition of the present invention is a film-coated tablet comprising a tablet core and a film-coating. In a particular embodiment, the film coating comprises at least one pharmaceutically acceptable excipient selected from the group consisting of film-forming agent, plasticizer, pigment, anti-adherent, and any combinations thereof. In a particular embodiment, the film coating comprises hypromellose, macrogol (e.g. macrogol 6000, macrogol 3000, macrogol 400, macrogol 4000, macrogol 8000), titanium dioxide, talcum and iron oxide. In one embodiment the film-coating is an Opadry film coating powder. In a particular embodiment, the film coating is free of titanium dioxide. In a particular embodiment, the film coating comprises calcium carbonate and/or talc.

In another aspect, the invention relates to a process for the preparation of a pharmaceutical composition comprising:
(i) mixing Compound A or a pharmaceutically acceptable salt thereof, the crystalline form of Compound A of the present invention, or the composition comprising the crystalline form of Compound A of the present invention, all as defined in the above-described aspects and corresponding embodiments, with at least one pharmaceutically acceptable excipient;
(ii) subjecting the mixture obtained in (i) to a wet granulation comprising contacting the mixture with a granulation liquid, wherein the granulation liquid comprises at least one surfactant and optionally at least one binder dissolved in the granulation liquid; (iii) drying the granules obtained in (ii);
(iv) optionally, mixing the granules obtained in (iii) with one or more additional pharmaceutically acceptable excipient(s);
(v) optionally, tableting the granules obtained in (iii) or the mixture obtained in (iv); and (vi) optionally, film-coating the tablets obtained in (v).

In one embodiment, the at least one pharmaceutically acceptable excipient employed in step (i) comprises at least one filler. In one embodiment, the at least one filler is selected from the group consisting of starch (e.g. potato starch, corn starch, Starch 1500®), pre-gelatinized starch, sorbitol, mannitol, xylitol, sucrose, dextrose, isomalt, calcium carbonate, magnesium carbonate, dibasic calcium phosphate, tribasic calcium phosphate, trehalose, lactose, glucose, maltodextrin, cyclodextrin, cellulose, microcrystalline cellulose, silicified microcrystalline cellulose, and any combination thereof. Preferably, the at least one filler comprises microcrystalline cellulose and/or mannitol, most preferably the at least one filler comprises or is a combination of microcrystalline cellulose and mannitol.

In another embodiment, the at least one pharmaceutically acceptable excipient employed in step (i) comprises at least one filler and at least one binder. In one embodiment, the at least one binder is selected from the group consisting of starch, hypromellose, polyvinylpyrrolidone, sodium carboxymethyl cellulose, hydroxypropyl cellulose, methyl cellulose, ethyl cellulose, gelatin, sucrose, and any combination thereof. Preferably, the at least one binder comprises or is hypromellose.

Optionally, the one or more pharmaceutically acceptable excipients employed in step (i) of the above defined process may be subjected to a size reduction and/or sieving step prior to mixing. Mixing may be accomplished by commonly used mixing methods, for example by using a free fall blender.

In another embodiment, the granulation liquid employed in step (ii) of the above defined process comprises or is water, ethanol or a mixture thereof. In a preferred embodiment, the granulation liquid is water, more precisely purified water. In a particular embodiment, the surfactant and the optional binder are at least partially dissolved, preferably completely dissolved in the granulation liquid. Wet granulation may be accomplished by commonly used wet granulation methods, for example by using a high shear granulator. Optionally, the obtained granules are subjected to one or more sieving step(s), wherein the sieving steps may be applied prior and/or after the drying step (iii).

In still another embodiment, the one or more additional pharmaceutically acceptable excipient employed in optional step (iv) comprises at least one filler, binder, disintegrant, lubricant and any combination thereof.

In one embodiment the at least one filler is selected from the group consisting of starch (e.g. potato starch, corn starch, Starch 1500©), pre-gelatinized starch, sorbitol, mannitol, xylitol, sucrose, dextrose, isomalt, calcium carbonate, magnesium carbonate, dibasic calcium phosphate, tribasic calcium phosphate, trehalose, lactose, glucose, maltodextrin, cyclodextrin, cellulose, microcrystalline cellulose, silicified microcrystalline cellulose, and any combination thereof. Preferably, the at least one filler comprises microcrystalline cellulose and/or mannitol, most preferably the at least one filler comprises or is a combination of microcrystalline cellulose and mannitol.

In another embodiment, the at least one binder is selected from the group consisting of starch, hypromellose, polyvinylpyrrolidone, sodium carboxymethyl cellulose, hydroxypropyl cellulose, methyl cellulose, ethyl cellulose, gelatin, sucrose, and any combination thereof. Preferably, the at least one binder comprises or is hypromellose.

In another embodiment, the at least one disintegrant is selected from the group consisting of sodium carboxymethyl starch, low-substituted hydroxypropyl cellulose, crospovidone, croscarmellose sodium, croscarmellose, methyl cellulose, pregelatinized starch, sodium alginate, and any combination thereof. Preferably, the at least one disintegrant comprises or is crospovidone.

In still another embodiment, the at least one lubricant is selected from the group consisting of zinc stearate, glyceryl monostearate, glyceryl palmitate stearate, magnesium stearate, sodium fumarate and any combination thereof. Preferably, the at least one lubricant comprises or is magnesium stearate.

Optionally, the one or more pharmaceutically acceptable excipient employed in optional step (iv) of the above defined process may be subjected to a size reduction and/or sieving step prior to mixing. Mixing may be accomplished by commonly used mixing methods, for example by using a free fall blender.

The optional tableting step (iv) of the above-defined process may be accomplished by commonly used tableting methods, for example by using a suitable tablet press.

The optional film-coating step (v) of the above-defined process may be accomplished by commonly used film-coating methods, for example by using a suitable pan coater.

In still another aspect, the present invention relates to a pharmaceutical composition prepared, obtainable, or obtained by a process comprising:
(i) mixing Compound A or a pharmaceutically acceptable salt thereof, the crystalline form of Compound A of the present invention, or the composition comprising the crystalline form of Compound A of the present invention, with at least one pharmaceutically acceptable excipient;
(ii) subjecting the mixture obtained in (i) to a wet granulation comprising contacting the mixture with a granulation liquid, wherein said granulation liquid comprises at least one surfactant and optionally at least one binder dissolved in the granulation liquid;
(iii) drying the granules obtained in (ii);
(iv) optionally, mixing the granules obtained in (iii) with one or more additional excipient(s);
(v) optionally, tableting the granules obtained in (iii) or the mixture obtained in (iv);
(vi) optionally, film-coating the tablets obtained in (v).

In one embodiment, the present invention relates to a pharmaceutical composition prepared, obtainable, or obtained by a process comprising:
(i) mixing Compound A or a pharmaceutically acceptable salt thereof, the crystalline form of Compound A of the present invention, or the composition comprising the crystalline form of Compound A of the present invention, with at least one filler and at least one binder;
(ii) subjecting the mixture obtained in (i) to a wet granulation comprising contacting the mixture with water, wherein said water comprises at least one surfactant and optionally at least one binder dissolved in the water;
(iii) drying the granules obtained in (ii);
(iv) optionally, mixing the granules obtained in (iii) with at least one disintegrant and at least one lubricant;
(v) optionally, tableting the granules obtained in (iii) or the mixture obtained in (iv);
(vi) optionally, film-coating the tablets obtained in (v).

In another embodiment, the present invention relates to a pharmaceutical composition prepared, obtainable, or obtained by a process comprising:
(i) mixing Compound A or a pharmaceutically acceptable salt thereof, the crystalline form of Compound A of the present invention, or the composition comprising the crystalline form of Compound A of the present invention, with microcrystalline cellulose, mannitol and hypromellose;
(ii) subjecting the mixture obtained in (i) to a wet granulation comprising contacting the mixture with water, wherein said water comprises sodium lauryl sulfate and optionally hypromellose dissolved in the water;
(iii) drying the granules obtained in (ii);
(iv) optionally, mixing the granules obtained in (iii) with crospovidone and magnesium stearate;
(v) optionally, tableting the granules obtained in (iii) or the mixture obtained in (iv);
(vi) optionally, film-coating the tablets obtained in (v).

### Medical Use and Dosage Regimen

In one aspect, the present invention relates to the crystalline form of Compound A of the present invention, the composition of Compound A comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition of Compound A comprising the crystalline form of Compound A of the present invention, for use as a medicament.

In yet another aspect, the present invention relates to the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention, for use in the treatment of cancer.

In one embodiment, the present invention relates to the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention, for use in the treatment of cancer, wherein a predetermined and/or effective amount of the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention is to be administered to a subject intermittently in at least three consecutive doses and the period between each two consecutive doses is at least three weeks and no longer than sixty days.

In another embodiment, the present invention relates to the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention, for use in the treatment of cancer, wherein a predetermined and/or effective amount of the crystalline form of Compound A of the present invention, the composition comprising the crystalline form of Compound A of the present invention, the pharmaceutical composition comprising the crystalline form of Compound A of the present invention, or the pharmaceutical composition comprising the composition comprising the crystalline form of Compound A of the present invention is to be administered to a subject intermittently and the period between consecutive administrations is three weeks.

In yet another aspect, the present invention relates to the use of the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention in the manufacture of a medicament for use in the treatment of cancer.

In one embodiment, the present invention relates to the use of the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention in the manufacture of a medicament for use in the treatment of cancer, wherein a predetermined and/or effective amount of the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention is to be administered to a subject intermittently in at least three consecutive doses and the period between each two consecutive doses is at least three weeks and no longer than sixty days.

In another embodiment, the present invention relates to the use of the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention in the manufacture of a medicament for use in the treatment of cancer, wherein a predetermined and/or effective amount of the crystalline form of Compound A of the present invention, the composition comprising the crystalline form of Compound A of the present invention, the pharmaceutical composition comprising the crystalline form of Compound A of the present invention, or the pharmaceutical composition comprising the composition comprising the crystalline form of Compound A of the present invention is to be administered to a subject intermittently and the period between consecutive administrations is three weeks.

In another aspect, the present invention relates to a method of treating cancer, said method comprising administering a predetermined and/or effective amount of the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention, to a subject in need of such a treatment.

In one embodiment, the present invention relates to a method of treating cancer, said method comprising administering a predetermined and/or effective amount of the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention, to a subject in need of such a treatment, wherein the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention is to be administered to the subject in at least three consecutive doses and the period between each two consecutive doses is at least three weeks and no longer than sixty days.

In another embodiment, the present invention relates to a method of treating cancer, said method comprising administering a predetermined and/or effective amount of the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention, to a subject in need of such a treatment, wherein the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention is to be administered to the subject intermittently and the period between the consecutive administrations is three weeks.

In one embodiment, the predetermined and/or effective amount of the crystalline form of Compound A of the present invention, the composition comprising the crystalline form of Compound A of the present invention, the pharmaceutical composition comprising the Compound A or a pharmaceutically acceptable salt thereof, the pharmaceutical composition comprising the crystalline form of Compound A of the present invention, or the pharmaceutical composition comprising the composition comprising the crystalline form of Compound A of the present invention to be administered to the subject may be in the range of from 1 to 200 mg, preferably of from 5 to 100 mg, and most preferably of from 10 to 60 mg, such as 10 to 45 mg, calculated as free Compound A. For example, the predetermined and/or effective amount to be administered to the subject is selected from the group consisting of 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg and 200 mg, calculated as free Compound A. In a preferred embodiment, the predetermined and/or effective amount to be administered to the subject is selected from the group consisting of 1 mg, 5 mg, 10 mg, 20 mg, 30 mg, 45 mg, 50 mg and 60 mg, calculated as free Compound A. In a particularly preferred embodiment, the predetermined and/or effective amount to be administered to the subject is selected from the group consisting of 10 mg, 20 mg, 30 mg and 45 mg, calculated as free Compound A. In the most preferred embodiment, the predetermined and/or effective amount to be administered to the subject is 30 mg or 45 mg, calculated as free Compound A.

In one embodiment, the cancer to be treated is selected from the group consisting of bladder cancer, breast cancer, brain cancer, head and neck cancer, liver cancer, oral cancer, biliary tract cancer, acute and chronic lymphoid leukemia, acute and chronic myeloid leukemia, chronic myelomonocytic leukemia, colorectal cancer, gastric cancer, gastrointestinal stromal cancer, hepatocellular cancer, glioma, lymphoma, melanoma, multiple myeloma, myeloproliferative disease, neuroendocrine cancer, lung cancer, non-small cell lung cancer (NSCLC), pancreatic cancer, ovarian cancer, prostate cancer, renal cell cancer, sarcoma, and thyroid cancer.

In a preferred embodiment, the cancer to be treated is soft tissue sarcoma (STS). In a specific embodiment, the cancer to be treated is selected from the group consisting of liposarcoma (LPS) including well differentiated liposarcoma (WDLPS) and dedifferentiated liposarcoma (DDLPS). In another preferred embodiment, the cancer to be treated is selected from the group consisting of high-grade leiomyosarcoma, leiomyosarcoma (LMS), gastrointestinal stromal tumor (GIST), adenosarcoma, dermatofibrosarcoma, osteosarcoma, rhabdomyosarcoma, undifferentiated pleomorphic sarcoma (UPS), chondrosarcoma, myxoid chondrosarcoma, myxoid liposarcoma, myxofibrosarcoma (MFS), synovial sarcoma, uterine adenosarcoma, and undifferentiated/unclassified STS.

In still another preferred embodiment, the cancer to be treated is selected from the group consisting of biliary tract cancer, biliary adenocarcinoma, biliary tract adenocarcinoma, pancreatic adenocarcinoma, urothelial carcinoma, intrahepatic cholangiocarcinoma, gastric adenocarcinoma, non-small cell lung cancer (NSCLC), endometrial cancer, colorectal cancer, glioblastoma and melanoma.

In yet another preferred embodiment, the cancer to be treated is selected from the group consisting of biliary tract cancer, intrahepatic cholangiocarcinoma, extrahepatic cholangiocarcinoma, gall bladder carcinoma and ampullary carcinoma.

In a further preferred embodiment, the cancer to be treated is biliary tract cancer, biliary adenocarcinoma or biliary tract adenocarcinoma. In a further preferred embodiment, the cancer to be treated is selected from the group consisting of lung cancer, non-small cell lung cancer (NSCLC), lung adenocarcinoma and non-small cell lung adenocarcinoma.

In a further preferred embodiment, the cancer to be treated is urothelial carcinoma.

In a further preferred embodiment, the cancer to be treated is selected from the group consisting of bladder cancer, kidney cancer, cancer of the renal pelvis and cancer of the ureter.

In a further preferred embodiment, the cancer to be treated is urothelial bladder cancer.

In a further preferred embodiment, the cancer to be treated is selected from the group consisting of breast cancer, ovarian cancer and endometrial cancer.

In a further preferred embodiment, the cancer to be treated is pancreatic cancer, preferably pancreatic ductal adenocarcinoma (PDAC).

In another embodiment, the cancer to be treated is a p53 wildtype form of cancer.

In another embodiment, the cancer to be treated is an MDM2 amplified form of cancer.

In another embodiment, the cancer to be treated is a p53 wildtype and MDM2 amplified form of cancer.

In another embodiment, the cancer to be treated is a form of cancer which is not MDM2 amplified.

In another embodiment, the cancer to be treated is a p53 wildtype form of cancer which is not MDM2 amplified.

In a further embodiment, the cancer to be treated is an unresectable locally advanced or metastatic dedifferentiated liposarcoma (DDLPS). In one embodiment the treatment is a first line treatment. In another embodiment the treatment is a second or later line treatment. In preferred embodiments the patient is an adult patient.

In a further embodiment, the cancer to be treated is advanced or metastatic p53 wild-type undifferentiated pleomorphic sarcoma (UPS) or myxofibrosarcoma (MFS) and the treatment is in combination with ezabenlimab. In one embodiment the treatment is a first line treatment. In another embodiment the treatment is a second or later line treatment where the patient has progressed on one or more prior systemic therapy for advanced or metastatic disease. In preferred embodiments the patient is an adult patient.

In a further embodiment, the cancer to be treated is unresectable locally advanced or metastatic MDM2 amplified and p53 wild-type biliary tract adenocarcinoma. In one embodiment the treatment is first line treatment. In another embodiment the patient has progressed following earlier treatment or has no satisfactory alternative treatments. In preferred embodiments the patient is an adult patient.

### EXAMPLES

The following non-limiting examples are illustrative for the disclosure and are not to be construed as to be in any way limiting for the scope of the invention.

### Example 1: Preparation of the Crystalline Form of Compound a of the Present Invention from Solvent System IPA/H₂O

Compound A (10 g, crude material e.g. prepared as disclosed in WO 2017/060431 A1) was dissolved in IPA (51 mL) at a temperature of 75° C. The obtained solution was filtrated while hot and the filter was rinsed with a hot mixture of IPA/H₂O 60:40 (w/w) (10 mL). Hot water (27 mL) was added to the solution which was thereafter cooled to 70° C. The solution was seeded with the crystalline form of Compound A of the present invention (200 mg, e.g. prepared according to any one of Examples 2-1, 2-2 or 4) whereupon a suspension was obtained, which was stirred at 70° C. for 15 minutes. After slow addition of an IPA/H₂O 35:65 (w/w) mixture (88 mL) over the course of 1 hour, the suspension was cooled to 20° C. in 2 hours and stirred overnight (approximately 16 hours) at the same temperature. The obtained solid was collected by filtration, washed with H₂O (3×50 mL) and dried at 75° C. for 24 hours to obtain 9.2 g of the crystalline form of Compound A of the present invention.

### Example 2: Preparation of the Crystalline Form of Compound a of the Present Invention from Solvent Systems EtOH/H₂O and n-PrOH/H₂O

Compound A (approximately Ig, see Table 1 below for the exact amounts employed, e.g. prepared as disclosed in WO 2017/060431 A1) was dissolved in the solvent (3 mL) indicated in Table 1 upon heating to a temperature of about 60° C. while magnetically stirring at 400 rpm.

To the obtained solution water was added until the solution became cloudy (see Table 1 for the amount water added). The thus obtained mixture was allowed to naturally cool to RT and slurried for 3 days. The obtained solid was collected by filtration and dried at ambient conditions in a fume hood for 72 hours.

TABLE 1
Crystallization from different aqueous alcohols

| Example | Amount Compound A | Solvent | Volume H₂O |
|---|---|---|---|
| 2-1 | 1091 mg | EtOH | 8 mL |
| 2-2 | 1078 mg | n-PrOH | 8 mL |

### Example 3: Preparation of the Crystalline Form of Compound a of the Present Invention from Solvent System ACN/H₂O

Compound A (8 g, crude material e.g. prepared as disclosed in WO 2017/060431 A1) was dissolved in a mixture of ACN (32 mL) and H₂O (8 mL) at a temperature of 80° C. The obtained clear solution was cooled to 50° C. and seeded with the crystalline form of Compound A of the present invention (100 mg, e.g. prepared according to any one of Examples 2-1, 2-2 or 4) whereupon a suspension was obtained. After the addition of another portion of water (16 mL) the slurry became unstirrable, that's why the temperature was again increased to 75° C. and the suspension further stirred at this temperature for about 16 hours. Thereafter, the obtained solid was collected by filtration, washed with H₂O (3×40 mL) and dried at 75° C. for 24 hours.

### Example 4: Preparation of the Crystalline Form of Compound a of the Present Invention from Solvent System ACT/H₂O

Compound A (964 mg, e.g. prepared as disclosed in WO 2017/060431 A1) was dissolved in acetone (3 mL) upon heating to a temperature of about 60° C. while magnetically stirring at 400 rpm. To the obtained solution water (2 mL) was added until the solution became cloudy. The thus obtained mixture was allowed to naturally cool to RT and slurried for 3 days. The obtained solid was collected by filtration and dried at ambient conditions in a fume hood for 72 hours.

### Example 5: X-Ray Powder Diffraction

X-ray powder diffraction was performed on a Bruker D8 Advance X-Ray diffractometer (Cu-Kalpha radiation, wavelength 0.15406 nm). The sample was lightly ground using a metal spatula inside the vial and packed in a small area silicon holder. The diffraction profile was collected from 2-35° 2-theta with a step size of 0.05°/step, and a collection time of 1 second/step. A typical precision of the 2-theta values is in the range of ±0.2° 2-theta, preferably in the range of ±0.1° 2-theta. Thus a reflection that usually appears at 9.3° 2-theta for example can appear between 9.1 and 9.5°2-theta, preferably between 9.2 and 9.4°2-theta on most X-ray diffractometers under standard conditions.

The diffractogram of the crystalline form of Compound A of the present invention obtained from solvent system IPA/1H₂O in Example 1 hereinabove is displayed in FIG. 1. The corresponding list of reflections (peaks) is provided in the following Table 2.

TABLE 2
Reflection (peak) positions of the crystalline form of
Compound A of the present invention obtained from Example 1
Reflection (peak) position
[°2-theta]

4.4
5.1
6.6
8.7
9.3
11.9
13.2
13.8
14.2
14.6
17.1
18.6
19.9
21.9
23.0

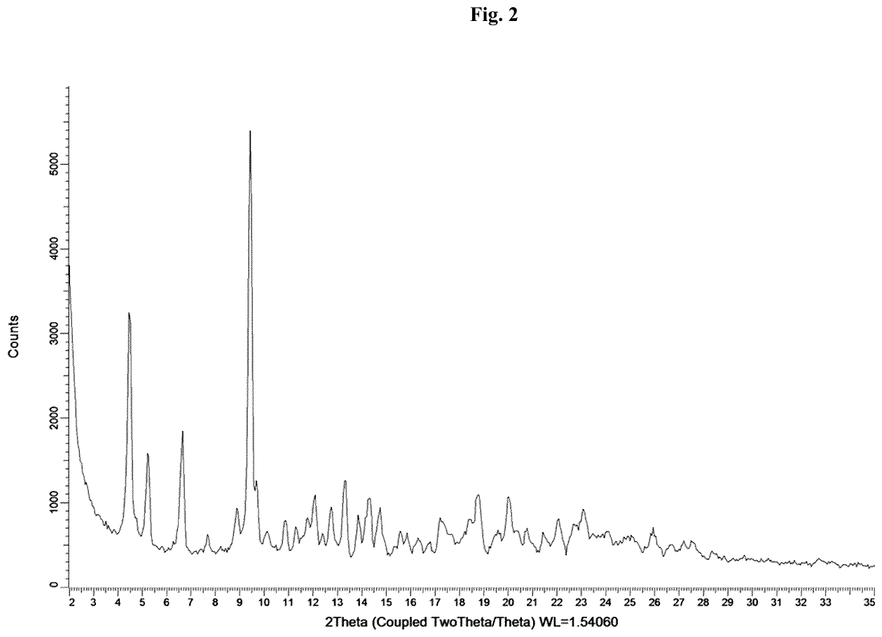

The diffractogram of the crystalline form of Compound A of the present invention obtained from solvent system EtOH/H₂O in Example 2-1 hereinabove is displayed in FIG. 2. The corresponding list of reflections (peaks) is provided in the following Table 3.

TABLE 3
Reflection (peak) positions of the crystalline form of Compound
A of the present invention obtained from Example 2-1
Reflection (peak) position
[°2-theta]

4.5
5.2
6.7
7.7
8.9
9.4
10.9
12.1
12.7
13.3
14.3
14.7
15.6
17.2
18.4
18.8
19.6
20.0
20.8
22.1
22.7
23.1
25.9
27.2
27.5

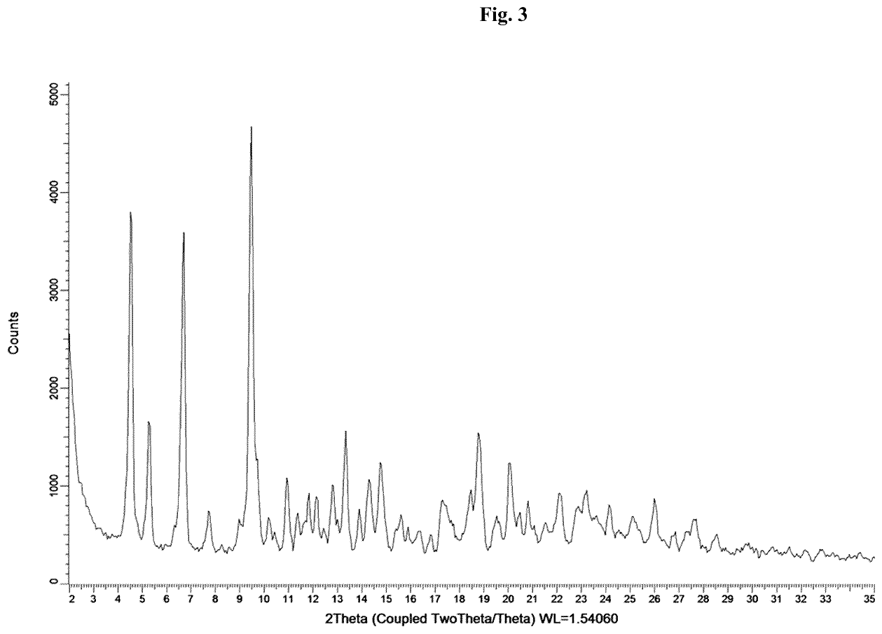

The diffractogram of the crystalline form of Compound A of the present invention obtained from solvent system n-PrOH/H₂O in Example 2-2 hereinabove is displayed in FIG. 3. The corresponding list of reflections (peaks) is provided in the following Table 4.

TABLE 4
Reflection (peak) positions of the crystalline form of Compound
A of the present invention obtained from Example 2-2
Reflection (peak) position
[°2-theta]

4.5
5.3
6.7
7.7
9.5
10.9
11.8
12.8
13.3
14.3
14.8
15.6
17.3
18.5
18.8
19.5
20.1
20.8
21.5
22.1
22.9
23.2
23.6
24.2
25.1

The diffractogram of the crystalline form of Compound A obtained from solvent system ACN/1H₂O in Example 3 hereinabove is displayed in FIG. 4. The corresponding list of reflections (peaks) is provided in the following Table 5.

TABLE 5
Reflection (peak) positions of the crystalline form of Compound
A of the present invention obtained from Example 3
Reflection (peak) position
[°2-theta]

4.2
5.3
6.3
6.7
7.6
8.2
9.6
10.3
11.2
12.0
13.4
13.8
14.4
14.9
15.5
16.3
17.1
17.6
18.0
18.6
19.0
19.4
20.0
20.4
20.8
21.5
22.1
22.5
23.8
24.3
25.1
26.8
27.7

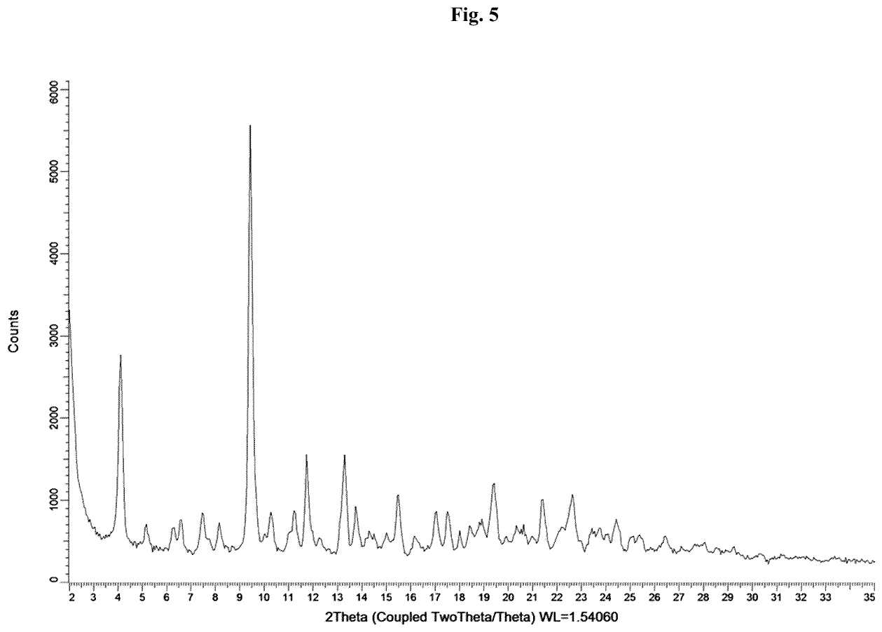

The diffractogram of the crystalline form of Compound A obtained from solvent system ACT/1H₂O in Example 4 hereinabove is displayed in FIG. 5. The corresponding list of reflections (peaks) is provided in the following Table 6.

TABLE 6
Reflection (peak) positions of the crystalline form of Compound
A of the present invention obtained from Example 4
Reflection (peak) position
[°2-theta]

4.1
5.2
6.3
6.6
7.5
8.2
9.4
10.3
11.2
11.7
12.3
13.3
13.8
14.3
15.5
16.2
17.0
17.5
18.9
19.4
21.4
22.2
22.6
23.4
24.4
25.4
26.4

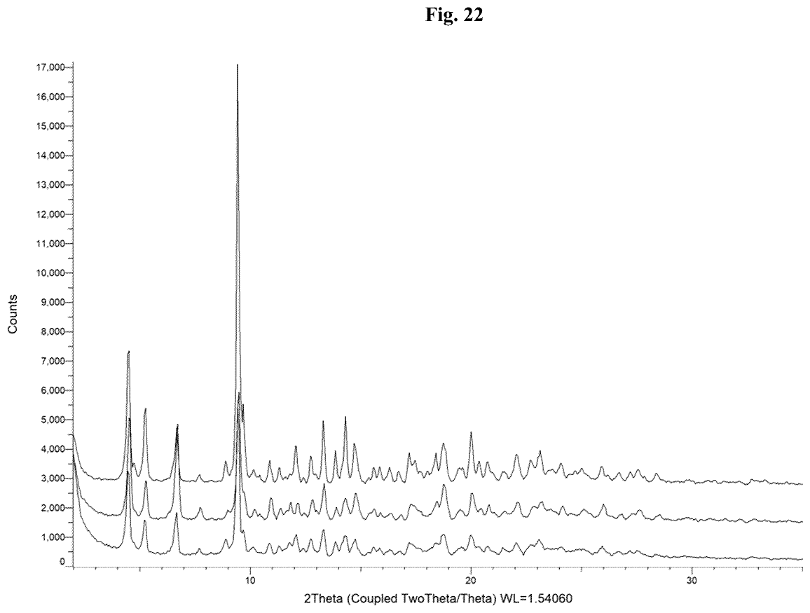

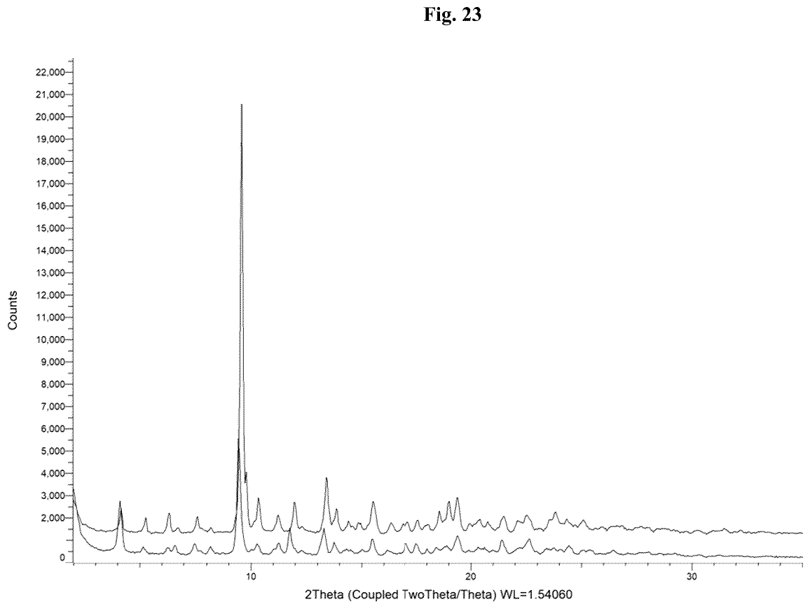

In addition, FIG. 22 and FIG. 23 provide comparisons of the XRPDs of crystalline Compound A obtained from Examples 1 to 4 hereinabove.

### Example 6: Raman Spectroscopy

Raman spectra were recorded with a transmission Raman spectrometer (TRS100) from Agilent, equipped with a thermoelectrically cooled charge-coupled detector. Data was acquired over spectral range 38-2400 cm⁻¹ using an 830 nm enhanced photo diode excitation laser at a power setting of 500 mW, spot size of 4 mm and spectral resolution of <8 cm⁻¹. A typical precision of the wavenumber values is in the range of about ±2 cm⁻¹. Thus, a Raman peak that appears at 1251 cm⁻¹ can appear between 1249 and 1253 cm⁻¹ on most Raman spectrometers under standard conditions.

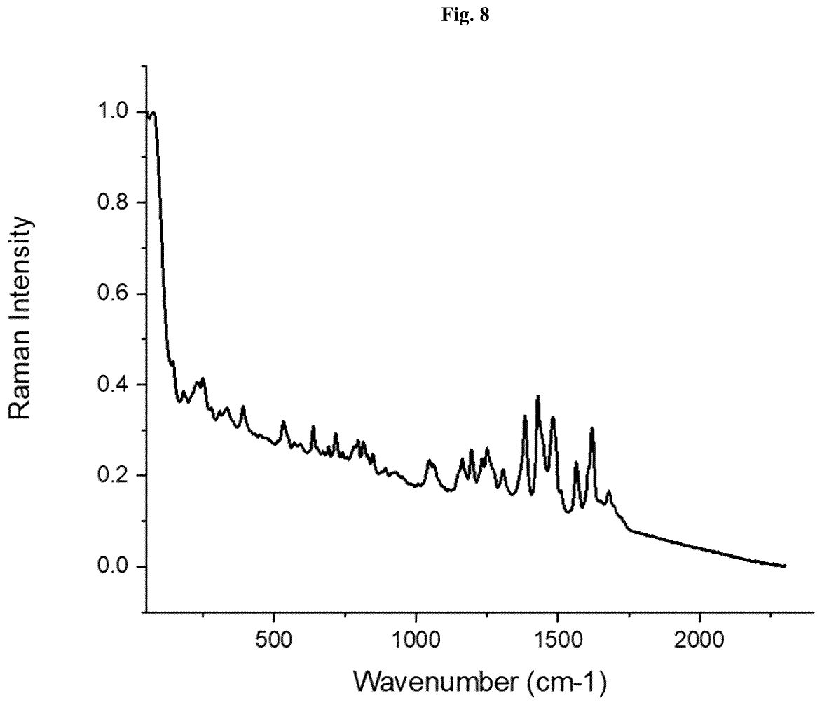

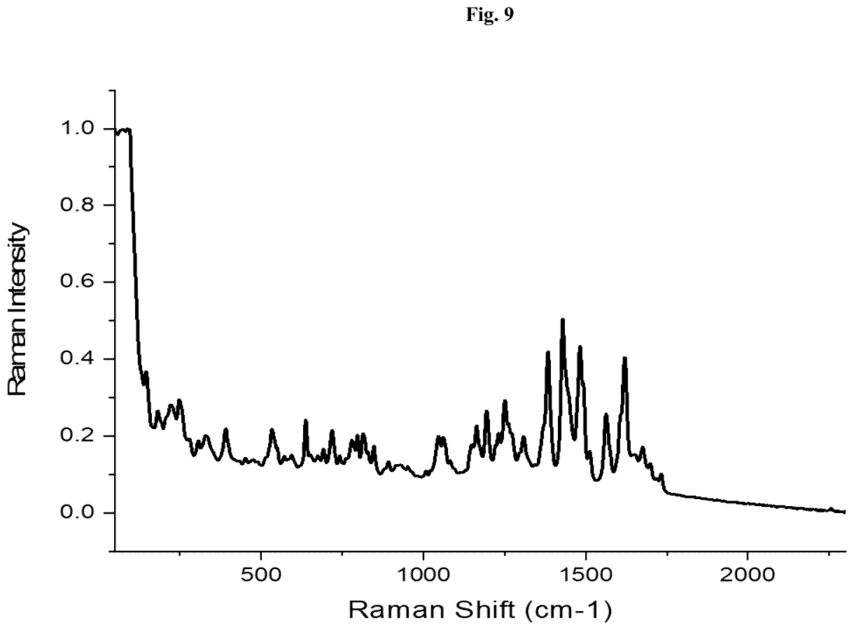

The Raman spectra of the crystalline forms of Compound A of the present invention obtained from solvent system IPA/H₂O in Example 1 and from solvent system ACN/H₂O in Example 3 hereinabove are displayed in FIG. 8 and FIG. 9, respectively.

### Example 7: Solid-State NMR

¹³C ssNMR data were acquired on a Bruker Avance NEO NMR spectrometer (Bruker Biospin, Inc., Billerica, MA) with a 9.4 T magnet (¹H=400.46 MHz, ¹³C=100.7 MHz). Samples were packed in 3.2 mm O.D. zirconia rotors with Vespel® drive tips. A Bruker model 3.2BL BB probe was used for data acquisition and sample spinning about the magic-angle (54.74 degrees) with a spinning rate of 22 kHz with variable temperature control adjusted so that the internal sample temperature was situated at ambient temperature and pressure. A standard cross-polarization pulse sequence was used with a ramped Hartman-Hahn match pulse on the proton channel. The pulse sequence used an 8 millisecond contact pulse for the sample obtained from the solvent system ACN/H₂O according to Example 3 and an 11 millisecond contact pulse for the sample obtained from IPA/H₂O according to Example 1. A 5 second recycle delay was used for the sample obtained from solvent system ACN/H₂O according to Example 3 and an 11 second recycle delay was used for the sample obtained from IPA/H₂O according to Example 1. TPPM decoupling during acquisition was also employed in the pulse sequence. No exponential line broadening was used prior to Fourier transformation of the free induction decay. Chemical shifts were referenced using the secondary standard of adamantane, with the high frequency resonance being set to 38.48 ppm. The magic-angle was set using the ⁷⁹Br signal from KBr powder at a spinning rate of 5 kHz. A typical precision of the chemical shifts is in the range of about ±0.3 ppm. Thus, a peak that appears at 176.4 ppm can appear between 176.1 and 176.7 ppm on most ssNMR spectrometers under standard conditions.

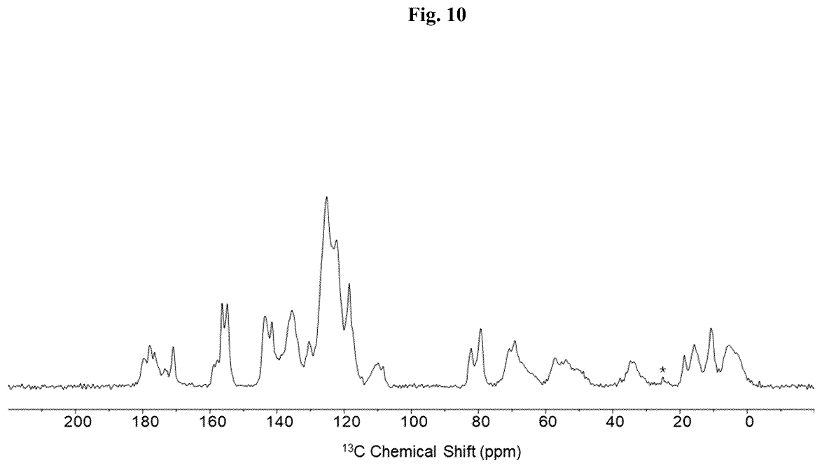

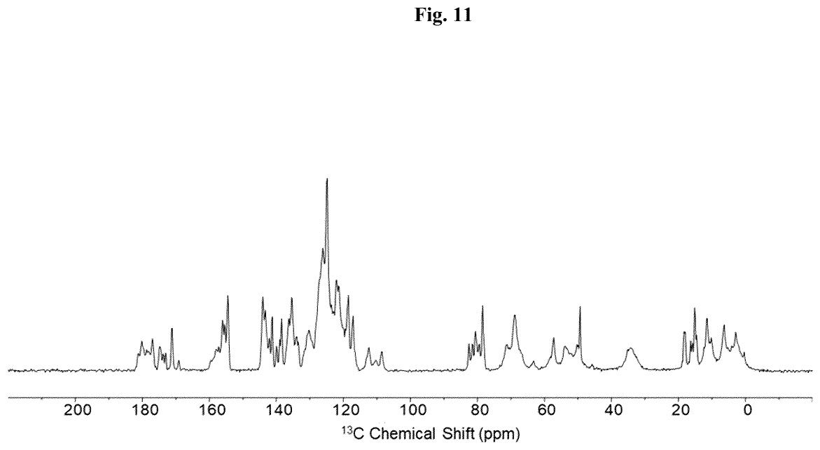

¹³C-ssNMR spectra of the crystalline form of Compound A of the present invention obtained from the solvent system IPA/H₂O according to Example 1 and from the solvent system ACN/H₂O according to Example 3 hereinabove are displayed in FIG. 10 and FIG. 11, respectively.

¹⁹F ssNMR data were acquired on a Bruker Avance NEO NMR spectrometer (Bruker Biospin, Inc., Billerica, MA) with a 9.4 T magnet (¹H=400.46 MHz, ¹⁹F=376.76 MHz). Samples were packed in 3.2 mm O.D. zirconia rotors with Vespel® drive tips. A Bruker model 3.2BL BB probe was used for data acquisition and sample spinning about the magic-angle (54.74 degrees). Sample spectra were acquired with a spinning rate of 22 kHz with variable temperature control adjusted so that the internal sample temperature was situated at ambient temperature and pressure. A standard spin echo pulse sequence was used with a 30 seconds recycle delay for the sample obtained from ACN/H₂O according to Example 3 and a 20.8 seconds recycle delay for the sample obtained from IPA/H₂O according to Example 1. TPPM 1H decoupling was also employed. No exponential line broadening was used prior to Fourier transformation of the free induction decay. Chemical shifts were referenced using the most intense signal from polyvinylidene fluoride (PVDF), with the resonance set to −91.0 ppm. The magic-angle was set using the ⁷⁹Br signal from KBr powder at a spinning rate of 5 kHz. A typical precision of the chemical shifts is in the range of about ±0.5 ppm. Thus, a peak that appears at −117.5 ppm can appear between −118.0 and −117.0 ppm on most ssNMR spectrometers under standard conditions.

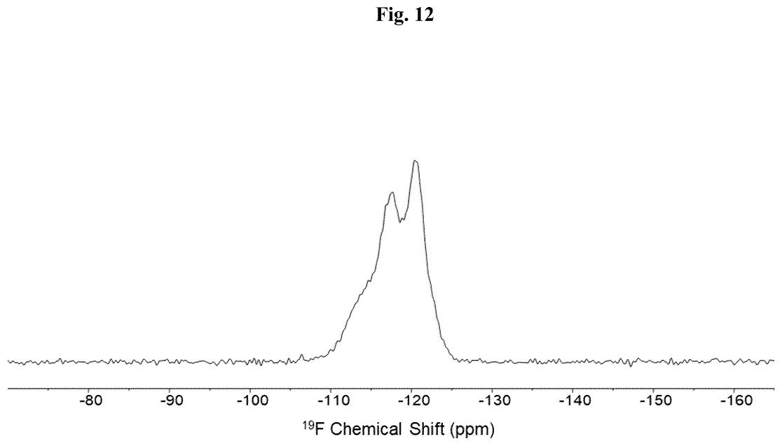

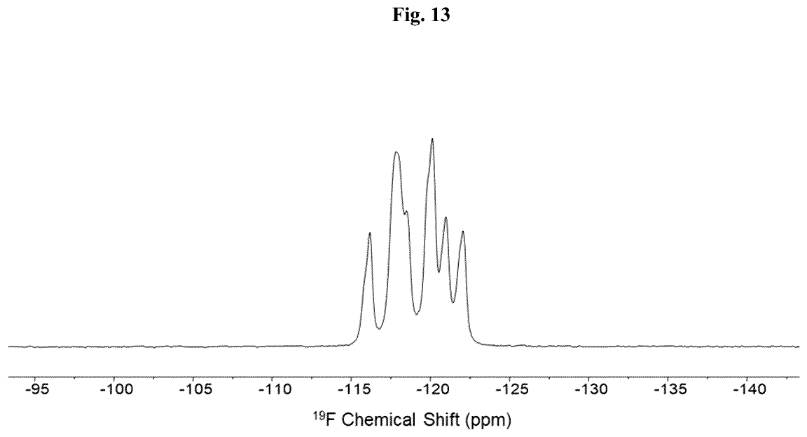

¹⁹F-ssNMR spectra of the crystalline form of Compound A of the present invention obtained from the solvent system IPA/H₂O according to Example 1 and from the solvent system ACN/H₂O according to Example 3 hereinabove are displayed in FIG. 12 and FIG. 13, respectively.

### Example 8: Accelerated Stress Tests

Crystalline materials of Compound A of the present invention were exposed to various temperature and humidity stress conditions and investigated by XRPD and HPLC in order to investigate their physical and chemical stability.

### Example 8.1: Stability at 40° C./75% RH

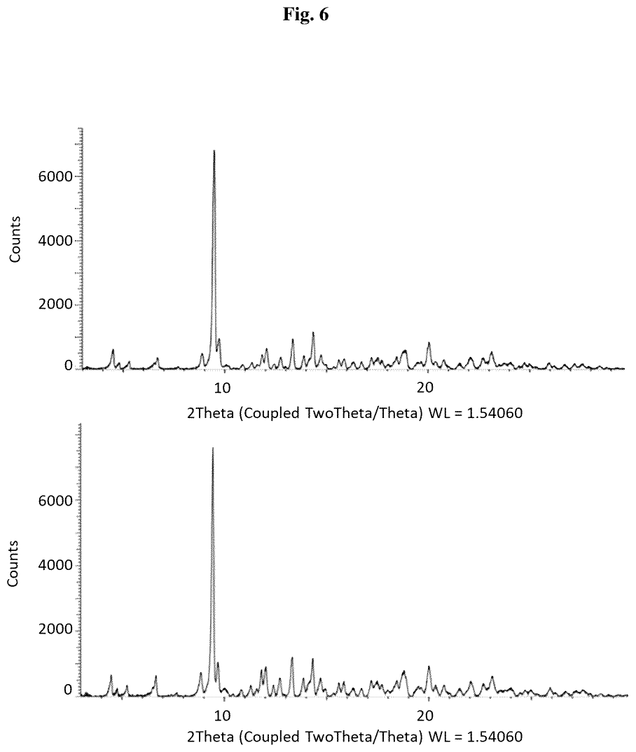

The crystalline form of Compound A of the present invention (prepared according to the procedure described in Example 1) was open stored under heat and humidity stress conditions of 40° C. and 75% RH for 6 months. No differences in reflection positions were observed for the crystalline form of Compound A of the present invention, when comparing the XRPD of the initial sample with the XRPD of the stressed sample, as can be seen from FIG. 6, proofing its physical stability.

In addition, no significant increase of chemical impurities was observed for the crystalline form of Compound A of the present invention, when comparing the impurity profile of the initial sample with the impurity profile of the stressed sample as can be seen from Table 7, proofing its chemical stability.

TABLE 7
Chemical stability of the crystalline form of Compound
A of the present invention at 40°C/75% RH

| | Initial Peak Area-% | Peak Area-% at 6 months |
|---|---|---|
| Impurity A | 0.06% | Not detected |
| Impurity B | 0.06% | 0.06% |
| Impurity C | 0.05% | 0.06% |
| Impurity D | 0.23% | 0.25% |
| Impurity E | 0.31% | 0.33% |
| Total impurities | 0.71 % | 0.70% |

### Example 8.2: Stability at 25° C./60% RH

The crystalline form of Compound A of the present invention (prepared according to the procedure described in Example 1) was open stored under humidity stress conditions of 25° C. and 60% RH for 6 months. No differences in reflection positions were observed for the crystalline form of Compound A of the present invention, when comparing the XRPD of the initial sample with the XRPD of the stressed sample.

In addition, no significant increase of chemical impurities was observed when comparing the impurity profile of the initial samples with the impurity profile of the stressed samples as can be seen from Table 8, proofing its chemical stability.

TABLE 8
Chemical stability at 25°C/60% RH, open storage

| | Start | 1 month | 2 months | 3 months | 6 months |
|---|---|---|---|---|---|
| Impurity 1 | 0.08% | 0.07% | 0.07% | 0.07% | 0.07% |
| Impurity 2 | 0.06% | ≤0.05% | ≤0.05% | ≤0.05% | ≤0.05% |
| Impurity 3 | ≤0.05% | 0.06% | 0.06% | ≤0.05% | 0.06% |
| Impurity 4 | 0.26% | 0.23% | 0.23% | 0.23% | 0.23% |
| Impurity 5 | 0.26% | 0.25% | 0.25% | 0.25% | 0.27% |
| Total impurities | 0.66% | 0.61% | 0.61% | 0.60% | 0.63% |

### Example 8.3: Stability at 60° C./80% RH

The crystalline form of Compound A of the present invention (prepared according to the procedure described in Example 1 and 3, respectively) was open stored under heat and humidity stress conditions of 60° C. and 80% RH for 3 days. No differences in reflection positions were observed for the crystalline form of Compound A of the present invention, when comparing the XRPD of the initial samples with the XRPD of the stressed samples.

In addition, no significant increase of chemical impurities was observed when comparing the impurity profiles of the initial samples with the impurity profiles of the stressed samples. For example, the increase in total impurities of crystalline compound A obtained according to the procedure of Example 3 was below 0.5%. Table 9, provides the chemical stability data of the crystalline compound A obtained according to the procedure of Example 1. As can be seen the material remained chemically stable.

TABLE 9
Chemical stability at 60°C/80% RH, open storage

| | Start | 3 days |
|---|---|---|
| Impurity 1 | 0.08% | 0.07% |
| Impurity 2 | 0.06% | ≤0.05% |
| Impurity 3 | ≤0.05% | 0.07% |
| Impurity 4 | 0.26% | 0.24% |
| Impurity 5 | 0.26% | 0.31% |
| Total impurities | 0.66% | 0.69% |

### Example 8.4: Stability at 70° C. and 105° C.

The crystalline form of Compound A of the present invention (prepared according to the procedure described in Example 1) was stored in closed glass containers at heat stress condition of 70° C. for 3 weeks. In addition, the crystalline form of Compound A of the present invention (prepared according to the procedures described in Example 1 and 3, respectively) was stored in closed glass containers at heat stress condition of 105° C. for 24 hours. No differences in reflection positions were observed for the crystalline form of Compound A of the present invention, when comparing the XRPD of the initial samples with the XRPD of the stressed samples.

In addition, no significant increase of chemical impurities was observed for the materials when comparing the impurity profiles of the initial samples with the impurity profiles of the stressed samples. For example, the increase in total impurities of crystalline compound A obtained according to the procedure of Example 3 was below 0.5%. Table 10, provides the chemical stability data of the crystalline compound A obtained according to the procedure of Example 1. As can be seen the material remained chemically stable at all stress conditions.

TABLE 10
Chemical stability at 70° C. and 105° C.

| | | 70° C. | | | 105° C. |
|---|---|---|---|---|---|
| | Start | 1 week | 2 weeks | 3 weeks | 24 hours |
| Impurity 1 | 0.08% | 0.07% | 0.07% | 0.07% | 0.07% |
| Impurity 2 | 0.06% | ≤0.05% | ≤0.05% | ≤0.05% | ≤0.05% |
| Impurity 3 | ≤0.05% | 0.07% | 0.06% | 0.07% | 0.06% |
| Impurity 4 | 0.26% | 0.24% | 0.23% | 0.24% | 0.23% |
| Impurity 5 | 0.26% | 0.26% | 0.25% | 0.31% | 0.25% |
| Total Imp. | 0.66% | 0.64% | 0.61% | 0.69% | 0.61% |

### Example 9: Gravimetric Moisture Sorption

The crystalline form of Compound A of the present invention was subjected to a gravimetric moisture sorption experiment using a DVS intrinsic moisture sorption analyzer (Surface Measurement System). The measurement was conducted in relative humidity steps of 10%. The time per step was set to a minimum of 5 minutes and a maximum of 6 hours. If an equilibrium condition with a constant mass of ±0.02% within 10 min was reached before the maximum time, the subsequent step was applied before the maximum time of 6 hours. If no equilibrium was achieved the consecutive humidity step was applied after the maximum time of 6 hours. The temperature was (25.0±0.1° C.)

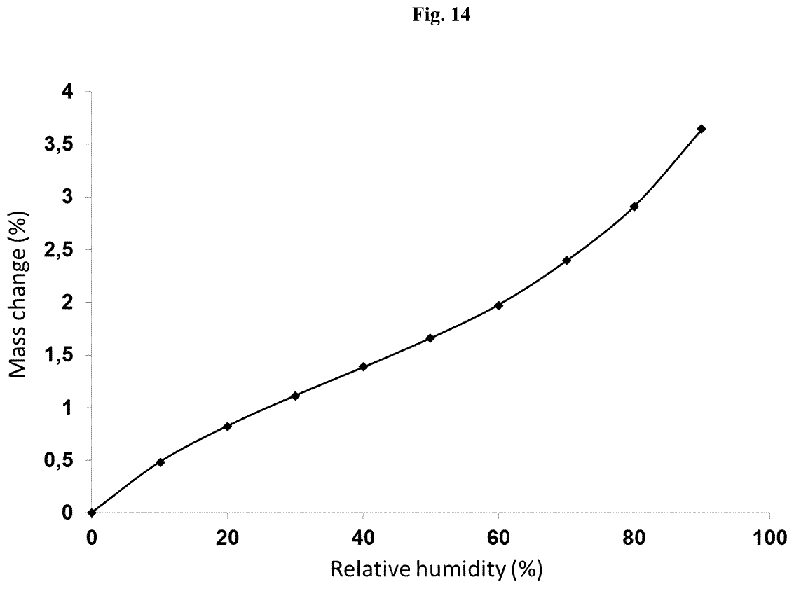

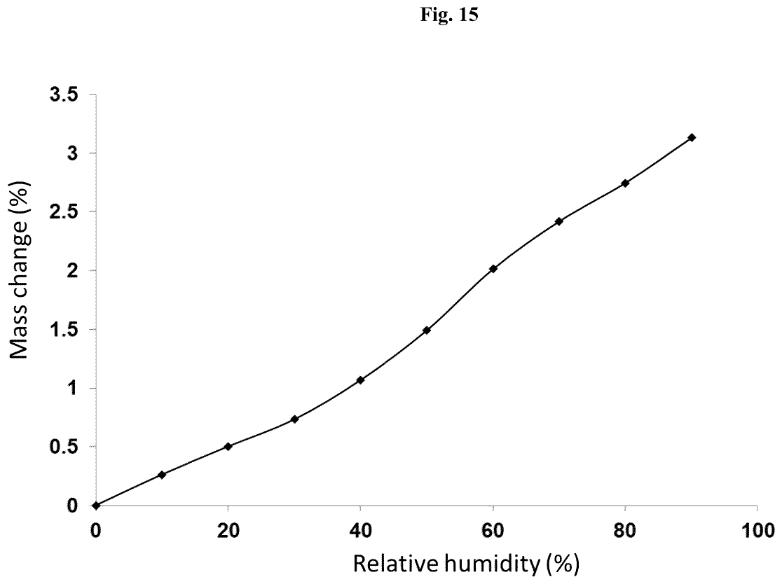

The moisture sorption curves of the crystalline form of Compound A of the present invention obtained from IPA/H₂O according to Example 1 and obtained from ACN/H₂O according to Example 3 hereinabove are displayed in FIG. 14 and FIG. 15, respectively.

As can be seen from FIG. 14 the crystalline form of Compound A of the present invention obtained from IPA/H₂O took up about 3.6% (w/w) of water in the range of 0 to 90% relative humidity. FIG. 15 indicates a water uptake of about 3.1% (w/w) in the range of 0 to 90% relative humidity for the crystalline form of Compound A of the present invention obtained from ACN/H₂O.

### Example 10: Film-Coated Tablet Formulations and their Preparation

Hypromellose and sodium lauryl sulfate are dissolved in purified water to produce the granulation liquid. Mannitol, Compound A (e.g. the crystalline form of the present invention prepared according to Example 1 herein) and microcrystalline cellulose are pre-screened and granulated in a suitable granulator, using the granulation liquid. After optional wet screening, the granules are dried in a suitable drier, followed by dry screening. The screened granules are blended with crospovidone in a suitable diffusion blender. Pre-screened magnesium stearate is added and blended in a suitable diffusion blender. The final blend is compressed into tablet cores. The film-coating mixture is dispersed in purified water and the tablet cores are coated with the film-coating suspension in a suitable pan coater.

TABLE 11
Qualitative and quantitative compositions of 1 mg and 5 mg FCTs

| | Formulation | | |
|---|---|---|---|
| | F1 | F2 | |
| | 1 mg | 5 mg | — |
| Ingredients | [mg/tablet] | [m/tablet] | Function |
| Compound A | 1.00 | 5.00 | API |
| Mannitol | 47.91 | 239.55 | Filler |
| MCC | 13.40 | 67.00 | Filler |
| Hypromellose | 1.34 | 6.70 | Binder |
| Sodium lauryl sulfate | 0.34 | 1.68 | Surfactant |
| Crospovidone | 2.35 | 11.73 | Disintegrant |
| Magnesium stearate | 0.67 | 3.35 | Lubricant |
| Water, purified* | q.s. | q.s. | Solvent |
| Sub-total (tablet core) | 67.00 | 335.0 | — |
| Hypromellose | 1.44 | 3.84 | Film-former |
| Macrogols | 0.42 | 1.12 | Plasticizer |
| Talc | 0.54 | 1.44 | Anti-adherent |
| Titanium dioxide | 0.48 | 1.28 | Pigment |
| Iron oxide red | 0.12 | 0.32 | Pigment |
| Water, purified* | q.s. | q.s. | Solvent |
| Sub-total (film coat) | 3.00 | 8.00 | — |
| Total (FCT) | 70.00 | 343.00 | — |

*Removed during processing, does not appear in the final product

TABLE 12
Qualitative and quantitative compositions of 10 mg and 50 mg FCTs

| | Formulation | | |
|---|---|---|---|
| | F3 | F4 | |
| | 10 mg | 50 mg | |
| Ingredients | [mg/tablet] | [mg/tablet] | Function |
| Compound A | 10.000 | 50.000 | Active ingredient |
| Mannitol | 38.910 | 194.550 | Filler |
| MCC | 13.400 | 67.000 | Filler |
| Hypromellose | 1.340 | 6.700 | Binder |
| Sodium lauryl sulfate | 0.335 | 1.675 | Surfactant |
| Crospovidone | 2.345 | 11.725 | Disintegrant |
| Magnesium stearate | 0.670 | 3.350 | Lubricant |
| Water, purified* | q.s. | q.s. | Solvent |
| Total (tablet core) | 67.00 | 335.00 | — |
| Hypromellose | 1.44 | 3.84 | Film-former |
| Macrogols | 0.42 | 1.12 | Plasticizer |
| Talc | 0.54 | 1.44 | Anti-adherent |
| Titanium dioxide | 0.48 | 1.28 | Pigment |
| Iron oxide red | 0.12 | 0.32 | Pigment |
| Water, purified* | q.s. | q.s. | Solvent |
| Sub-total (film coat) | 3.00 | 8.00 | — |
| Total (FCT) | 70.00 | 343.00 | — |

*Removed during processing, does not appear in the final product

### Example 11: Oral Granules with and without Surfactant

If present in the formulation, sodium lauryl sulfate is dissolved in purified water to produce the granulation liquid. Mannitol, hypromellose and microcrystalline cellulose are pre-screened, mixed with Compound A (e.g. the crystalline form of the present invention prepared according to Example 1 herein) and granulated with the granulation liquid in a suitable granulator. After optional wet screening, the granules are dried in a suitable drier, followed by dry screening. The screened granules are blended with pre-screened crospovidone in a suitable freefall blender. Pre-screened magnesium stearate is added and blended in a suitable free fall blender.

TABLE 13
Qualitative and quantitative compositions
of granules with and without surfactant

| | Formulation | | |
|---|---|---|---|
| | F5 | F6 | |
| | 45 mg granules | 45 mg granules | |
| Ingredients | with SLS | without surfactant | Function |
| Compound A | 45.00 | 45.00 | API |
| Mannitol | 211.95 | 215.55 | Filler |
| MCC | 74.25 | 74.25 | Filler |
| Hypromellose | 10.80 | 10.80 | Binder |
| SLS | 3.60 | — | Surfactant |
| Crospovidone | 10.80 | 10.80 | Disintegrant |
| Magnesium stearate | 3.60 | 3.60 | Lubricant |
| Water, purified | q.s | q.s | Solvent |
| Total (granules) | 360.00 | 360.00 | — |

### Example 12: Film-Coated Tablet Formulations and their Preparation

Sodium lauryl sulfate is dissolved in purified water to produce the granulation liquid. Mannitol, hypromellose and microcrystalline cellulose are pre-screened, mixed with Compound A (e.g. the crystalline form of the present invention prepared according to Example 1 herein) and granulated with the granulation liquid in a suitable granulator. After optional wet screening, the granules are dried in a suitable drier, followed by dry screening. The screened granules are blended with pre-screened crospovidone in a suitable freefall blender. Pre-screened magnesium stearate is added and blended in a suitable free fall blender. The final blend is compressed into tablet cores. The film-coating mixture is dispersed in purified water and the tablet cores are coated with the film-coating suspension in a suitable pan coater.

TABLE 14
Qualitative and quantitative compositions
of 10 mg, 30 mg and 45 mg FCTs

| | Formulation | | | |
|---|---|---|---|---|
| | F7 | F8 | F9 | |
| | 10 mg | 30 mg | 45 mg | |
| Ingredients | [mg/tablet] | [mg/tablet] | [mg/tablet] | Function |
| Compound A | 10.00 | 30.00 | 45.00 | API |
| Mannitol | 47.50 | 142.50 | 213.75 | Filler |
| MCC | 16.50 | 49.50 | 74.25 | Filler |
| Hypromellose | 2.40 | 7.20 | 10.80 | Binder |
| SLS | 0.40 | 1.20 | 1.80 | Surfactant |
| Crospovidone | 2.40 | 7.20 | 10.80 | Disintegrant |
| Magnesium stearate | 0.80 | 2.40 | 3.60 | Lubricant |
| Water, purified | q.s. | q.s | q.s | Solvent |
| Sub-total | 80.00 | 240.00 | 360.00 | — |
| (tablet core) | | | | |
| Hypromellose | 1.4400 | 3.360 | 4.320 | Film-forming |
| | | | | agent |
| Macrogols | 0.4200 | 0.980 | 1.260 | Plasticizer |
| Talc | 0.5400 | 1.260 | 1.620 | Anti-adherent |
| Titanium dioxide | 0.5925 | 1.120 | 1.440 | Pigment |
| Iron oxide red | — | — | 0.360 | Pigment |
| Iron oxide yellow | 0.0075 | 0.280 | — | Pigment |
| Water, purified | q.s. | q.s. | q.s. | Solvent |
| Sub-total | 3.00 | 7.00 | 9.00 | — |
| (film coat) | | | | |
| Total (FCT) | 83.00 | 247.00 | 369.00 | — |

### Comparative Example 1: Bioaccessibility

A tiny-TIM system was used as a model for the simulation of the physiological processes occurring in human stomach and small intestine to study the in vitro bioaccessibility of the crystalline Compound A of the present invention (prepared according to the procedure described in Example 1) under simulated fasted conditions. Thereby, the impact of drug substance particle size distribution (PSD) and amorphous content on bioaccessibility was evaluated by using tablets manufactured from a range of drug substance batches with different amorphous contents and variations in PSD. The investigated drug products also cover extreme conditions such as low amorphous content and coarse PSD as well as high amorphous content and fine PSD. An overview of the different grades of material, which has been used to prepare the tablets is provided in Table 15.

TABLE 15
Crystalline Compound A drug substance with different
particle size and varying amorphous content
employed for the production of the investigated tablets

| | Tablet | Drug | Amorphous | Drug substance particle |
|---|---|---|---|---|
| Batch | strengths | load | content | size D50 |
| A | 10 mg | 14.9% | 18% | 17.2 μm |
| B | 5 mg | 1.5% | 18% | 17.2 μm |
| C | 10 mg | 14.9% | 12% | 25.0 μm |
| D | 5 mg | 1.5% | 18% | 17.2 μm |
| F | 45 mg | 12.5% | 21% | 8.4 μm |
| G | 10 mg | 12.5% | 75% | 21.0 μm |
| H | 10 mg | 12.5% | 6% | 3.6 μm |

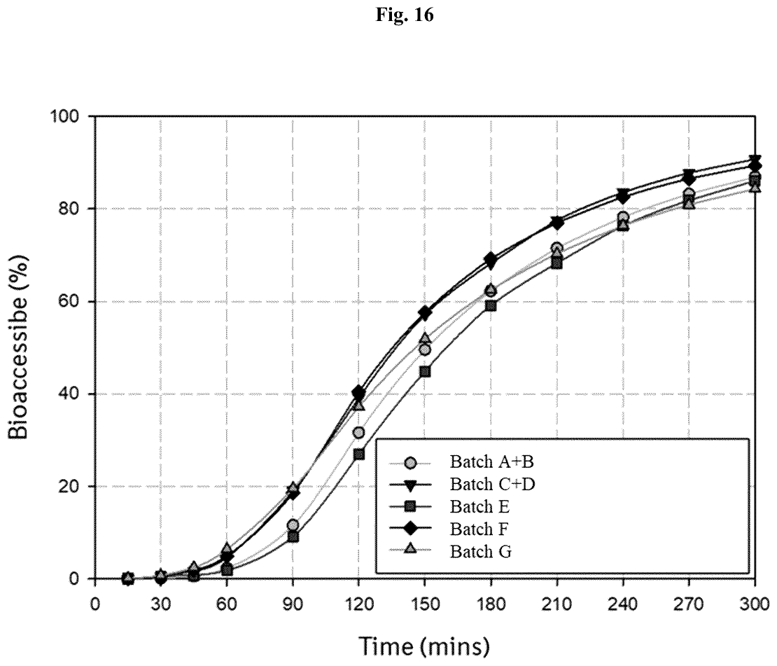

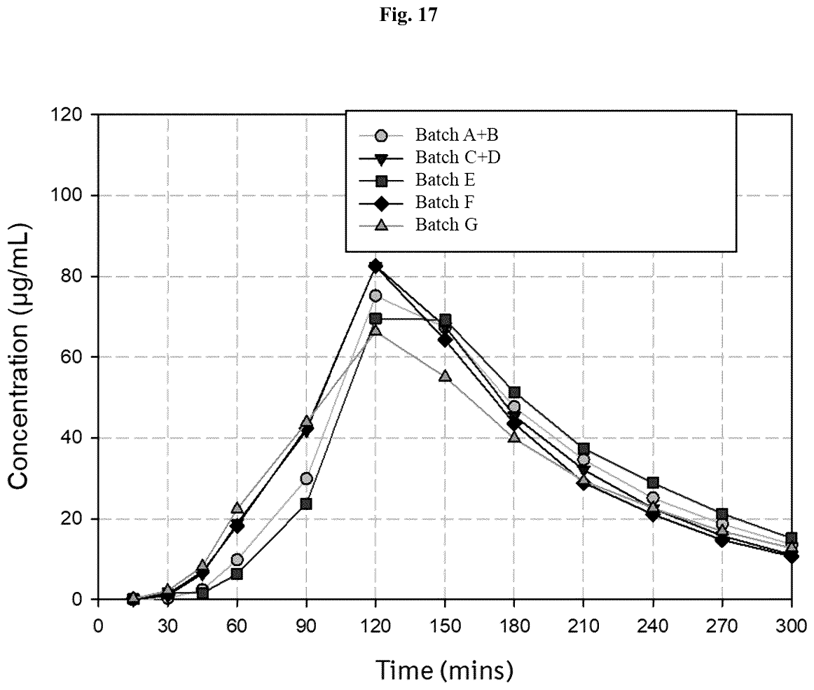

The obtained bioaccessibility and concentration profiles are displayed in FIG. 16 and FIG. 17 respectively and suggest that PSD and amorphous content did not have a significant effect. Therefore, the crystalline form of Compound A of the present invention and amorphous Compound A, have comparable bioaccessibility profiles.

### Comparative Example 2: Dissolution Study in Phosphate Buffer pH 6.8

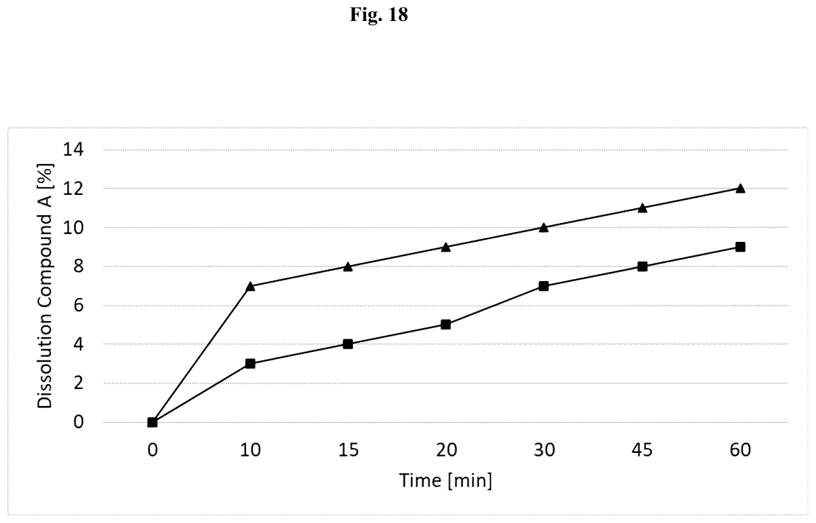

The dissolutions in phosphate buffer solution pH 6.8 were tested for formulation F6 manufactured without surfactant and formulation F5 containing 1 weight % SLS. As can be seen from FIG. 18, formulation F5 showed approximately a two-fold increase in dissolution during the first 20 minutes and after 60 minutes approx. 12 weight % of Compound A were dissolved from formulation F5 compared to only 9 weight % from formulation F6. The resulting concentration of −0.0003% SLS (completely dissolved) in the dissolution medium is far less than the critical micelle concentration. Thus, the increase in dissolution observed for formulation F5 compared to formulation F6 is mainly the result of an increased wettability of Compound A which can be attributed to the SLS in the formulation.

### Comparative Example 3: Dissolution Study in Biorelevant Media

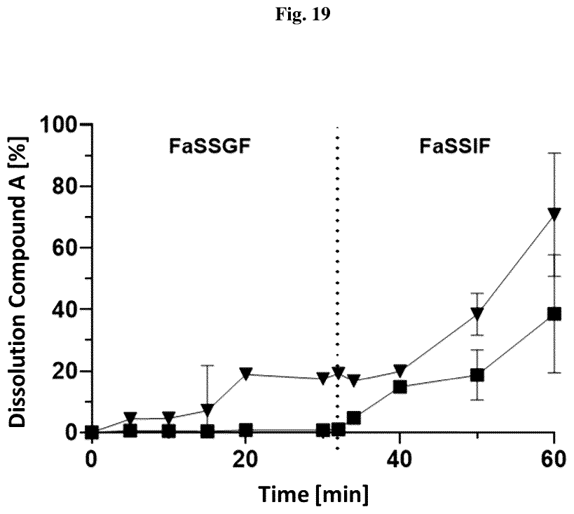

Tablet cores pressed from the final blends of formulation F6 (no surfactant present) and formulation F5 (containing SLS) were subjected to dissolution testing applying a two-step dissolution model using fasted state simulated gastric fluid (FaSSGF/pH 1.6) in a first step from 0 to 30 minutes, followed by fasted state simulated intestinal fluid (FaSSIF/pH 6.5) in a second step from 30 to 60 minutes. As can be seen from FIG. 19, approximately 20 weight % of Compound A were dissolved after 30 minutes in FaSSGF medium from the formulation F5 containing SLS compared to almost no release from the formulation F6 lacking a surfactant. Change of the dissolution medium from FaSSGF to FaSSIF also resulted into a clear difference between the two formulations. While approximately 70 weight % of Compound A dissolved from the formulation F6, only approximately 35 weight % Compound A dissolved from the formulation F5. These results further support the conclusion that an inclusion of a low amount of surfactant in the formulation increases the wettability of Compound A.

### Reference Example 1: Preparation and Characterization of Compound a DIPE Solvate

Reference Example 1.1: Preparation of Compound A DIPE solvate Compound A (e.g. the crystalline form as obtained from any one of Examples 1 to 4 hereinabove) was slurried in diisopropylether in a closed vial at room temperature for 13 days before the solid material was separated by filtration to obtain Compound A DIPE solvate.

### Reference Example 1.2: XRPD of Compound a DIPE Solvate

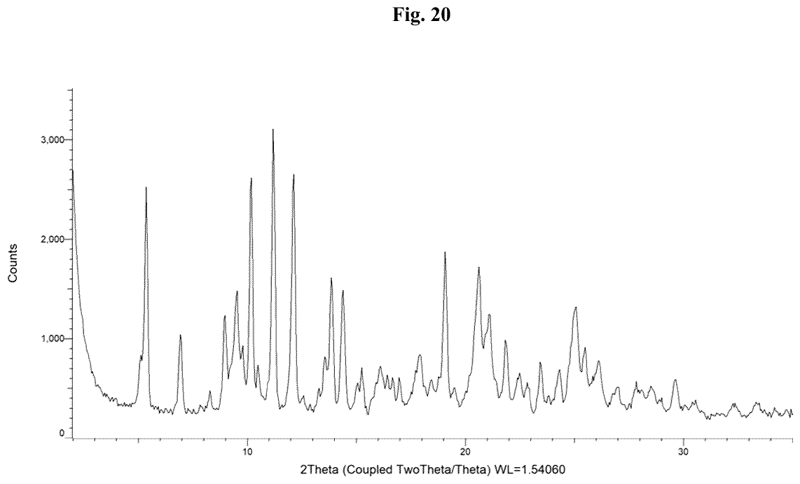

The DIPE solvate was characterized by XRPD according to the method disclosed in Example hereinabove. XRPD analysis indicated that the material is crystalline. The diffractogram of Compound A DIPE solvate is displayed in FIG. 20. The corresponding list of reflections (peaks) is provided in the following Table 16.

TABLE 16
Reflection (peak) positions of Compound A DIPE solvate
obtained from Reference Example 1.1
Reflection (peak) position
[°2-theta]

5.4
6.9
9.0
9.5
10.2
11.2
12.1
13.9
14.4
15.3
16.1
17.9
19.1
20.6
21.1
21.8
23.4
25.1
25.5
26.1

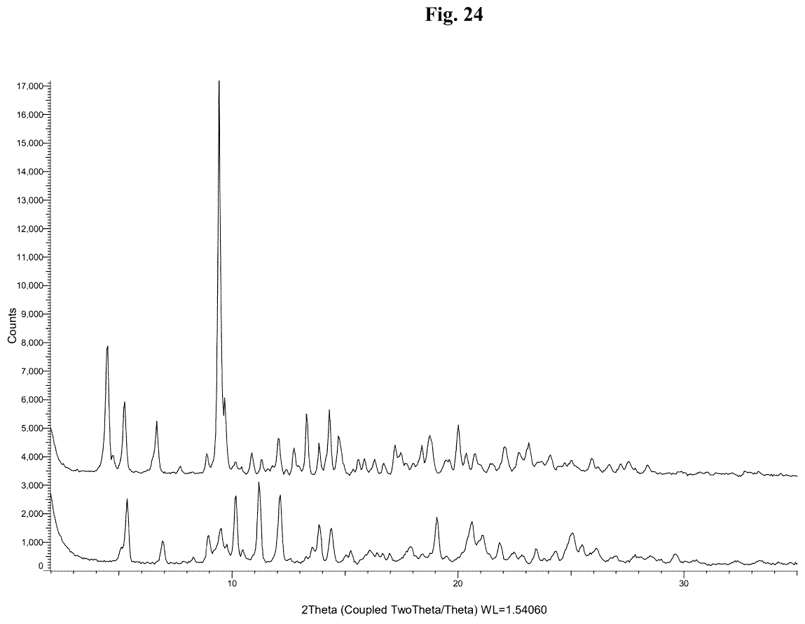

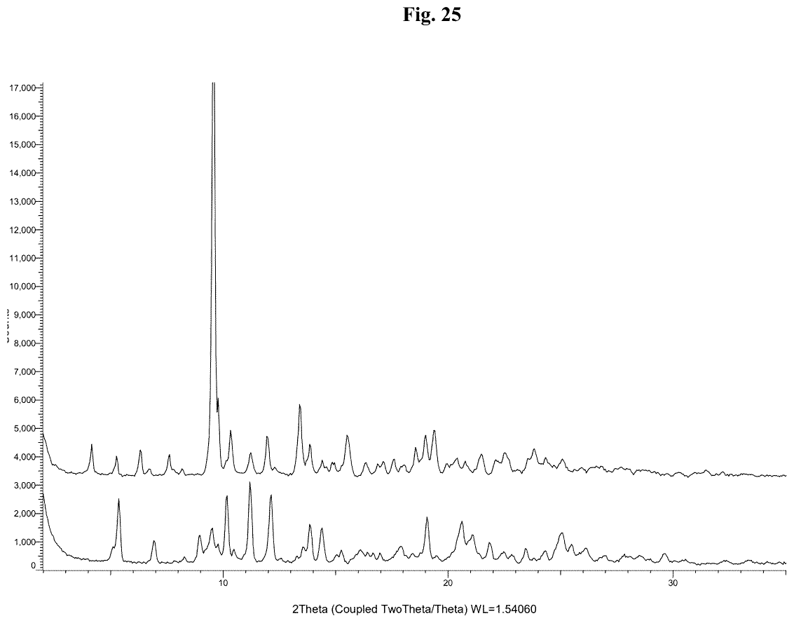

FIG. 24 and FIG. 25 illustrate a comparison of the XRPDs of Compound A DIPE solvate and the crystalline form of Compound A of the present invention prepared according to the procedures described in Example 1 and 3. As can be seen the XRPD of the DIPE solvate is significantly different.

### Reference Example 1.3: TGA of Compound a DIPE Solvate

TGA analysis was performed using a Mettler-Toledo TGA/DSC3+ analyzer. Temperature and enthalpy adjustments were performed using indium, phenyl salicylate, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen. Data was collected from 25° C. to 350° C. at 10° C./min.

The TGA curve shows a mass loss of 14.5 wt % that calculates to 1 mole of DIPE indicating the presence of a monosolvate.

### Reference Example 1.4: Stability of Compound a DIPE Solvate at 125° C. Under Vacuum

Compound A DIPE solvate was open stored in a vacuum drying oven at 125° C. for 1 day. The stressed sample was investigated by XRPD which was collected with a PANalytical X′Pert PRO MPD using an incident beam of Cu radiation produced using an Optix long, fine-focus source. An elliptically graded multilayer mirror was used to focus Cu Ka X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X′Celerator) located 240 mm from the specimen and Data Collector software v. 5.5. The following data acquisition parameters have been applied:

| | |
|---|---|
| X-ray tube | Cu (1.54056 Å) |
| Voltage | 45 kV |
| Amperage | 40 mA |
| Scan range | 1.00 −39.99 °2-theta |
| Step size | 0.017 °2-theta |
| Collection time | 722 s |
| Scan speed | 3.2°/min |
| Slit | DS: Fixed slit 1/2° |
| SS | null |
| Revolution time | 1.0 s |
| Mode | Transmission |

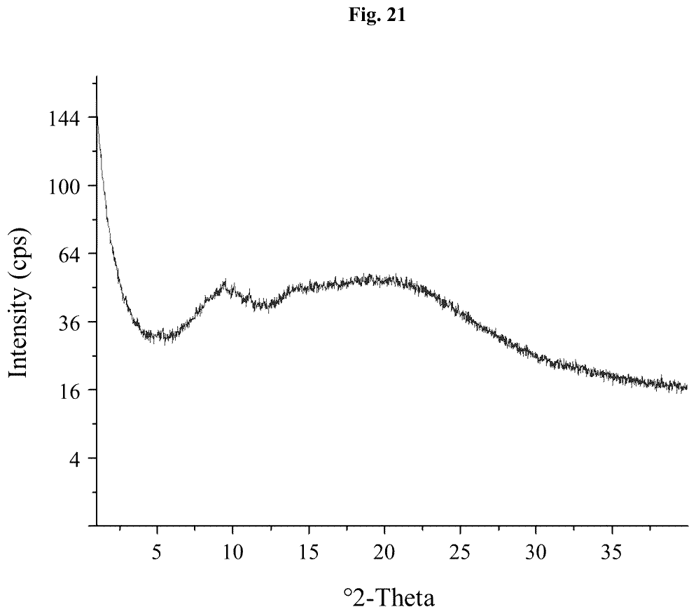

Vacuum drying of the DIPE solvate at 125° C. for 1 day resulted in mainly X-ray amorphous material (see FIG. 21), indicating the sensitivity of this crystalline form against temperature stress.

1) (2′S,3′S,3a′S,10a′S)-6-chloro-3′-(3-chloro-2-fluorophenyl)-1′-(cyclopropylmethyl)-6′-methyl-2-oxo-1,2,3′,3a′,10′,10a′-hexahydro-1′H-spiro[indole-3,2′-pyrrolo[2′,3′:4,5]pyrrolo[1,2-b]indazole]-7′-carboxylic acid of Formula (I) (=Compound A) 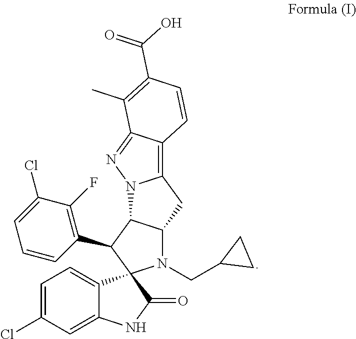 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]
in crystalline form, characterized by having an X-ray powder diffractogram comprising a reflection at 2-theta angles in the range of from 4.0° to 4.6°, when measured at a temperature in the range of from 20 to 30° C. with Cu-Kalpha radiation having a wavelength of 0.15406 nm.

2) The crystalline form according to claim 1, characterized by having an X-ray powder diffractogram comprising a reflection at 2-theta angles in the range of from 9.2° to 9.6°, when measured at a temperature in the range of from 20 to 30° C. with Cu-Kalpha radiation having a wavelength of 0.15406 nm.

3) The crystalline form according to claim 2, characterized by having an X-ray powder diffractogram comprising a reflection at 2-theta angles in the range of from 6.5 to 6.8°, when measured at a temperature in the range of from 20 to 30° C. with Cu-Kalpha radiation having a wavelength of 0.15406 nm.

4) The crystalline form of claim 1, characterized by having an X-ray powder diffractogram comprising reflections at 2-theta angles of (4.4±0.2°), (6.6±0.2°) and (9.3±0.2°), when measured at a temperature in the range of form 20 to 30° C. with Cu-Kalpha radiation having a wavelength of 0.15406 nm.

5) The crystalline form of claim 4 characterized by having an X-ray powder diffractogram comprising additional reflections at 2-theta angles of (8.7±0.2°) and (11.9±0.2°), when measured at a temperature in the range of from 20 to 30° C. with Cu-Kalpha radiation having a wavelength of 0.15406 nm.

6) The crystalline form according to claim 1 characterized by having:
(i) a Raman spectrum comprising a peak at (1680±2) cm⁻¹, when measured at a temperature in the range of from 20 to 30° C. and a wavelength of 830 nm; and/or
(ii) a ¹³C-ssNMR-spectrum comprising peaks at (18.6±0.3) ppm and/or (176.4±0.3) ppm; and/or
(iii) a ¹⁹F-ssNMR spectrum comprising peaks at (−117.5±0.5) ppm and/or (−120.3±0.5) ppm.

7) The crystalline form of claim 1, characterized by having an X-ray powder diffractogram comprising reflections at 2-theta angles of (4.2±0.2°), (6.3±0.2°) and (9.6±0.2°), when measured with Cu-Kalpha radiation having a wavelength of 0.15406 nm.

8) The crystalline form of claim 7, characterized by having an X-ray powder diffractogram comprising additional reflections at 2-theta angles of (10.3±0.2°) and (13.4±0.2°), when measured at a temperature in the range of from 20 to 30° C. with Cu-Kalpha radiation having a wavelength of 0.15406 nm.

9) The crystalline form according to claim 1, characterized by having:
(i) a Raman spectrum comprising a peak at (1732±2) cm⁻¹, when measured at a temperature in the range of from 20 to 30° C. and a wavelength of 830 nm.
(ii) a ¹³C-ssNMR spectrum comprising peaks at (16.4±0.3) ppm and/or (112.4±0.3 ppm); and/or
(iii) a ¹⁹F-ssNMR spectrum comprising peaks at (−116.2±0.5) ppm and/or (−118.6±0.5) ppm.

10) A composition comprising the crystalline form as defined in claim 1 and at most 50% (w/w) of any solid-state form of Compound A other than the crystalline form as defined in claim 1, based on the weight of the composition, wherein the other solid-state form is amorphous Compound A.

11) The composition of claim 10 comprising 5 to 30% (w/w) of amorphous Compound A.

12) The composition of claim 10 characterized by having an X-ray powder diffractogram comprising a reflection at 2-theta angles in the range of from 4.0° to 4.6°, when measured at a temperature in the range of from 20 to 30° C. with Cu-Kalpha radiation having a wavelength of 0.15406 nm.

13) The composition of claim 12, characterized by having an X-ray powder diffractogram comprising a reflection at 2-theta angles in the range of from 9.2° to 9.6°, when measured at a temperature in the range of from 20 to 30° C. with Cu-Kalpha radiation having a wavelength of 0.15406 nm.

14) The composition of claim 13, characterized by having an X-ray powder diffractogram comprising a reflection at 2-theta angles in the range of from 6.5 to 6.8°, when measured at a temperature in the range of from 20 to 30° C. with Cu-Kalpha radiation having a wavelength of 0.15406 nm.

15) A process for the preparation of the crystalline form as defined in claim 1, comprising:
(i) providing a solution of Compound A in a solvent mixture comprising water and at least one water-miscible organic solvent;
(ii) optionally, seeding the solution provided in (i) with the crystalline form as defined in claim 1;
(iii) crystallizing Compound A from the solution provided in (i), or optionally from the mixture obtained in (ii);
(iv) separating at least a part of the crystals or composition obtained in (iii) from the mother liquor;
(v) optionally, washing the crystals or composition obtained in (iv); and
(vi) drying the crystals or composition obtained in any one of steps (iv) or optionally (v).

16) (2′S,3′S,3a′S,10a′S)-6-chloro-3′-(3-chloro-2-fluorophenyl)-1′-(cyclopropylmethyl)-6′-methyl-2-oxo-1,2,3′,3a′,10′,10a′-hexahydro-1′H-spiro [indole-3,2′-pyrrolo[2′,3′:4,5]pyrrolo[1,2-b]indazole]-7′-carboxylic acid of Formula (I) (=Compound A) 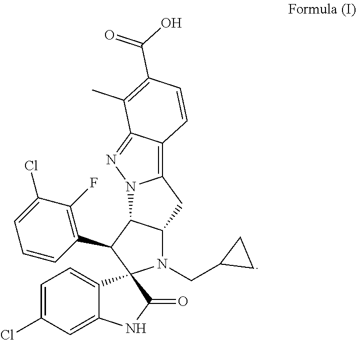 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]
in crystalline form or a composition comprising Compound A in crystalline form prepared, obtainable, or obtained by a process comprising:
(i) providing a solution of Compound A in a solvent mixture comprising water and at least one water-miscible organic solvent;
(ii) optionally, seeding the solution provided in (i) with the crystalline form as defined in claim 1;
(iii) crystallizing Compound A from the solution provided in (i), or optionally from the mixture obtained in (ii);
(iv) separating at least a part of the crystals or composition obtained in (iii) from the mother liquor;
(v) optionally, washing the crystals or composition obtained in (iv); and
(vi) drying the crystals or composition obtained in any one of steps (iv) or optionally (v).

17) Use of the crystalline form according to claim 1 for the preparation of a pharmaceutical composition.

18) A pharmaceutical composition comprising the crystalline form according to claim 1 and at least one pharmaceutically acceptable excipient.

19) The pharmaceutical composition according to claim 18, wherein the pharmaceutically acceptable excipient is selected from the group consisting of fillers (diluents), binders, surfactants, disintegrants, lubricants, and any combinations thereof.

20) The pharmaceutical composition of claim 19 comprising 0.1 to 5% (w/w) of a surfactant.

21) The pharmaceutical composition of claim 20, wherein the surfactant is an anionic surfactant.

22) The pharmaceutical composition of claim 21, wherein the surfactant is sodium lauryl sulfate.

23) The pharmaceutical composition according to claim 18 comprising a predetermined and/or effective amount of said crystalline form.

24) The pharmaceutical composition of claim 23, wherein the predetermined and/or effective amount is selected from the group consisting of 10 mg, 30 mg and 45 mg, calculated as free Compound A.

25) The pharmaceutical composition according to claim 18, characterized by having an X-ray powder diffractogram comprising a reflection at 2-theta angles in the range of from 4.0° to 4.6°, when measured at a temperature in the range of from 20 to 30° C. with Cu-Kalpha radiation having a wavelength of 0.15406 nm.

26) The pharmaceutical composition of claim 25, characterized by having an X-ray powder diffractogram comprising a reflection at 2-theta angles in the range of from 9.2° to 9.6°, when measured at a temperature in the range of from 20 to 30° C. with Cu-Kalpha radiation having a wavelength of 0.15406 nm.

27) The pharmaceutical composition of claim 26, characterized by having an X-ray powder diffractogram comprising a reflection at 2-theta angles in the range of from 6.5 to 6.8°, when measured at a temperature in the range of from 20 to 30° C. with Cu-Kalpha radiation having a wavelength of 0.15406 nm.

28) The pharmaceutical composition according to claim 18, which is an oral solid dosage form.

29) A process for the preparation of the pharmaceutical composition as defined in claim 18 comprising the steps of:
(i) mixing said crystalline form with at least one pharmaceutically acceptable excipient;
(ii) subjecting the mixture obtained in (i) to a wet granulation thereby contacting the mixture with a granulation liquid, wherein the granulation liquid comprises at least one surfactant dissolved in the granulation liquid;
(iii) drying the granules obtained in (ii);
(iv) optionally, mixing the granules obtained in (iii) with one or more additional pharmaceutically acceptable excipient(s);
(v) optionally, tableting the granules obtained in (iii) or optionally the mixture obtained in (iv);
(vi) optionally, film-coating the tablets obtained in (v).

30) A pharmaceutical composition prepared, obtainable, or obtained by a process comprising:
(i) mixing the crystalline form according to claim 1 with at least one pharmaceutically acceptable excipient;
(ii) subjecting the mixture obtained in (i) to a wet granulation thereby contacting the mixture with a granulation liquid, wherein the granulation liquid comprises at least one surfactant dissolved in the granulation liquid;
(iii) drying the granules obtained in (ii);
(iv) optionally, mixing the granules obtained in (iii) with one or more additional pharmaceutically acceptable excipient(s);
(v) optionally, tableting the granules obtained in (iii) or optionally the mixture obtained in (iv);
(vi) optionally, film-coating the tablets obtained in (v).

31) The crystalline form according to claim 1 for use as a medicament.

32) The crystalline form according to claim 1 for use in the treatment of cancer.

33) The crystalline form according to claim 32, wherein the cancer is a p53 wildtype form of cancer.

34) The crystalline form according to claim 32, wherein the cancer is liposarcoma including well differentiated liposarcoma (WDLPS) and dedifferentiated liposarcoma (DDLPS).

35) The crystalline form according to claim 31, wherein a predetermined and/or effective amount of the crystalline form, the composition, or the pharmaceutical composition is to be administered to a subject intermittently in at least three consecutive doses and the period between each two consecutive doses is at least three weeks and no longer than sixty days.

36) The crystalline form according to claim 31, wherein a predetermined and/or effective amount of the crystalline form, the composition, or the pharmaceutical composition is to be administered to a subject intermittently and the period between consecutive administrations is three weeks.

37) A pharmaceutical composition comprising (2′S,3′S,3a′S,10a′S)-6-chloro-3′-(3-chloro-2-fluorophenyl)-1′-(cyclopropylmethyl)-6′-methyl-2-oxo-1,2,3′,3a′,10′,10a′-hexahydro-1′H-spiro[indole-3,2′-pyrrolo[2′,3′:4,5]pyrrolo[1,2-b]indazole]-7′-carboxylic acid of Formula (I) (=Compound A) 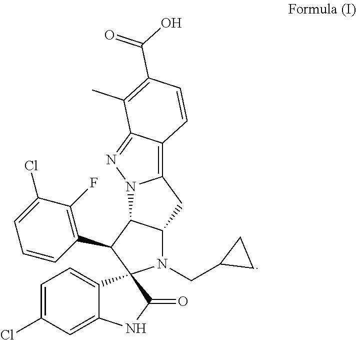 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]
0.1 to 5% (w/w) surfactant, and least one further pharmaceutically acceptable excipient.

38) The pharmaceutical composition according to claim 37, wherein the surfactant is an anionic surfactant.

39) The pharmaceutical composition of claim 38, wherein the surfactant is sodium dodecyl sulfate.

40) The pharmaceutical composition of claim 38, wherein the pharmaceutically acceptable excipient is selected from the group consisting of fillers (diluents), binders, disintegrants, lubricants, and any combinations thereof.
